(19) United States
(12) Patent Application Publication
(10) Pub. No.: US 2023/0201174 A1
(43) Pub. Date: Jun. 29, 2023

(54) COMBINATION OF FINERENONE AND A SGLT2 INHIBITOR FOR THE TREATMENT AND/OR PREVENTION OF CARDIOVASCULAR AND/OR RENAL DISEASES

(71) Applicant: BAYER AKTIENGESELLSCHAFT, Leverkusen (DE)

(72) Inventors: Peter KOLKHOF, Wuppertal (DE); Peter SANDNER, Wuppertal (DE); Lars BÄRFACKER, Düsseldorf (DE)

(21) Appl. No.: 17/996,816

(22) PCT Filed: Apr. 20, 2021
(86) PCT No.: PCT/EP2021/060176

§ 371 Date: Oct. 21, 2022

(30) Foreign Application Priority Data
Apr. 22, 2020 (EP) 20170936.7
May 6, 2020 (EP) 20173299.7
May 25, 2020 (EP) 20176338.0
Aug. 21, 2020 (EP) 20192072.5
Feb. 5, 2021 (EP) 21155489.4

(51) Int. Cl. A61K 31/4375; A61K 31/7034; A61P 3/10; A61P 9/00; A61P 13/12

(52) U.S. Cl. CPC A61K 31/4375; A61K 31/7034; A61P 3/10; A61P 9/00; A61P 13/12

(57) ABSTRACT

The present invention relates to pharmaceutical compositions and combinations comprising finerenone or a hydrate, solvate or pharmaceutically acceptable salt thereof or a polymorph thereof and a SGLT2 inhibitor, or a hydrate, solvate or pharmaceutically acceptable salt thereof or a polymorph thereof. The combination can be used for the treatment and/or prevention of cardiovascular and/or renal diseases in humans and other mammals.

The invention refers to the combination of finerenone or a hydrate, solvate, pharmaceutically acceptable salt thereof, or a polymorph thereof, and a SGLT2 inhibitor a hydrate, solvate, pharmaceutically acceptable salt thereof, or a polymorph thereof, for the treatment and/or prevention of cardiovascular and/or renal diseases. In particular, these diseases can be characterized by chronic sodium retention.

The liquid content of the human body is subject to various physiological control mechanisms, the purpose of which is to keep it constant (volume homeostasis). In the process, both the volume filling of the vascular system and also the osmolarity of the plasma are continuously recorded by appropriate sensors (baroreceptors and osmoreceptors). The information which these sensors supply to the relevant centers in the brain regulates drinking behavior and controls fluid excretion via the kidneys by means of humoral and neural signals.

The steroid hormone aldosterone plays a key part in maintaining fluid and electrolyte homeostasis by promoting, in the epithelium of the distal nephron, sodium retention and potassium secretion, thus, contributing to keep the extracellular volume constant and, thus, to regulate blood pressure. Besides this, aldosterone displays direct effects on the structure and function of the cardiac and vascular system, but the underlying mechanisms thereof are not yet fully explained (R. E. Booth, J. P. Johnson, J. D. Stockand, Adv. Physiol. Educ. 26 (1), 8-20 (2002)).

Finerenone, which is (S)-4-(4-cyano-2-methoxyphenyl)-5-ethoxy-2,8-dimethyl-1,4-dihydro-1,6-naphthyridin-3-carboxamide, a compound of formula (I)

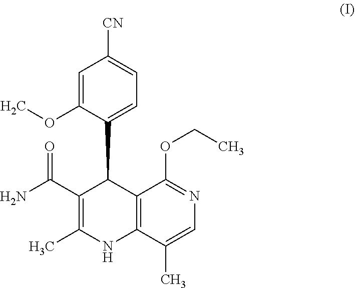 [SMILES: CCOc1ncc(C)c2c1[C@H](c1ccc(C#N)cc1OC)C(C(N)=O)=C(C)N2]

is a mineralocorticoid receptor antagonist (MR antagonist). The synthesis, pharmacological properties and pharmaceutical formulations/dosage forms are described in detail in WO 2008/104306 A1. WO 2008/104306 A1 states that the MR IC50 of 4-(4-cyano-2-methoxyphenyl)-5-ethoxy-2,8-dimethyl-1,4-dihydro-1,6-naphthyridine-3-carboxamide (racemate, Example 4) is 23 nM and the IC50 of ent-4-(4-cyano-2-methoxyphenyl)-5-ethoxy-2,8-dimethyl-1,4-dihydro-1,6-naphthyridine-3-carboxamide ((−)-enantiomer, Example 5; “finerenone”) is 16 nM (see WO2008/104306 A1, section B.1 “Assessment of the pharmacological activity”). A further process for the manufacturing of finerenone is described in WO 2016/016287 A1. MR antagonists (such as the steroidal compounds spironolactone, canrenone/canrenoate and eplerenone, and also more recent non-steroidal MR antagonists such as MT-3995, CS-3150, AZD9977, KBP-5074, LY2623091, PF-03882845 and finerenone) counteract aldosterone-mediated sodium retention in the kidneys (natriuretic effect). Thus, MR antagonists lead to increased sodium excretion, which is a proven therapeutic concept for hypertensive patients and/or patients suffering from heart failure and/or kidney failure. However, MR antagonists can unfold their natriuretic action only in kidney segments in which aldosterone also exerts its physiological action via the MR. These are in particular the late distal tubulus and collecting duct sections involved in sodium re-resorption only to a limited extent, whereas most of sodium secretion and re-resorption takes place in proximal tubulus sections. In absolute terms, aldosterone only influences about 2% of the total sodium reabsorption via MR in the distal tubule and in the collecting tube, thereby limiting the natriuretic potency of MR antagonists. Although use of MR antagonists is already part of the pharmacological treatment according to guidelines for chronic heart failure patients, the natriuretic potency of MR antagonists is limited.

The sodium-glucose co-transporter-2 (SGLT2) is expressed in the proximal renal tubule and reabsorbs ca. 97% of filtered glucose. Since SGLT2 inhibitors block the reabsorption of both glucose and sodium, they act glycosuric and natriuretic, and as a consequence, act antihyperglycemic, diuretic, lower blood pressure, reduce body weight, and increase insulin sensitivity. This translates in outcome benefits among diabetic patients with chronic heart failure (CHF) and chronic kidney disease (CKD). Interestingly, it has been demonstrated most recently that SGLT2 inhibition exert cardiovascular and renal benefits that are not mediated through changes in blood glucose, i.e. morbidity and mortality reduction in both, diabetic as well as non-diabetic patients. The precise beneficial mode of action of SGLT2 inhibition especially in non-diabetic CHF and/or CKD is not fully understood but one important component of these benefits is the natriuretic activity since pathological hyper-reabsorption of sodium in the early proximal tubule is mediated primarily by increased activity of SGLT2 in the diabetic kidney [Vallon V. The mechanisms and therapeutic potential of SGLT2 inhibitors in diabetes mellitus. Annu Rev Med. 2015; 66:255-70]. This augmented reabsorption reduces the delivery of sodium to the macula densa, which, through tubule-glomerular feedback, causes afferent arteriolar vasodilation and glomerular hyperfiltration. Attenuation of this proximal tubular hyper-reabsorption of sodium by SGLT2 causes afferent vasoconstriction, thereby lowering glomerular hyperfiltration and renal damage, and improves renal function in the long-term. However, the natriuretic effect of SGLT2 inhibitors has been reported to be only transient, e.g. natriuresis was dose-dependently induced by dapagliflozin during the first 24 h in healthy volunteers but returned to baseline after 13 days of intervention. Another previous study showed that urinary Na+ excretion tended to increase on day 1 after administration of canagliflozin but returned to baseline from day 2 to 5.

Therefore, although SGLT2 inhibitors can potently induce glycosuria and thus osmotic diuresis, their natriuretic efficacy seems to be limited.

Known SGLT2 inhibitor are canagliflozin, dapagliflozin, empagliflozin, ertugliflozin, ipragliflozin, remogliflozin, sergliflozin, sotagliflozin and tofogliflozin.

Canagliflozin, which is (2S,3R,4R,5S,6R)-2-(3-{[5-(4-fluorophenyl)thiophen-2-yl]methyl}-4-methylphenyl)-6-(hydroxymethyl)oxane-3,4,5-triol (CAS-number: 842133-18-0) and is depicted in formula (II) or (IIa) (hemihydrate) below

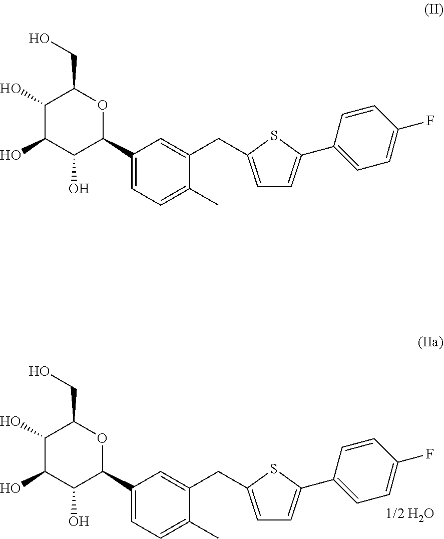 [SMILES: Cc1ccc([C@@H]2O[C@H](CO)[C@@H](O)[C@H](O)[C@H]2O)cc1Cc1ccc(-c2ccc(F)cc2)s1 ; Cc1ccc([C@@H]2O[C@H](CO)[C@@H](O)[C@H](O)[C@H]2O)cc1Cc1ccc(-c2ccc(F)cc2)s1]

is also known as Invokana® (market product by e.g. Janssen Pharmaceuticals, comprises canagliflozin hydrate), is a sodium-glucose cotransporter 2 (SGLT2) inhibitor used in the management of type 2 diabetes mellitus along with lifestyle changes including diet and exercise. It is described in U.S. Pat. Nos. 7,943,582, 7,943,788, 8,222,219 and 8,513,202. It was initially approved by the FDA in 2013 for the management of diabetes and later approved in 2018 for a second indication of reducing the risk of cardiovascular events in patients diagnosed with type 2 diabetes mellitus. Canagliflozin is the first oral antidiabetic drug approved for the prevention of cardiovascular events in patients with type 2 diabetes. Cardiovascular disease is the most common cause of death in these patients.

Dapagliflozin, which is (2S,3R,4R,5S,6R)-2-[4-chloro-3-[(4-ethoxyphenyl)methyl]phenyl]-6-(hydroxymethyl)oxane-3,4,5-triol and is depicted in formula (III) below

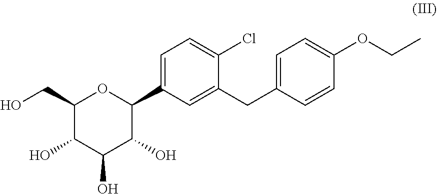 [SMILES: CCOc1ccc(Cc2cc([C@@H]3O[C@H](CO)[C@@H](O)[C@H](O)[C@H]3O)ccc2Cl)cc1]

is sold under the brand name Farxiga® among others, is a medication used to treat type 2 diabetes and, with certain restrictions, type 1 diabetes. It is of the gliflozin class. It was developed by Bristol-Myers Squibb in partnership with AstraZeneca. Dapagliflozin is described in U.S. Pat. Nos. 6,414,126, 6,515,117, 6,936,590, 7,456,254, 7,851,502, 7,919,598, 8,221,786, 8,329,648, 8,361,972, 8,431,685, 8,461,105, 8,501,698, 8,685,934, 8,716,251, 8,721,615, 8,906,851, 9,198,925 and 9,238,076.

Empagliflozin, which is (2S,3R,4R,5S,6R)-2-[4-chloro-3-[[4-[(3S)-oxolan-3-yl]oxyphenyl]methyl]phenyl]-6-(hydroxymethyl)oxane-3,4,5-triol and depicted ion formula (IV) below

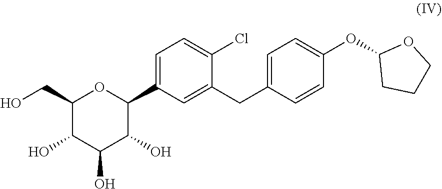 [SMILES: OC[C@H]1O[C@@H](c2ccc(Cl)c(Cc3ccc(O[C@H]4CCCO4)cc3)c2)[C@H](O)[C@@H](O)[C@@H]1O]

is sold under the brand name Jardiance among others, is a medication used together with diet and exercise to treat type 2 diabetes. It is described in U.S. Pat. Nos. 7,579,449, 7,713,938, 8,551,957, 9,949,997, 9,949,998 and U.S. Ser. No. 10/258,637.

Ertugliflozin, which is (1S,2S,3S,4R,5S)-5-[4-chloro-3-[(4-ethoxyphenyl)methyl]phenyl]-1-(hydroxymethyl)-6,8-dioxabicyclo[3.2.1]octane-2,3,4-triol and is depicted in formula (V) below

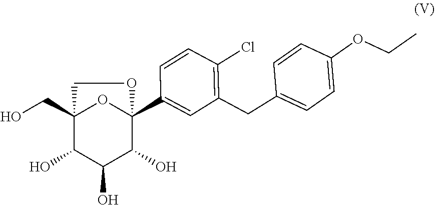 [SMILES: CCOc1ccc(Cc2cc([C@]34OC[C@](CO)(O3)[C@@H](O)[C@H](O)[C@H]4O)ccc2Cl)cc1]

is marketed under the trade name Steglatro® and is a drug for the treatment of type 2 diabetes. It is described in U.S. Pat. No. 8,080,580. In the United States, it was approved by the Food and Drug Administration for use as a monotherapy and as a fixed dose combination with either sitagliptin or with metformin.

Ipragliflozin, which is and is depicted in formula (VI) below

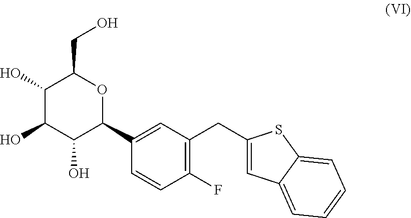 [SMILES: OC[C@H]1O[C@@H](c2ccc(F)c(Cc3cc4ccccc4s3)c2)[C@H](O)[C@@H](O)[C@@H]1O]

is a pharmaceutical drug for treatment of type 2 diabetes. Ipragliflozin, jointly developed by Astellas Pharma and Kotobuki Pharmaceutical, was approved in Japan on Jan. 17, 2014, and in Russia on May 22, 2019. The trade name is Suglat®. The compound and methods of its synthesis are described in WO 2004/080990 A1, WO 2005/012326 A1 and WO 2007/114475 A1.

Remogliflozin, which is depicted in formula (VII) below, and remogliflozin etabonate, which is depicted in formula (VIIa) below,

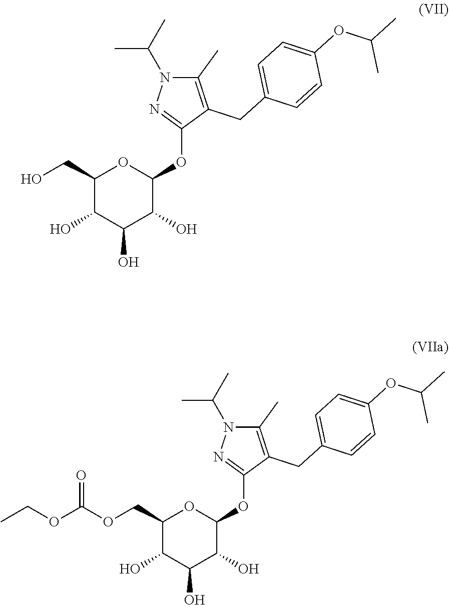

is a drug of the gliflozin class for the treatment of non-alcoholic steatohepatitis (“NASH”) and type 2 diabetes. Remogliflozin was discovered by the Japanese company Kissei Pharmaceutical and is currently being developed by BHV Pharma. It is used as remogliflozin etabonate (ethyl [(2R,3S,4S,5R,6S)-3,4,5-trihydroxy-6-[5-methyl-1-propan-2-yl-4-[(4-propan-2-yloxyphenyl)methyl]pyrazol-3-yl]oxyoxan-2-yl]methyl carbonate; CAS 442201-24-3; remogliflozin etabonate). “Etabonate” refers to the ethyl carbonate group. The remaining structure, which is the active substance, is called remogliflozin.

Sergliflozin, which is depicted in formula (VIII) and is (2R,3S,4S,5R,6S)-2-(hydroxymethyl)-6-[2-[(4-methoxyphenyl)methyl]phenoxy]oxane-3,4,5-triol, and sergliflozin etabonate, which is depicted in formula (VIIIa) below and is ethyl [(2R,3S,4S,5R,6S)-3,4,5-trihydroxy-6-[2-[(4-methoxyphenyl)methyl]phenoxy]oxan-2-yl]methyl carbonate,

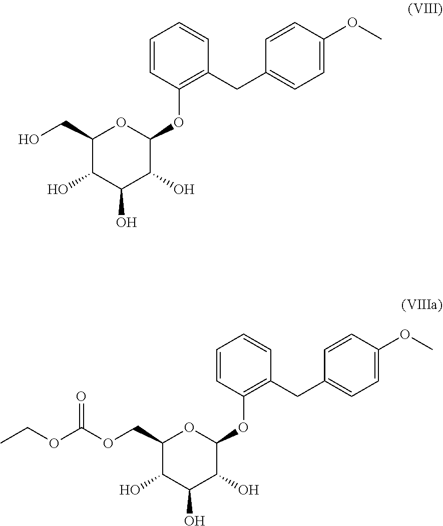

is being developed by GlaxoSmithKline in form of sergliflozin etabonate and is an investigational antidiabetic drug. “Etabonate” refers to the ethyl carbonate group. The remaining structure, which is the active substance, is called sergliflozin.

Sotagliflozin, which is (2S,3R,4R,5S,6R)-2-[4-chloro-3-[(4-ethoxyphenyl)methyl]phenyl]-6-methylsulfanyloxane-3,4,5-triol and is depicted in formula (IX) below

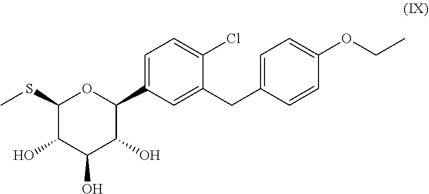 [SMILES: CCOc1ccc(Cc2cc([C@@H]3O[C@H](SC)[C@@H](O)[C@H](O)[C@H]3O)ccc2Cl)cc1]

is a drug approved in EU for certain patients with type I diabetes. It is market under the trade name Zynquista® and it was developed by Sanofi. WO 2017202351 A1 describes a crystal form of sotagliflozin, a method for preparation thereof and use thereof.

Tofogliflozin, which is and is depicted in formula (X) below,

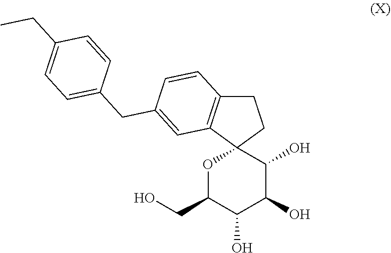 [SMILES: CCc1ccc(Cc2ccc3c(c2)[C@]2(CC3)O[C@H](CO)[C@@H](O)[C@H](O)[C@H]2O)cc1]

is a drug for the treatment of diabetes mellitus and is being developed by Chugai Pharma in collaboration with Kowa and Sanofi. It is an inhibitor of SGLT2. The United States Adopted Name tofogliflozin applies to the monohydrate, which is the form used as a drug. The International Nonproprietary Name tofogliflozin applies to the anhydrous compound and the drug form is referred to as tofogliflozin hydrate.

In summary, sodium excretion via the urine (‘natriuresis’) is a particular therapeutic target for cardiovascular diseases. For example, neurohumoral stimulation by the renin-angiotensin-aldosterone system (RAAS) or elevation of SGLT2 expression in the proximal tubule contributes to permanent sodium retention and an associated extracellular volume load in the development of chronic heart and kidney failure. The body is no longer able to perform effective natriuresis, especially when there is impaired kidney function, which is often associated with heart failure. Therefore, maintaining an effective natriuresis would be useful in treatment and/or prevention of cardiovascular and/or renal diseases.

An object of the present invention is the improvement of the treatment and/or prevention of cardiovascular and renal diseases in comparison to the already known monotherapies.

A further object of the present invention is to provide combinations of active pharmaceutical ingredients for the treatment of cardiovascular diseases, which are characterized by chronic sodium retention, in particular also cardiac and renal insufficiency, which reduce mortality and/or morbidity in patients.

Another object of the present invention is the improvement of the treatment and/or prevention of cardiovascular and renal diseases by administration of a combination comprising finerenone and a SGLT2 inhibitor.

The present invention pertains to a combination comprising finerenone with a SGLT2 inhibitor.

The invention provides for combinations of active pharmaceutical ingredients, namely the combination of finerenone with a SGLT2 inhibitor, for the treatment of cardiovascular diseases which are characterized by chronic sodium retention or kidney diseases. Cardiovascular diseases are for example congestive heart failure independent of ejection fraction (i.e. with preserved ejection fraction (HFpEF), with mid-range ejection fraction (HFmrEF) or heart failure with reduced ejection fraction (HFrEF)). Renal diseases are for example chronic kidney disease (CKD) and diabetic kidney disease, as well as other syndromes, for example cardiorenal syndrome, nephrotic syndrome, hepatorenal syndrome or end organ damage affecting the heart and kidney, which contribute to an increased mortality and/or morbidity in patients.

Surprisingly the combination of finerenone with a SGLT2 inhibitor shows a significant efficacy improvement over the sum of the monotherapies:

The combination of finerenone with a SGLT2 inhibitor leads to over-additive sodium excretion in comparison to the respective monotherapy with finerenone or a SGLT2 inhibitor alone. This over-additive effect was not predictable and goes beyond the pure addition of the effects of the monotherapies with finerenone and a SGLT2 inhibitor. The over-additive effect achieved with the combination according to the invention was also not predictable as finerenone and SGLT2 inhibitor target different receptors and/or points of the homeostasis, respectively.

This over-additive sodium excretion effected by the combination according to the invention leads to a subsequent congestion relief since sodium is the most important ion in the body controlling volume homeostasis. A further water excretion takes place through sodium excretion. The water excretion removes unwanted water retention in body tissue. Among other things, there is also a reduction in preload in the heart. The cardiovascular system is relieved. An therapeutic improvement is thus achieved by the combination according to the invention. The combination therapy according to the invention can permanently relieve the overall circulation. This natriuretic efficacy improvement is a major clinical goal in the treatment of cardiovascular and renal diseases.

The combination according to the invention could provide for the administration of lesser amounts of the administered therapeutic agents. The combination according to the invention could provide for a longer survival time among treated patients compared to standard treatments.

Finally, some preliminary data strongly suggests that both, finerenone and SGLT2 inhibitors confer renal protection in preclinical non-diabetic, hypertensive kidney disease. Combination of these two modes of action at low dosages revealed efficacious reduction in proteinuria and mortality indicating a strong potential for combined clinical use in respective cardiorenal patient populations.

The present invention refers to a combination comprising finerenone, which is the compound of the formula (I), or a hydrate, solvate or pharmaceutically acceptable salt of thereof, or a polymorph thereof, and a SGLT2 inhibitor, which is the compound of formula (II), or a hydrate, solvate or pharmaceutically acceptable salt of thereof, or a polymorph thereof.

The “active ingredients” used in the combination according to the invention are finerenone and a SGLT2 inhibitor. The term “the compound of formula (I)” or “finerenone” refers to ((S)-4-(4-cyano-2-methoxyphenyl)-5-ethoxy-2,8-dimethyl-1,4-dihydro-1,6-naphthyridin-3-carboxamide or ent-4-(4-cyano-2-methoxyphenyl)-5-ethoxy-2,8-dimethyl-1,4-dihydro-1,6-naphthyridine-3-carboxamide (WO 2008/104306 A1, Example 5, (−)enantiomer) as depicted in formula (I), or a hydrate, solvate, pharmaceutically acceptable salt thereof, or a polymorph thereof. ((S)-4-(4-cyano-2-methoxyphenyl)-5-ethoxy-2,8-dimethyl-1,4-dihydro-1,6-naphthyridin-3-carboxamide and ent-4-(4-cyano-2-methoxyphenyl)-5-ethoxy-2,8-dimethyl-1,4-dihydro-1,6-naphthyridine-3-carboxamide are synonyms. In the following, when referring herein to “finerenone”, also a hydrate, solvate, pharmaceutically acceptable salt thereof, or a polymorph thereof is meant. Thus, “finerenone” is sometimes used as synonym for “finerenone, or a hydrate, solvate, pharmaceutically acceptable salt thereof, or a polymorph thereof.” According to the invention, finerenone can be used in the combination in form of an hydrate, solvate, pharmaceutically acceptable salt thereof, or a polymorph thereof. A polymorph of finerenone is described in WO201616287A1. The term “finerenone” also comprises the anhydrate thereof.

The term “SGLT2 inhibitor” is known in the art. SGLT2 inhibitors, or gliflozins, act by inhibiting the sodium-glucose transport protein 2 (SGLT2) and thereby inhibit reabsorption of glucose in the kidney and thus lower blood sugar. Accordingly, SGLT2 inhibitors are used in the treatment of type II diabetes mellitus (T2DM).

Examples of SGLT2 inhibitors are canagliflozin, dapagliflozin, empagliflozin, ertugliflozin, ipragliflozin, remogliflozin, sergliflozin, sotagliflozin and tofogliflozin. This list is not exhaustive. The term SGLT2 inhibitor comprises an anhydrate thereof, hydrate thereof, solvate thereof, pharmaceutically acceptable salt thereof, a prodrug thereof, or a polymorph thereof. In the following, when referring herein to “SGLT2 inhibitor”, also a hydrate, solvate, pharmaceutically acceptable salt thereof, a prodrug or a polymorph thereof is meant.

In one embodiment the combination according to the invention comprises two or more SGLT2 inhibitors.

In one embodiment the SGLT2 inhibitor used in the combination according to the invention is selected from the group consisting of: canagliflozin, dapagliflozin, empagliflozin, ertugliflozin, ipragliflozin, remogliflozin, sergliflozin, sotagliflozin and tofogliflozin.

In one embodiment the SGLT2 inhibitor used in the combination according to the invention is selected from the group consisting of canagliflozin, dapagliflozin and empagliflozin.

In one embodiment the SGLT2 inhibitor is canagliflozin. The term “compound according to formula (II)” or “compound according to formula (IIa)” or “canagliflozin” refers to (2S,3R,4R,5S,6R)-2-(3-{[5-(4-fluorophenyl)thiophen-2-yl]methyl}-4-methylphenyl)-6-(hydroxymethyl)oxane-3,4,5-triol (CAS-number: 842133-18-0) or refers to an anhydrate thereof, hydrate thereof, solvate thereof, pharmaceutically acceptable salt thereof, a prodrug thereof, or a polymorph thereof, (1S)-1,5-anhydro-1-[3-[[5-(4-fluorophenyl)-2-thienyl]-methyl]-4-methylphenyl]-D-glucitol or refers to an anhydrate thereof, hydrate thereof, solvate thereof, pharmaceutically acceptable salt thereof, a prodrug thereof, or a polymorph thereof “Canagliflozin” is also known under the following terms: (1S)-1,5-anhydro-1-[3-[[5-(4-fluorophenyl)-2-thienyl]-methyl]-4-methylphenyl]-D-glucitol hemihydrate or the marketed product Invokana® (comprises canagliflozin hydrate, CAS: 928672-86-0), TA 7284, cagliflozin; canagliflozin, JNJ 28431754, canaglifozion, JNJ 24831754ZAE, JNJ 28431754AAA. The list is not exhaustive as there a several marketed product on the market comprising canagliflozin or further synonyms, which are included in the term. In the following, when referring herein to “canagliflozin”, also the aforementioned synonyms/terms are meant.

In one embodiment the SGLT2 inhibitor is dapagliflozin. The term “compound according to formula (III)” or “dapagliflozin” refers to (2S,3R,4R,5S,6R)-2-[4-chloro-3-[(4-ethoxyphenyl)methyl]phenyl]-6-(hydroxymethyl)oxane-3,4,5-triol (CAS number: 461432-26-8) or refers to an anhydrate thereof, hydrate thereof, solvate thereof, pharmaceutically acceptable salt thereof, a prodrug thereof, or a polymorph thereof “Dapagliflozin” is also known under the following terms: BMS-512148, Forxiga®, Farxiga®, BMS 512148, UNII-1ULL0QJ8UC, 1ULL0QJ8UC and (1S)-1,5-Anhydro-1-C-[4-chloro-3-[(4-ethoxyphenyl)methyl]phenyl]-D-glucitol. The list is not exhaustive as there a several marketed product on the market comprising dapagliflozin or further synonyms, which are included in the term. In the following, when referring herein to “dapagliflozin”, all the aforementioned synonyms/terms are meant.

In one embodiment the SGLT2 inhibitor is empagliflozin. The term “compound according to formula (IV)” or “empagliflozin” refers to (2S,3R,4R,5S,6R)-2-[4-chloro-3-[[4-[(3S)-oxolan-3-yl]oxyphenyl]methyl]phenyl]-6-(hydroxymethyl)oxane-3,4,5-triol (CAS number: 864070-44-0) refers to an anhydrate thereof, hydrate thereof, solvate thereof, pharmaceutically acceptable salt thereof, a prodrug thereof, or a polymorph thereof. “Empagliflozin” is also known under the following terms: 1-chloro-4-(glucopyranos-1-yl)-2-(4-(tetrahydrofuran-3-yloxy)benzyl)benzene, Jardiance®, BI 10773, BI-10773 and BI10773. The list is not exhaustive as there a several marketed product on the market comprising empagliflozin or further synonyms, which are included in the term. In the following, when referring herein to “empagliflozin”, all the aforementioned synonyms/terms are meant.

In one embodiment the SGLT2 inhibitor is The term “compound according to formula (V)” or “ertugliflozin” refers to (1S,2S,3S,4R,5S)-5-[4-chloro-3-[(4-ethoxyphenyl)methyl]phenyl]-1-(hydroxymethyl)-6,8-dioxabicyclo[3.2.1]octane-2,3,4-triol (CAS number: 1210344-57-2) refers to an anhydrate thereof, hydrate thereof, solvate thereof, pharmaceutically acceptable salt thereof, a prodrug thereof, or a polymorph thereof. “Ertugliflozin” is also known under the following terms: 5-(4-chloro-3-(4-ethoxybenzyl)phenyl)-1-hydroxymethyl-6,8-dioxabicyclo(3.2.1)octane-2,3,4-triol, PF 04971729, PF-04971729, PF04971729 and 1,6-Anhydro-1-C-[4-chloro-3-[(4-ethoxyphenyl)methyl]phenyl]-5-C-(hydroxymethyl)-beta-L-idopyranose. The list is not exhaustive as there a several marketed product on the market comprising ertugliflozin or further synonyms, which are included in the term. In the following, when referring herein to “ertugliflozin”, all the aforementioned synonyms/terms are meant.

In one embodiment the SGLT2 inhibitor is ipragliflozin. The term “compound according to formula (VII)” or “ipragliflozin” refers to (2S,3R,4R,5S,6R)-2-[3-(1-benzothiophen-2-ylmethyl)-4-fluorophenyl]-6-(hydroxymethyl)oxane-3,4,5-triol (CAS number: 761423-87-4) refers to an anhydrate thereof, hydrate thereof, solvate thereof, pharmaceutically acceptable salt thereof, a prodrug thereof, or a polymorph thereof “Ipragliflozin” is also known under the following terms: ipragliflozin L-proline ((2S,3R,4R,5S,6R)-2-[3-(1-benzothiophen-2-ylmethyl)-4-fluorophenyl]-6-(hydroxymethyl)oxane-3,4,5-triol; (2S)-pyrrolidine-2-carboxylic acid; CAS number: 951382-34-6), (1S)-1,5-anhydro-1-(3-(1-benzothiophen-2-ylmethyl)-4-fluorophenyl)-D-glucitol, ASP-1941, ASP1941 and Suglat® (trade name). The list is not exhaustive as there a several marketed product on the market comprising “ipragliflozin” or further synonyms, which are included in the term. In the following, when referring herein to “ipragliflozin”, all the aforementioned synonyms/terms are meant.

In one embodiment the SGLT2 inhibitor is remogliflozin. The term “compound according to formula (VII)” or “compound according to formula (VIIa)” or “remogliflozin” or remogliflozin refers to refers to an anhydrate thereof, hydrate thereof, solvate thereof, pharmaceutically acceptable salt thereof, a prodrug thereof, or a polymorph thereof. “Remogliflozin” is also known under the following terms: remogliflozin etabonate, UNII-TR0QT6QSUL, GSK-189075, TR0QT6QSUL, 442201-24-3, GSK 189075, KGT-1681, GSK-189075A, or SCHEMBL721678. The list is not exhaustive as there a several marketed product on the market comprising “remogliflozin” or further synonyms, which are included in the term. In the following, when referring herein to “remogliflozin”, all the aforementioned synonyms/terms including “remogliflozin etabonate” or any anhydrate, hydrate, solvate, pharmaceutically acceptable salt thereof are meant.

In one embodiment the SGLT2 inhibitor is sergliflozin. The term “compound according to formula (VIII)” or “sergliflozin” refers to an anhydrate thereof, hydrate thereof, solvate thereof, pharmaceutically acceptable salt thereof, a prodrug thereof, or a polymorph thereof. A prodrug thereof is for example sergliflozin etabonate (CAS: 408504-26-7; ethyl [(2R,3S,4S,5R,6S)-3,4,5-trihydroxy-6-[2-[(4-methoxyphenyl)methyl]phenoxy]oxan-2-yl]methyl carbonate). “Sergliflozin” or “sergliflozin etabonate” are also known under the following terms: (2R,3S,4S,5R,6S)-2-(hydroxymethyl)-6-[2-[(4-methoxyphenyl)methyl]phenoxy]oxane-3,4,5-triol, 2-[(4-methoxyphenyl) methylphenyl 6-O-(ethoxycarbonyl)-β-D-glucopyranoside, sergliflozin etabonate, SERGLIFLOZIN A, UNII-AR34521QFL, AR34521QFL, CHEMBL270766, β-glucoside, 4a, SCHEMBL2574820, BDBM20878, beta-D-Glucopyranoside, 2-((4-methoxyphenyl)methyl)phenyl, HFLCZNNDZKKXCS-OUUBHVDSSA-N, J3.551.075C, ethyl [(2R,3S,4S,5R,6S)-3,4,5-trihydroxy-6-[2-[(4-methoxyphenyl)methyl]phenoxy]oxan-2-yl]methyl carbonate, UNII-4HY3523466, GW869682X, 4HY3523466, 2-(4-Methoxybenzyl)phenyl 6-O-(ethoxycarbonyl)-beta-D-glucopyranoside, CHEMBL450044. The list is not exhaustive as there a several marketed product on the market comprising “sergliflozin” or further synonyms, which are included in the term. In the following, when referring herein to “sergliflozin”, all the aforementioned synonyms/terms are meant.

In one embodiment the SGLT2 inhibitor is sotagliflozin. The term “compound according to formula (IX)” or “sotagliflozin” or “(2S,3R,4R,5S,6R)-2-[4-chloro-3-[(4-ethoxyphenyl)methyl]phenyl]-6-methylsulfanyloxane-3,4,5-triol” (CAS number: 1018899-04-1) refers to an anhydrate thereof, hydrate thereof, solvate thereof, pharmaceutically acceptable salt thereof, a prodrug thereof, or a polymorph thereof “Sotagliflozin” is also known under the following terms: (2S,3R,4R,5S,6R)-2-(4-chloro-3-(4-ethoxybenzyl)phenyl)-6-(methylthio)tetrahydro-2H-pyran-3,4,5-triol, Zynquista® (trade name), LX-4211 and LX4211. The list is not exhaustive as there a several marketed product on the market comprising “sotagliflozin” or further synonyms, which are included in the term. In the following, when referring herein to “sotagliflozin”, all the aforementioned synonyms/terms are meant.

In one embodiment the SGLT2 inhibitor is tofogliflozin”. The term “compound according to formula (X)” or “tofogliflozin” or “(3S,3′R,4'S,5'S,6′R)-5-[(4-ethylphenyl)methyl]-6′-(hydroxymethyl)spiro[1H-2-benzofuran-3,2′-oxane]-3′,4′,5′-triol” refers to an anhydrate thereof, hydrate thereof, solvate thereof, pharmaceutically acceptable salt thereof, a prodrug thereof, or a polymorph thereof. The CAS number of the hydrate is 1201913-82-7 and number of the anhydrate is 903565-83-3. The United States Adopted Name tofogliflozin applies to the monohydrate, which is the form used as a drug. The International Nonproprietary Name tofogliflozin applies to the anhydrous compound and the drug form is referred to as tofogliflozin hydrate. “Tofogliflozin” is also known under the following terms: 6-((4-ethylphenyl)methyl)-3′,4′,5′,6′-tetrahydro-6′-(hydroxymethyl)spiro(isobenzofuran-1(3H),2′-(2H)pyran)-3′,4′,5′-triol, (3S,3′R,4'S,5'S,6′R)-5-[(4-ethylphenyl)methyl]-6′-(hydroxymethyl)spiro[1H-2-benzofuran-3,2′-oxane]-3′,4′,5′-triol, Apleway, CSG452, Deberza, tofogliflozin anhydrous, or tofogliflozin hydrate. The list is not exhaustive as there a several marketed product on the market comprising “tofogliflozin” or further synonyms, which are included in the term. In the following, when referring herein to “tofogliflozin”, all the aforementioned synonyms/terms are meant.

“Solvates” for the purposes of the invention are those forms of the compounds or their salts where solvent molecules form a stoichiometric complex in the solid state and include, but are not limited to for example water, ethanol and methanol.

“Hydrates” are a specific form of solvates, where the solvent molecule is water. Hydrates of the compounds of the invention or their salts are stoichiometric compositions of the compounds or salts with water, such as, for example, monohydrate, dihydrates, trihydrate, hemihydrate, sequihydrate.

“Salts” for the purposes of the present invention are preferably “pharmaceutically acceptable salts” of finerenone and/or a SGLT2 inhibitor. Suitable pharmaceutically acceptable salts that can be used in the combination according to the invention are well known to those skilled in the art and include salts of inorganic acids, organic acids, inorganic bases, alkaline cations, alkaline earth cations and organic bases. In one embodiment the pharmaceutically acceptable salt can be selected from hydrochloric acid, hydrobromic acid, sulfuric acid, phosphoric acid, methane sulphonic acid, trifluoromethanesulfonic acid, benzenesulfonic acid, p-toluene sulfonic acid, 1-naphthalenesulfonic acid, 2-naphthalenesulfonic acid, acetic acid, trifluoroacetic acid, malic acid, tartaric acid, citric acid, lactic acid, oxalic acid, succinic acid, fumaric acid, maleic acid, benzoic acid, salicylic acid, phenylacetic acid, and mandelic acid acetate, benzoate, besylate, bromide, camsylate, carbonate, citrate, edisylate, estolate, fumarate, gluceptate, gluconate, glucuronate, hippurate, iodide, isethionate, lactate, lactobionate, malate, maleate, mesylate, methylsulfate, napsylate, nitrate, oxalate, pamoate, phosphate, stearate, succinate, sulfate, tartrate, bitartrate, tosylate, calcium, diolamine, lithium, lysine, magnesium, meglumine, N-methylglucamine, olamine, potassium, tromethamine, tris(hydroxymethyl)aminomethane, benzenesulfonate, ethanesulfonate and zinc.

In one embodiment the pharmaceutically acceptable salt can be selected from hydrochloride, sulfate, mesylate, tosylate, tartrate, citrate, benzenesulfonate, ethanesulfonate, maleate, and phosphate.

The term “combination” in the present invention is used as known to persons skilled in the art. The combination comprises or consists of finerenone or a hydrate, solvate, pharmaceutically acceptable salt thereof, or a polymorph thereof, and a SGLT2 inhibitor a hydrate, solvate, pharmaceutically acceptable salt thereof, or a polymorph thereof “Combination” for the purposes of the invention comprises fixed combinations, combination packs, kit-of-parts, non-fixed combinations and/or components (finerenone, a SGLT2 inhibitor), which are administered simultaneously or sequentially. The term “the components” refers to finerenone or a hydrate, solvate, pharmaceutically acceptable salt thereof, or a polymorph thereof, and a SGLT2 inhibitor a hydrate, solvate, pharmaceutically acceptable salt thereof, i.e. one component of the combination according to the invention is finerenone or a hydrate, solvate, pharmaceutically acceptable salt thereof, or a polymorph thereof and the other component is a SGLT2 inhibitor a hydrate, solvate, pharmaceutically acceptable salt thereof to. The combination according to the invention can also comprise further components.

In one embodiment according to the invention, the combination is a fixed combination. A “fixed combination” in the present invention is used as known to persons skilled in the art. It is defined as a combination comprising or containing finerenone and a SGLT2 inhibitor within one pharmaceutical dosage form or in one single entity. One example of a “fixed combination” is a pharmaceutical composition, wherein finerenone and a SGLT2 inhibitor are present in admixture. Another example of a “fixed combination” is a pharmaceutical combination, wherein finerenone and a SGLT2 inhibitor are present in one dosage form without being in admixture. Another example of a “fixed combination” is a pharmaceutical combination, wherein finerenone and a SGLT2 inhibitor are present at least partially admixture.

“Pharmaceutical dose form” and “dosage form” are synonyms. “Pharmaceutical dose form” or “dosage form” is the physical manifestation of a product that contains or comprises the active ingredient and/or inactive ingredients (e.g. carrier, excipients) that are intended to be delivered to the patient. “Dosage form” is the term used in the European Pharmacopoeia. “Dosage form” was previously used in Standard Terms, but the term “pharmaceutical dose form” is now used in order to harmonise with the vocabulary that is used across the Identification of Medicinal Products project (cf. https://www.edgm.eu/sites/default/files/standard_terms_introduction_and_guidance_for_use.pdf). Common dosage forms include pill, tablet, capsule, syrup, aerosol, liquid injection, powder, or solid crystal, and so on. Further pharmaceutical formulations or dosage forms are disclosed below. The route of administration for drug delivery is dependent on the dosage form of the active ingredient.

In one embodiment according to the invention, the combination is a combined pharmaceutical dose form. The “combined pharmaceutical dose form” is used to combine two or more pharmaceutical dose forms into a single term, in order to describe a medicinal product that consists of two or more manufactured items that are intended to be combined to produce a single pharmaceutical product for administration to the patient. A combined pharmaceutical dose form is not used to combine pharmaceutical dose forms that are packaged together, but administered separately rather than being combined to produce a single pharmaceutical product (see instead combination packs). In one embodiment the combined pharmaceutical dose form comprises a soluble powder, tablet or granulate, which comprises finerenone and/or a SGLT2 inhibitor, and a solvent for solving or dispersing the powder, tablet or granulate. The combined pharmaceutical dose form is not limited to this exemplary embodiment. Various combinations of the dosage forms disclosed herein are suitable for the combination according to the invention.

In one embodiment according to the invention, the combination is a non-fixed combination or kit-of-parts. A “non-fixed combination” or “kit-of-parts” in the present invention is used as known to persons skilled in the art and is defined as a combination, wherein finerenone and a SGLT2 inhibitor are present in more than one unit or dosage form. It is possible for the components of the non-fixed combination or kit-of-parts to be administered separately, sequentially, simultaneously, concurrently or chronologically staggered. In one embodiment of a non-fixed combination or kit-of-parts finerenone and a SGLT2 inhibitor are present separately. In one embodiment the non-fixed combination or kit-of-parts comprises a pharmaceutical composition comprising finerenone and a pharmaceutical composition comprising a SGLT2 inhibitor. In one embodiment of a non-fixed combination or kit-of-parts comprises a dosage form comprising finerenone and a dosage form comprising a SGLT2 inhibitor. The non-fixed combination or kit-of-parts is not limited to this exemplary embodiment(s). Various combinations of the dosage forms disclosed herein are suitable for the combination according to the invention.

The non-fixed combination or kit-of-parts is particularly advantageous if the separate components have to be administered in different dose forms or are administered in different dose intervals.

In one embodiment, the combination according to the invention is a combination pack.

In a “combination pack” active substances are included in separate dosage forms marketed in the same package. The combination pack is used to combine two or more dose forms to describe products that are packaged together but are administered separately, sequentially, simultaneously, concurrently or chronologically staggered as independent pharmaceutical products. A combination is different from a combined pharmaceutical dose form. In one embodiment, the combination pack comprises a first dosage form comprising finerenone and a second dosage form comprising a SGLT2 inhibitor. In one embodiment the combination pack comprises first oral dosage form comprising finerenone and a second oral dosage form comprising a SGLT2 inhibitor. The oral dosage form can be selected from the oral dosage form and in any combination described herein. In one embodiment the combination pack comprises a tablet comprising finerenone and a granulate for al solution comprising a SGLT2 inhibitor vice versa. In this embodiment the combination comprises a tablet as administrable dose form and oral solution as administrable dose form.

In one embodiment the combinations according to the invention, finerenone and a SGLT2 inhibitor are administered successively or separately. In the combinations according to the invention, the compounds are administered in at least two separate dosage forms.

In one embodiment, the separate dosage forms are identical. In one example of this embodiment, the combination comprises a first tablet comprising finerenone and a second tablet comprising a SGLT2 inhibitor (herein, the separate dosage form are identical as both dosage forms are tablets). This embodiment is not limited to the combination of two tablets. Further dosage forms, which can be used in this embodiment are disclosed below. This embodiment, wherein separate dosage forms are identical, can be used for example in a combined pharmaceutical dose form, non-fixed-combination, kit-of-parts and/or combination pack.

In one embodiment, the separate dosage forms are different. In one example of this embodiment, the combination comprises a tablet comprising finerenone and a capsule comprising a SGLT2 inhibitor (herein, the separate dosage form are different as the dosage forms differ from each other). This embodiment is not limited to the combination of a tablet and capsule. Further dosage forms, which can be used in this embodiment are disclosed below. This embodiment, wherein separate dosage forms are different, can be used for example in a combined pharmaceutical dose form, non-fixed-combination, kit-of-parts and/or combination pack.

The release of one or more agents of the combination can also be controlled, where appropriate, to provide the desired therapeutic activity when in a single dosage form, combination pack, kit-of-parts or when in separate independent dosage forms.

In one embodiment the combination according to the invention is administered one or more times per day. The combination according to the invention can be applied once daily (once daily administration). In one embodiment administration is performed via the oral route once or more time per day. The combination according to the invention can also comprise two separate dosage forms, wherein the respective dosage form is given at a different time point.

The present invention includes pharmaceutical compositions or dosage forms which are comprised of a pharmaceutically effective amount of a SGLT2 inhibitor and finerenone and pharmaceutically acceptable carrier and/or excipient. A “pharmaceutically effective amount” of finerenone or a SGLT2 inhibitor (active ingredients) is that amount which produces a result or exerts an influence on the particular condition being treated. A “pharmaceutically acceptable carrier” is any carrier which is relatively non-toxic and innocuous to a patient at concentrations consistent with effective activity of the active ingredient so that any side effects ascribable to the carrier do not vitiate the beneficial effects of the active ingredient. An “excipient” is used as known in the art. It is a substance formulated alongside the active ingredient of a medication, included for e.g. the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity, or enhancing solubility. Excipients can also be useful in the manufacturing process, to aid in the handling of the active substance concerns such as by facilitating powder flowability or non-stick properties, in addition to aiding in vitro stability such as prevention of denaturation or aggregation over the expected shelf life. The selection of appropriate excipients also depends upon the route of administration and the dosage form, as well as the active ingredient and other factors. Some excipients are described below.

Commonly used pharmaceutical ingredients which can be used as appropriate to formulate the composition for its intended route of administration include:
acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃);
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);
chelating agents (examples include but are not limited to edetate disodium and edetic acid);
colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogol, cetyl alcohol, glyceryl monostearate, lecithin, sorbitan monooleate, polyoxyethylene 50 monostearate);
encapsulating agents (examples include but are not limited to gelatin and cellulose acetate phthalate);
flavorants (examples include but are not limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas);
plasticizers (examples include but are not limited to diethyl phthalate and glycerol);
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnauba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithin, sorbitol monooleate, polyoxyethylene sorbitol monooleate, and polyoxyethylene stearate).

It is believed that one skilled in the art, utilizing the preceding information, can utilize the present invention to its fullest extent.

Combinations of the present invention can be administered in any form by any effective route, including, for example oral, parenteral, enteral, intravenous, intraperitoneal, topical, transdermal (for example using any standard patch), ophthalmic, nasally, local, non-oral, such as aerosol, inhalation, subcutaneous, intramuscular, buccal, sublingual, rectal, vaginal, intra-arterial, and intrathecal, etc. They can be administered alone, or in combination with any ingredient(s), active or inactive.

Combinations of the present invention can be converted in a known manner into the usual dosage forms, pharmaceutical dose form or pharmaceutical formulations, which can be liquid or solid formulations for example without limitation tablets, coated tablets, capsules, pills, powders, granules, elixirs, tinctures, solution, suspensions, syrups, solid and liquid aerosols and emulsions.

In one embodiment the combination according to the invention is administered via oral administration. In one embodiment the combination according to the invention is comprised in one dosage form for oral administration. For oral administration, the compounds can be formulated into solid or liquid can be prepared according to methods known to the art for the manufacture of pharmaceutical compositions. Examples of dosage forms for al administration of finerenone or a SGLT2 inhibitors are described in WO 2008/104306 A1 (see WO 2008/104306 A1, section C) and WO 2016/071212 A1 (see WO 2016/071212 A1, section C) as well as in the Examples of the present application in section A.1, “Tablet comprising finerenone” below.

In one embodiment the combination according to the invention is comprised in one dosage form. In one embodiment the combination according to the invention consists of two separate dosage form. In one embodiment the combination according to the invention consists of two separate dosage form for oral administration.

In one embodiment the combination according to the invention is a capsule. In one embodiment the combination according to the invention is a tablet. In one embodiment the combination according to the invention consists of one single tablet comprising finerenone and a SGLT2 inhibitor. In one embodiment the combination according to the invention comprises at least two tablets, wherein one tablet comprises finerenone and the other tablet comprises a SGLT2 inhibitor. In one embodiment the combination according to the invention comprises at least two tablets, wherein one tablet comprises finerenone, but no SGLT2 inhibitor, and the other tablet comprises a SGLT2 inhibitor, but no finerenone.

In another embodiment, finerenone and/or a SGLT2 inhibitor to the invention can be tableted with conventional tablet bases such as lactose, sucrose and corn starch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials can be present as coatings or to otherwise modify the physical form of the dosage form. For instance tablets, pills or capsules can be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase can be a vegetable oil such as liquid paraffin or a mixture of vegetable oils. Suitable emulsifying agents can be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions can be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs can be formulated with sweetening agents such as, for example, glycerol, propylene glycol, sorbitol or sucrose. Such formulations may also contain a demulcent, and preservative, such as methyl and propyl parabens and flavoring and coloring agents.

The pharmaceutical compositions of this invention may also be in the form of a dispersion. A “dispersion” is a system in which distributed particles of one material are dispersed in a continuous phase (sometimes called matrix) of another material. The two phases may be in the same or different states of matter. Dispersions are classified in a number of different ways, including how large the particles are in relation to the particles of the continuous phase, whether or not precipitation occurs, and the presence of Brownian motion. In general, dispersions of particles sufficiently large for sedimentation are called suspensions, while those of smaller particles are called colloids and solutions.

The pharmaceutical compositions of this invention may also be in the form of a solid dispersion. The solid dispersion can be a solid solution, glass solution, glass suspension, amorphous precipitation in a crystalline carrier, eutectic or monotecic, compound or complex formation and combinations thereof.

An aspect of the invention of particular interest is a pharmaceutical composition comprising a solid dispersion, wherein the matrix comprises a pharmaceutically acceptable carrier is a polymer, such as polyvinylpyrrolidone, vinylpyrrolidone/vinylacetate copolymer, polyalkylene glycol (i.e. polyethylene glycol), hydroxyalkyl cellulose (i.e. hydroxypropyl cellulose), hydroxyalkyl methyl cellulose (i.e. hydroxypropyl methyl cellulose), carboxymethyl cellulose, sodium carboxymethyl cellulose, ethyl cellulose, polymethacrylates, polyvinyl alcohol, polyvinyl acetate, vinyl alcohol/vinyl acetate copolymer, polyglycolized glycerides, xanthan gum, carrageenan, chitosan, chitin, poyldextrin, dextrin, starch and proteins.

Another aspect of the invention is a pharmaceutical composition comprising a solid dispersion, wherein the matrix comprises a sugar and/or sugar alcohol and/or cyclodextrin, for example sucrose, lactose, fructose, maltose, raffinose, sorbitol, lactitol, mannitol, maltitol, erythritol, inositol, trehalose, isomalt, inulin, maltodextrin, β-cyclodextrin, hydroxypropyl-β-cyclodextrin or sulfobutyl ether cyclodextrin.

Additional suitable carriers that are useful in the formation of the matrix of the solid dispersion include, but are not limited to alcohols, organic acids, organic bases, amino acids, phospholipids, waxes, salts, fatty acid esters, polyoxyethylene sorbitan fatty acid esters, and urea.

The solid dispersion of finerenone in the matrix may contain certain additional pharmaceutical acceptable ingredients.

The solid dispersion of the invention is prepared according to methods known to the art for the manufacture of solid dispersions, such as fusion/melt technology, hot melt extrusion, solvent evaporation (i.e. freeze drying, spray drying or layering of powders of granules), coprecipitation, supercritical fluid technology and electrostatic spinning method.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

The amount of the administered active ingredient can vary widely according to such considerations as the particular compound and dosage form employed, the mode and time of administration, the period of treatment, the age, sex, and general condition of the patient treated, the nature and extent of the condition treated, the rate of drug metabolism and excretion, the potential drug combinations and drug-drug interactions, and the like.

One embodiment according to the invention refers to the combination comprising finerenone in an amount of 0.25 mg to 80 mg. One embodiment according to the invention refers to the combination comprising finerenone in an amount of 0.25 mg to 40 mg. One embodiment according to the invention refers to the combination comprising finerenone in an amount of 0.25 mg to 20 mg. One embodiment according to the invention refers to the combination comprising finerenone in an amount of 0.25 mg to 10 mg. One embodiment according to the invention refers to the combination comprising finerenone in an amount of 0.25 mg to 5 mg.

One embodiment according to the invention refers to the combination comprising finerenone in an amount of 5 to 80 mg. In one embodiment the combination according to the invention comprises finerenone in an amount of 5 to 80 mg, 5 to 70 mg, 5 to 60 mg, 5 to 50 mg, 5 to 40 mg, 10 to 80 mg, 10 to 70 mg, 10 to 60 mg, 10 to 50 mg or 10 to 40 mg. In one embodiment the combination according to the invention comprises finerenone in an amount of 10 to 40 mg. In one embodiment the combination according to the invention comprises finerenone in an amount of 20 to 40 mg. In one embodiment the combination according to the invention comprises finerenone in an amount of 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 55, 60, 65, 70, 75 or 80 mg. In one embodiment the combination according to the invention comprises finerenone in an amount of 5, 10, 15, 20, 25, 30, 35, 40, 45, 50, 55, 60, 65, 70, 75 or 80 mg. In one embodiment the combination according to the invention comprises finerenone in an amount of 5 mg. In one embodiment the combination according to the invention comprises finerenone in an amount of 10 mg. In one embodiment the combination according to the invention comprises finerenone in an amount of 15 mg. In one embodiment the combination according to the invention comprises finerenone in an amount of 20 mg. In one embodiment the combination according to the invention comprises finerenone in an amount of 25 mg. In one embodiment the combination according to the invention comprises finerenone in an amount of 30 mg. In one embodiment the combination according to the invention comprises finerenone in an amount of 35 mg. In one embodiment the combination according to the invention comprises finerenone in an amount of 40 mg. In one embodiment the combination according to the invention comprises finerenone in an amount of 5 mg, 10 mg, 15 mg, 20 mg, 25 mg, 30 mg, 35 mg or 40 mg. In one embodiment the combination according to the invention comprises finerenone in an amount of 5 mg, 10 mg, 20 mg, 30 mg or 40 mg.

One embodiment according to the invention refers to the combination comprising a SGLT2 inhibitor in an amount of 0.5 to 400 mg. In one embodiment the combination according to the invention comprises a SGLT2 inhibitor in an amount of 0.5 to 300 mg, 1 to 300 mg, 2 to 200 mg, 3 to 100 mg, 5 to 100 mg, 5 to 90 mg, 5 to 80 mg, 5 to 70 mg, 5 to 60 mg, 5 to 50 mg, 5 to 40 mg, 5 to 30 mg 10 to 90 mg, 10 to 80 mg, 10 to 70 mg, 10 to 60 mg, 10 to 50 mg or 10 to 40 mg, 10 to 30 mg 15 to 90 mg, 15 to 80 mg, 15 to 70 mg, 15 to 60 mg, 15 to 50 mg, 15 to 40 mg or 15 to 30 mg.

In one embodiment the combination according to the invention comprises a SGLT2 inhibitor in an amount of 1 to 20 mg. In one embodiment the combination according to the invention comprises a SGLT2 inhibitor in an amount of 3 to 15 mg. In one embodiment the combination according to the invention comprises a SGLT2 inhibitor in an amount of 5 to 10 mg.

In one embodiment the combination according to the invention comprises a SGLT2 inhibitor in an amount of 1, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290 or 300 mg.

In one embodiment the combination according to the invention comprises a SGLT2 inhibitor in an amount of 3 mg. In one embodiment the combination according to the invention comprises a SGLT2 inhibitor in an amount of 5 mg. In one embodiment the combination according to the invention comprises a SGLT2 inhibitor in an amount of 10 mg. In one embodiment the combination according to the invention comprises a SGLT2 inhibitor in an amount of 15 mg. In one embodiment the combination according to the invention comprises a SGLT2 inhibitor in an amount of 20 mg. In one embodiment the combination according to the invention comprises a SGLT2 inhibitor in an amount of 25 mg. In one embodiment the combination according to the invention comprises a SGLT2 inhibitor in an amount of 30 mg. combination according to the invention can comprise a SGLT2 inhibitor in an amount of 35 mg. In one embodiment the combination according to the invention comprises a SGLT2 inhibitor in an amount of 40 mg. In one embodiment the combination according to the invention comprises a SGLT2 inhibitor in an amount of 50 mg. In one embodiment the combination according to the invention comprises a SGLT2 inhibitor in an amount of 60 mg. In one embodiment the combination according to the invention comprises a SGLT2 inhibitor in an amount of 65 mg. In one embodiment the combination according to the invention comprises a SGLT2 inhibitor in an amount of 70 mg. In one embodiment the combination according to the invention comprises a SGLT2 inhibitor in an amount of 80 mg. In one embodiment the combination according to the invention comprises a SGLT2 inhibitor in an amount of 90 mg. In one embodiment the combination according to the invention comprises a SGLT2 inhibitor in an amount of 95 mg. In one embodiment the combination according to the invention comprises a SGLT2 inhibitor in an amount of 100 mg. In one embodiment the combination according to the invention comprises a SGLT2 inhibitor in an amount of 125 mg. In one embodiment the combination according to the invention comprises a SGLT2 inhibitor in an amount of 150 mg. In one embodiment the combination according to the invention comprises a SGLT2 inhibitor in an amount of 175 mg. In one embodiment the combination according to the invention comprises a SGLT2 inhibitor in an amount of 200 mg. In one embodiment the combination according to the invention comprises a SGLT2 inhibitor in an amount of 300 mg.

In one embodiment the combination according to the invention comprises finerenone in an amount 5 to 80 mg and a SGLT2 inhibitor in an amount of 0.5 to 300 mg. In one embodiment the combination according to the invention comprises finerenone in an amount 10 to 40 mg and a SGLT2 inhibitor in an amount of 0.5 to 300 mg.

In one embodiment the combination according to the invention comprises finerenone in an amount 5 to 80 mg and a SGLT2 inhibitor in an amount of 3 to 300 mg. In one embodiment the combination according to the invention comprises finerenone in an amount 10 to 40 mg and a SGLT2 inhibitor in an amount of 3 to 300 mg.

In one embodiment the combination according to the invention comprises finerenone in an amount 5 to 80 mg and a SGLT2 inhibitor in an amount of 3 to 200 mg. In one embodiment the combination according to the invention comprises finerenone in an amount 10 to 40 mg and a SGLT2 inhibitor in an amount of 3 to 200 mg.

In one embodiment the combination according to the invention comprises finerenone in an amount 5 to 80 mg and a SGLT2 inhibitor in an amount of 3 to 150 mg. In one embodiment the combination according to the invention comprises finerenone in an amount 10 to 40 mg and a SGLT2 inhibitor in an amount of 3 to 150 mg.

In one embodiment the combination according to the invention comprises finerenone in an amount 5 to 80 mg and a SGLT2 inhibitor in an amount of 3 to 100 mg. In one embodiment the combination according to the invention comprises finerenone in an amount 10 to 40 mg and a SGLT2 inhibitor in an amount of 3 to 100 mg.

In one embodiment the combination according to the invention comprises finerenone in an amount 5 to 80 mg and a SGLT2 inhibitor in an amount of 5 to 100 mg. In one embodiment the combination according to the invention comprises finerenone in an amount 10 to 40 mg and a SGLT2 inhibitor in an amount of 5 to 100 mg.

In one embodiment the combination according to the invention comprises finerenone in an amount 5 to 80 mg and a SGLT2 inhibitor in an amount of 3 to 10 mg. In one embodiment the combination according to the invention comprises finerenone in an amount 10 to 40 mg and a SGLT2 inhibitor in an amount of 3 to 25 mg.

In one embodiment the combination according to the invention comprises finerenone in an amount 5 to 80 mg and a SGLT2 inhibitor in an amount of 3 to 10 mg. In one embodiment the combination according to the invention comprises finerenone in an amount 10 to 40 mg and a SGLT2 inhibitor in an amount of 3 to 15 mg.

In one embodiment the combination according to the invention comprises finerenone in an amount 5 to 80 mg and a SGLT2 inhibitor in an amount of 3 to 10 mg. In one embodiment the combination according to the invention comprises finerenone in an amount 10 to 40 mg and a SGLT2 inhibitor in an amount of 3 to 10 mg.

In one embodiment the combination according to the invention comprises finerenone in an amount 5 to 80 mg and a SGLT2 inhibitor in an amount of 5 to 10 mg. In one embodiment the combination according to the invention comprises finerenone in an amount 10 to 40 mg and a SGLT2 inhibitor in an amount of 5 to 10 mg.

In one embodiment the combination according to the invention comprises finerenone in an amount 5 to 80 mg and a SGLT2 inhibitor in an amount of 100 mg. In one embodiment the combination according to the invention comprises finerenone in an amount 10 to 40 mg and a SGLT2 inhibitor in an amount of 100 mg.

In one embodiment the combination according to the invention comprises finerenone in an amount 10 mg and a SGLT2 inhibitor in an amount of 3 mg. In one embodiment the combination according to the invention comprises finerenone in an amount 10 mg and a SGLT2 inhibitor in an amount of 5 mg. In one embodiment the combination according to the invention comprises finerenone in an amount 10 mg and a SGLT2 inhibitor in an amount of 10 mg. In one embodiment the combination according to the invention comprises finerenone in an amount 10 mg and a SGLT2 inhibitor in an amount of 100 mg.

In one embodiment the combination according to the invention comprises finerenone in an amount 20 mg and a SGLT2 inhibitor in an amount of 3 mg In one embodiment the combination according to the invention comprises finerenone in an amount 20 mg and a SGLT2 inhibitor in an amount of 5 mg. In one embodiment the combination according to the invention comprises finerenone in an amount 20 mg and a SGLT2 inhibitor in an amount of 10 mg. In one embodiment the combination according to the invention comprises finerenone in an amount 20 mg and a SGLT2 inhibitor in an amount of 100 mg.

In one embodiment the combination according to the invention comprises finerenone in an amount 30 mg and a SGLT2 inhibitor in an amount of 3 mg. In one embodiment the combination according to the invention comprises finerenone in an amount 30 mg and a SGLT2 inhibitor in an amount of 5 mg. In one embodiment the combination according to the invention comprises finerenone in an amount 30 mg and a SGLT2 inhibitor in an amount of 10 mg. In one embodiment the combination according to the invention comprises finerenone in an amount 30 mg and a SGLT2 inhibitor in an amount of 100 mg.

In one embodiment the combination according to the invention comprises finerenone in an amount 40 mg and a SGLT2 inhibitor in an amount of 3 mg. In one embodiment the combination according to the invention comprises finerenone in an amount 40 mg and a SGLT2 inhibitor in an amount of 5 mg. In one embodiment the combination according to the invention comprises finerenone in an amount 40 mg and a SGLT2 inhibitor in an amount of 10 mg. In one embodiment the combination according to the invention comprises finerenone in an amount 40 mg and a SGLT2 inhibitor in an amount of 100 mg.

In one embodiment the combination according to the invention comprises finerenone and canagliflozin in an amount of 5 to 400 mg. In one embodiment the combination according to the invention comprises finerenone and canagliflozin in an amount of 5 to 300 mg. In one embodiment the combination according to the invention comprises finerenone and canagliflozin in an amount of 5 to 150 mg. In one embodiment the combination according to the invention comprises finerenone and canagliflozin in an amount of 50 to 150 mg. In one embodiment the combination according to the invention comprises finerenone and canagliflozin in an amount of 90 to 110 mg. In one embodiment the combination according to the invention comprises finerenone and canagliflozin in an amount of 100 mg. In one embodiment the combination according to the invention comprises finerenone and canagliflozin in an amount of 150 mg. In one embodiment the combination according to the invention comprises finerenone and canagliflozin in an amount of 300 mg.

In one embodiment the combination according to the invention comprises finerenone and canagliflozin in an amount of 5, 10, 20, 30, 30, 40, 50, 60, 70, 80, 90, 100, 110, 120, 130, 140, 150, 160, 170, 180, 190, 200, 210, 220, 230, 240, 250, 260, 270, 280, 290 or 300 mg.

In one embodiment the combination according to the invention comprises finerenone in an amount of 5, 10, 15, 20, 25, 30, 35, 40, 45, 50, 55, 60, 65, 70, 75 or 80 mg and canagliflozin.

In one embodiment the combination according to the invention comprises finerenone in an amount of 5 to 80 mg and canagliflozin in an amount of 5 to 150 mg. In one embodiment the combination according to the invention comprises finerenone in an amount of 10 to 40 mg and canagliflozin in an amount of 5 to 150 mg.

In one embodiment the combination according to the invention comprises finerenone in an amount of 5 to 80 mg and canagliflozin in an amount of 100 mg. In one embodiment the combination according to the invention comprises finerenone in an amount of 10 to 40 mg and canagliflozin in an amount of 100 mg.

In one embodiment the combination according to the invention comprises finerenone in an amount of 5 mg and canagliflozin in an amount of 100 mg. In one embodiment the combination according to the invention comprises finerenone in an amount of 10 mg and canagliflozin in an amount of 100 mg. In one embodiment the combination according to the invention comprises finerenone in an amount of 20 mg and canagliflozin in an amount of 100 mg. In one embodiment the combination according to the invention comprises finerenone in an amount of 30 mg and canagliflozin in an amount of 100 mg. In one embodiment the combination according to the invention comprises finerenone in an amount of 40 mg and canagliflozin in an amount of 100 mg. In one embodiment the combination according to the invention comprises finerenone in an amount of 50 mg and canagliflozin in an amount of 100 mg. In one embodiment the combination according to the invention comprises finerenone in an amount of 60 mg and canagliflozin in an amount of 100 mg. In one embodiment the combination according to the invention comprises finerenone in an amount of 70 mg and canagliflozin in an amount of 100 mg. In one embodiment the combination according to the invention comprises finerenone in an amount of 80 mg and canagliflozin in an amount of 100 mg.

In one embodiment the combination according to the invention comprises finerenone in an amount of 5 mg and canagliflozin in an amount of 200 mg. In one embodiment the combination according to the invention comprises finerenone in an amount of 10 mg and canagliflozin in an amount of 200 mg. In one embodiment the combination according to the invention comprises finerenone in an amount of 20 mg and canagliflozin in an amount of 200 mg. In one embodiment the combination according to the invention comprises finerenone in an amount of 30 mg and canagliflozin in an amount of 200 mg. In one embodiment the combination according to the invention comprises finerenone in an amount of 40 mg and canagliflozin in an amount of 200 mg. In one embodiment the combination according to the invention comprises finerenone in an amount of 50 mg and canagliflozin in an amount of 200 mg. In one embodiment the combination according to the invention comprises finerenone in an amount of 60 mg and canagliflozin in an amount of 200 mg. In one embodiment the combination according to the invention comprises finerenone in an amount of 70 mg and canagliflozin in an amount of 200 mg. In one embodiment the combination according to the invention comprises finerenone in an amount of 80 mg and canagliflozin in an amount of 200 mg.

In one embodiment the combination according to the invention comprises finerenone in an amount of 5 mg and canagliflozin in an amount of 300 mg. In one embodiment the combination according to the invention comprises finerenone in an amount of 10 mg and canagliflozin in an amount of 300 mg. In one embodiment the combination according to the invention comprises finerenone in an amount of 20 mg and canagliflozin in an amount of 300 mg. In one embodiment the combination according to the invention comprises finerenone in an amount of 30 mg and canagliflozin in an amount of 300 mg. In one embodiment the combination according to the invention comprises finerenone in an amount of 40 mg and canagliflozin in an amount of 300 mg. In one embodiment the combination according to the invention comprises finerenone in an amount of 50 mg and canagliflozin in an amount of 300 mg. In one embodiment the combination according to the invention comprises finerenone in an amount of 60 mg and canagliflozin in an amount of 300 mg. In one embodiment the combination according to the invention comprises finerenone in an amount of 70 mg and canagliflozin in an amount of 300 mg. In one embodiment the combination according to the invention comprises finerenone in an amount of 80 mg and canagliflozin in an amount of 300 mg.

In one embodiment the combination according to the invention comprises finerenone and dapagliflozin in an amount of 0.5 to 20 mg. In one embodiment the combination according to the invention comprises finerenone and dapagliflozin in an amount of 3 to 15 mg. In one embodiment the combination according to the invention comprises finerenone and dapagliflozin in an amount of 5 to 10 mg. In one embodiment the combination according to the invention comprises finerenone and dapagliflozin in an amount of 5 mg. In one embodiment the combination according to the invention comprises finerenone and dapagliflozin in an amount of 10 mg.

In one embodiment the combination according to the invention comprises finerenone and dapagliflozin in an amount of 0.5, 1, 2, 3, 4, 5, 6, 7, 8, 9 or 10 mg.

In one embodiment the combination according to the invention comprises finerenone in an amount of 5, 10, 15, 20, 25, 30, 35, 40, 45, 50, 55, 60, 65, 70, 75 or 80 mg and dapagliflozin.

In one embodiment the combination according to the invention comprises finerenone in an amount of 5 to 80 mg and dapagliflozin in an amount of 0.5 to 20 mg. In one embodiment the combination according to the invention comprises finerenone in an amount of 10 to 40 mg and dapagliflozin in an amount of 0.5 to 20 mg.

In one embodiment the combination according to the invention comprises finerenone in an amount of 5 to 80 mg and dapagliflozin in an amount of 5 mg. In one embodiment the combination according to the invention comprises finerenone in an amount of 10 to 40 mg and dapagliflozin in an amount of 5 mg.

In one embodiment the combination according to the invention comprises finerenone in an amount of 5 mg and dapagliflozin in an amount of 5 mg. In one embodiment the combination according to the invention comprises finerenone in an amount of 10 mg and dapagliflozin in an amount of 5 mg. In one embodiment the combination according to the invention comprises finerenone in an amount of 20 mg and dapagliflozin in an amount of 5 mg. In one embodiment the combination according to the invention comprises finerenone in an amount of 30 mg and dapagliflozin in an amount of 5 mg. In one embodiment the combination according to the invention comprises finerenone in an amount of 40 mg and dapagliflozin in an amount of 5 mg. In one embodiment the combination according to the invention comprises finerenone in an amount of 50 mg and dapagliflozin in an amount of 5 mg. In one embodiment the combination according to the invention comprises finerenone in an amount of 60 mg and dapagliflozin in an amount of 5 mg. In one embodiment the combination according to the invention comprises finerenone in an amount of 70 mg and dapagliflozin in an amount of 5 mg. In one embodiment the combination according to the invention comprises finerenone in an amount of 80 mg and dapagliflozin in an amount of 5 mg.

In one embodiment the combination according to the invention comprises finerenone in an amount of 5 to 80 mg and dapagliflozin in an amount of 0.5 to 20 mg. In one embodiment the combination according to the invention comprises finerenone in an amount of 10 to 40 mg and dapagliflozin in an amount of 0.5 to 20 mg.

In one embodiment the combination according to the invention comprises finerenone in an amount of 5 to 80 mg and dapagliflozin in an amount of 10 mg. In one embodiment the combination according to the invention comprises finerenone in an amount of 10 to 40 mg and dapagliflozin in an amount of 10 mg.

In one embodiment the combination according to the invention comprises finerenone in an amount of 5 mg and dapagliflozin in an amount of 10 mg. In one embodiment the combination according to the invention comprises finerenone in an amount of 10 mg and dapagliflozin in an amount of 10 mg. In one embodiment the combination according to the invention comprises finerenone in an amount of 20 mg and dapagliflozin in an amount of 10 mg. In one embodiment the combination according to the invention comprises finerenone in an amount of 30 mg and dapagliflozin in an amount of 10 mg. In one embodiment the combination according to the invention comprises finerenone in an amount of 40 mg and dapagliflozin in an amount of 10 mg. In one embodiment the combination according to the invention comprises finerenone in an amount of 50 mg and dapagliflozin in an amount of 10 mg. In one embodiment the combination according to the invention comprises finerenone in an amount of 60 mg and dapagliflozin in an amount of 10 mg. In one embodiment the combination according to the invention comprises finerenone in an amount of 70 mg and dapagliflozin in an amount of 10 mg. In one embodiment the combination according to the invention comprises finerenone in an amount of 80 mg and dapagliflozin in an amount of 10 mg.

In one embodiment the combination according to the invention comprises finerenone and empagliflozin in an amount of 0.5 to 30 mg. In one embodiment the combination according to the invention comprises finerenone and empagliflozin in an amount of 3 to 25 mg. In one embodiment the combination according to the invention comprises finerenone and empagliflozin in an amount of 5 to 25 mg. In one embodiment the combination according to the invention comprises finerenone and empagliflozin in an amount of 5 mg. In one embodiment the combination according to the invention comprises finerenone and empagliflozin in an amount of 10 mg. In one embodiment the combination according to the invention comprises finerenone and empagliflozin in an amount of 15 mg. In one embodiment the combination according to the invention comprises finerenone and empagliflozin in an amount of 20 mg. In one embodiment the combination according to the invention comprises finerenone and empagliflozin in an amount of 25 mg. In one embodiment the combination according to the invention comprises finerenone and empagliflozin in an amount of 30 mg.

In one embodiment the combination according to the invention comprises finerenone and empagliflozin in an amount of 0.5, 1, 2, 3, 4, 5, 6, 7, 8, 9, 10, 15, 20, 25 or 30 mg.

In one embodiment the combination according to the invention comprises finerenone in an amount of 5, 10, 15, 20, 25, 30, 35, 40, 45, 50, 55, 60, 65, 70, 75 or 80 mg and empagliflozin.

In one embodiment the combination according to the invention comprises finerenone in an amount of 5 to 80 mg and empagliflozin in an amount of 0.5 to 30 mg. In one embodiment the combination according to the invention comprises finerenone in an amount of 10 to 40 mg and empagliflozin in an amount of 0.5 to 30 mg.

In one embodiment the combination according to the invention comprises finerenone in an amount of 5 to 80 mg and empagliflozin in an amount of 5 mg. In one embodiment the combination according to the invention comprises finerenone in an amount of 10 to 40 mg and empagliflozin in an amount of 5 mg.

In one embodiment the combination according to the invention comprises finerenone in an amount of 5 to 80 mg and empagliflozin in an amount of 10 mg. In one embodiment the combination according to the invention comprises finerenone in an amount of 10 to 40 mg and empagliflozin in an amount of 10 mg.

In one embodiment the combination according to the invention comprises finerenone in an amount of 5 to 80 mg and empagliflozin in an amount of 15 mg. In one embodiment the combination according to the invention comprises finerenone in an amount of 10 to 40 mg and empagliflozin in an amount of 15 mg.

In one embodiment the combination according to the invention comprises finerenone in an amount of 5 to 80 mg and empagliflozin in an amount of 20 mg. In one embodiment the combination according to the invention comprises finerenone in an amount of 10 to 40 mg and empagliflozin in an amount of 20 mg.

In one embodiment the combination according to the invention comprises finerenone in an amount of 5 to 80 mg and empagliflozin in an amount of 25 mg. In one embodiment the combination according to the invention comprises finerenone in an amount of 10 to 40 mg and empagliflozin in an amount of 25 mg.

In one embodiment the combination according to the invention comprises finerenone in an amount of 5 to 80 mg and empagliflozin in an amount of 30 mg. In one embodiment the combination according to the invention comprises finerenone in an amount of 10 to 40 mg and empagliflozin in an amount of 30 mg.

In one embodiment the combination according to the invention comprises finerenone in an amount of 5 mg and empagliflozin in an amount of 5 mg. In one embodiment the combination according to the invention comprises finerenone in an amount of 10 mg and empagliflozin in an amount of 5 mg. In one embodiment the combination according to the invention comprises finerenone in an amount of 20 mg and empagliflozin in an amount of 5 mg. In one embodiment the combination according to the invention comprises finerenone in an amount of 30 mg and empagliflozin in an amount of 5 mg. In one embodiment the combination according to the invention comprises finerenone in an amount of 40 mg and empagliflozin in an amount of 5 mg. In one embodiment the combination according to the invention comprises finerenone in an amount of 50 mg and empagliflozin in an amount of 5 mg. In one embodiment the combination according to the invention comprises finerenone in an amount of 60 mg and empagliflozin in an amount of 5 mg. In one embodiment the combination according to the invention comprises finerenone in an amount of 70 mg and empagliflozin in an amount of 5 mg. In one embodiment the combination according to the invention comprises finerenone in an amount of 80 mg and empagliflozin in an amount of 5 mg.

In one embodiment the combination according to the invention comprises finerenone in an amount of 5 mg and empagliflozin in an amount of 10 mg. In one embodiment the combination according to the invention comprises finerenone in an amount of 10 mg and empagliflozin in an amount of 10 mg. In one embodiment the combination according to the invention comprises finerenone in an amount of 20 mg and empagliflozin in an amount of 10 mg. In one embodiment the combination according to the invention comprises finerenone in an amount of 30 mg and empagliflozin in an amount of 10 mg. In one embodiment the combination according to the invention comprises finerenone in an amount of 40 mg and empagliflozin in an amount of 10 mg. In one embodiment the combination according to the invention comprises finerenone in an amount of 50 mg and empagliflozin in an amount of 10 mg. In one embodiment the combination according to the invention comprises finerenone in an amount of 60 mg and empagliflozin in an amount of 10 mg. In one embodiment the combination according to the invention comprises finerenone in an amount of 70 mg and empagliflozin in an amount of 10 mg. In one embodiment the combination according to the invention comprises finerenone in an amount of 80 mg and empagliflozin in an amount of 10 mg.

In one embodiment the combination according to the invention comprises finerenone in an amount of 5 mg and empagliflozin in an amount of 15 mg. In one embodiment the combination according to the invention comprises finerenone in an amount of 10 mg and empagliflozin in an amount of 15 mg. In one embodiment the combination according to the invention comprises finerenone in an amount of 20 mg and empagliflozin in an amount of 15 mg. In one embodiment the combination according to the invention comprises finerenone in an amount of 30 mg and empagliflozin in an amount of 15 mg. In one embodiment the combination according to the invention comprises finerenone in an amount of 40 mg and empagliflozin in an amount of 15 mg. In one embodiment the combination according to the invention comprises finerenone in an amount of 50 mg and empagliflozin in an amount of 15 mg. In one embodiment the combination according to the invention comprises finerenone in an amount of 60 mg and empagliflozin in an amount of 15 mg. In one embodiment the combination according to the invention comprises finerenone in an amount of 70 mg and empagliflozin in an amount of 15 mg. In one embodiment the combination according to the invention comprises finerenone in an amount of 80 mg and empagliflozin in an amount of 15 mg.

In one embodiment the combination according to the invention comprises finerenone in an amount of 5 mg and empagliflozin in an amount of 20 mg. In one embodiment the combination according to the invention comprises finerenone in an amount of 10 mg and empagliflozin in an amount of 20 mg. In one embodiment the combination according to the invention comprises finerenone in an amount of 20 mg and empagliflozin in an amount of 20 mg. In one embodiment the combination according to the invention comprises finerenone in an amount of 30 mg and empagliflozin in an amount of 20 mg. In one embodiment the combination according to the invention comprises finerenone in an amount of 40 mg and empagliflozin in an amount of 20 mg. In one embodiment the combination according to the invention comprises finerenone in an amount of 50 mg and empagliflozin in an amount of 20 mg. In one embodiment the combination according to the invention comprises finerenone in an amount of 60 mg and empagliflozin in an amount of 20 mg. In one embodiment the combination according to the invention comprises finerenone in an amount of 70 mg and empagliflozin in an amount of 20 mg. In one embodiment the combination according to the invention comprises finerenone in an amount of 80 mg and empagliflozin in an amount of 20 mg.

In one embodiment the combination according to the invention comprises finerenone in an amount of 5 mg and empagliflozin in an amount of 25 mg. In one embodiment the combination according to the invention comprises finerenone in an amount of 10 mg and empagliflozin in an amount of 25 mg. In one embodiment the combination according to the invention comprises finerenone in an amount of 20 mg and empagliflozin in an amount of 25 mg. In one embodiment the combination according to the invention comprises finerenone in an amount of 30 mg and empagliflozin in an amount of 25 mg. In one embodiment the combination according to the invention comprises finerenone in an amount of 40 mg and empagliflozin in an amount of 25 mg. In one embodiment the combination according to the invention comprises finerenone in an amount of 50 mg and empagliflozin in an amount of 25 mg. In one embodiment the combination according to the invention comprises finerenone in an amount of 60 mg and empagliflozin in an amount of 25 mg. In one embodiment the combination according to the invention comprises finerenone in an amount of 70 mg and empagliflozin in an amount of 25 mg. In one embodiment the combination according to the invention comprises finerenone in an amount of 80 mg and empagliflozin in an amount of 25 mg.

In one embodiment the combination according to the invention comprises finerenone in an amount of 5 mg and empagliflozin in an amount of 30 mg. In one embodiment the combination according to the invention comprises finerenone in an amount of 10 mg and empagliflozin in an amount of 30 mg. In one embodiment the combination according to the invention comprises finerenone in an amount of 20 mg and empagliflozin in an amount of 30 mg. In one embodiment the combination according to the invention comprises finerenone in an amount of 30 mg and empagliflozin in an amount of 30 mg. In one embodiment the combination according to the invention comprises finerenone in an amount of 40 mg and empagliflozin in an amount of 30 mg. In one embodiment the combination according to the invention comprises finerenone in an amount of 50 mg and empagliflozin in an amount of 30 mg. In one embodiment the combination according to the invention comprises finerenone in an amount of 60 mg and empagliflozin in an amount of 30 mg. In one embodiment the combination according to the invention comprises finerenone in an amount of 70 mg and empagliflozin in an amount of 30 mg. In one embodiment the combination according to the invention comprises finerenone in an amount of 80 mg and empagliflozin in an amount of 30 mg.

In one embodiment the combination according to the invention comprises finerenone in an amount of 5 to 80 mg and empagliflozin in an amount of 0.5 to 30 mg. In one embodiment the combination according to the invention comprises finerenone in an amount of 10 to 40 mg and empagliflozin in an amount of 0.5 to 30 mg.

In one embodiment the combination according to the invention comprises finerenone in an amount of 5 to 80 mg and empagliflozin in an amount of 5 mg. In one embodiment the combination according to the invention comprises finerenone in an amount of 10 to 40 mg and empagliflozin in an amount of 5 mg.

In one embodiment the combination according to the invention comprises finerenone in an amount of 5 to 80 mg and empagliflozin in an amount of 10 mg. In one embodiment the combination according to the invention comprises finerenone in an amount of 10 to 40 mg and empagliflozin in an amount of 10 mg.

In one embodiment the combination according to the invention comprises finerenone in an amount of 5 to 80 mg and empagliflozin in an amount of 15 mg. In one embodiment the combination according to the invention comprises finerenone in an amount of 10 to 40 mg and empagliflozin in an amount of 15 mg.

In one embodiment the combination according to the invention comprises finerenone in an amount of 5 to 80 mg and empagliflozin in an amount of 20 mg. In one embodiment the combination according to the invention comprises finerenone in an amount of 10 to 40 mg and empagliflozin in an amount of 20 mg.

In one embodiment the combination according to the invention comprises finerenone in an amount of 5 to 80 mg and empagliflozin in an amount of 25 mg. In one embodiment the combination according to the invention comprises finerenone in an amount of 10 to 40 mg and empagliflozin in an amount of 25 mg.

In one embodiment the combination according to the invention comprises finerenone in an amount of 5 to 80 mg and empagliflozin in an amount of 30 mg. In one embodiment the combination according to the invention comprises finerenone in an amount of 10 to 40 mg and empagliflozin in an amount of 30 mg.

In one embodiment the combination according to the invention comprises finerenone in an amount of 5 mg and empagliflozin in an amount of 5 mg. In one embodiment the combination according to the invention comprises finerenone in an amount of 10 mg and empagliflozin in an amount of 5 mg. In one embodiment the combination according to the invention comprises finerenone in an amount of 20 mg and empagliflozin in an amount of 5 mg. In one embodiment the combination according to the invention comprises finerenone in an amount of 30 mg and empagliflozin in an amount of 5 mg. In one embodiment the combination according to the invention comprises finerenone in an amount of 40 mg and empagliflozin in an amount of 5 mg. In one embodiment the combination according to the invention comprises finerenone in an amount of 50 mg and empagliflozin in an amount of 5 mg. In one embodiment the combination according to the invention comprises finerenone in an amount of 60 mg and empagliflozin in an amount of 5 mg. In one embodiment the combination according to the invention comprises finerenone in an amount of 70 mg and empagliflozin in an amount of 5 mg. In one embodiment the combination according to the invention comprises finerenone in an amount of 80 mg and empagliflozin in an amount of 5 mg.

In one embodiment the combination according to the invention comprises finerenone in an amount of 5 mg and empagliflozin in an amount of 10 mg. In one embodiment the combination according to the invention comprises finerenone in an amount of 10 mg and empagliflozin in an amount of 10 mg. In one embodiment the combination according to the invention comprises finerenone in an amount of 20 mg and empagliflozin in an amount of 10 mg. In one embodiment the combination according to the invention comprises finerenone in an amount of 30 mg and empagliflozin in an amount of 10 mg. In one embodiment the combination according to the invention comprises finerenone in an amount of 40 mg and empagliflozin in an amount of 10 mg. In one embodiment the combination according to the invention comprises finerenone in an amount of 50 mg and empagliflozin in an amount of 10 mg. In one embodiment the combination according to the invention comprises finerenone in an amount of 60 mg and empagliflozin in an amount of 10 mg. In one embodiment the combination according to the invention comprises finerenone in an amount of 70 mg and empagliflozin in an amount of 10 mg. In one embodiment the combination according to the invention comprises finerenone in an amount of 80 mg and empagliflozin in an amount of 10 mg.

In one embodiment the combination according to the invention comprises finerenone in an amount of 5 mg and empagliflozin in an amount of 15 mg. In one embodiment the combination according to the invention comprises finerenone in an amount of 10 mg and empagliflozin in an amount of 15 mg. In one embodiment the combination according to the invention comprises finerenone in an amount of 20 mg and empagliflozin in an amount of 15 mg. In one embodiment the combination according to the invention comprises finerenone in an amount of 30 mg and empagliflozin in an amount of 15 mg. In one embodiment the combination according to the invention comprises finerenone in an amount of 40 mg and empagliflozin in an amount of 15 mg. In one embodiment the combination according to the invention comprises finerenone in an amount of 50 mg and empagliflozin in an amount of 15 mg. In one embodiment the combination according to the invention comprises finerenone in an amount of 60 mg and empagliflozin in an amount of 15 mg. In one embodiment the combination according to the invention comprises finerenone in an amount of 70 mg and empagliflozin in an amount of 15 mg. In one embodiment the combination according to the invention comprises finerenone in an amount of 80 mg and empagliflozin in an amount of 15 mg.

In one embodiment the combination according to the invention comprises finerenone in an amount of 5 mg and empagliflozin in an amount of 20 mg. In one embodiment the combination according to the invention comprises finerenone in an amount of 10 mg and empagliflozin in an amount of 20 mg. In one embodiment the combination according to the invention comprises finerenone in an amount of 20 mg and empagliflozin in an amount of 20 mg. In one embodiment the combination according to the invention comprises finerenone in an amount of 30 mg and empagliflozin in an amount of 20 mg. In one embodiment the combination according to the invention comprises finerenone in an amount of 40 mg and empagliflozin in an amount of 20 mg. In one embodiment the combination according to the invention comprises finerenone in an amount of 50 mg and empagliflozin in an amount of 20 mg. In one embodiment the combination according to the invention comprises finerenone in an amount of 60 mg and empagliflozin in an amount of 20 mg. In one embodiment the combination according to the invention comprises finerenone in an amount of 70 mg and empagliflozin in an amount of 20 mg. In one embodiment the combination according to the invention comprises finerenone in an amount of 80 mg and empagliflozin in an amount of 20 mg.

In one embodiment the combination according to the invention comprises finerenone in an amount of 5 mg and empagliflozin in an amount of 25 mg. In one embodiment the combination according to the invention comprises finerenone in an amount of 10 mg and empagliflozin in an amount of 25 mg. In one embodiment the combination according to the invention comprises finerenone in an amount of 20 mg and empagliflozin in an amount of 25 mg. In one embodiment the combination according to the invention comprises finerenone in an amount of 30 mg and empagliflozin in an amount of 25 mg. In one embodiment the combination according to the invention comprises finerenone in an amount of 40 mg and empagliflozin in an amount of 25 mg. In one embodiment the combination according to the invention comprises finerenone in an amount of 50 mg and empagliflozin in an amount of 25 mg. In one embodiment the combination according to the invention comprises finerenone in an amount of 60 mg and empagliflozin in an amount of 25 mg. In one embodiment the combination according to the invention comprises finerenone in an amount of 70 mg and empagliflozin in an amount of 25 mg. In one embodiment the combination according to the invention comprises finerenone in an amount of 80 mg and empagliflozin in an amount of 25 mg.

In one embodiment the combination according to the invention comprises finerenone in an amount of 5 mg and empagliflozin in an amount of 30 mg. In one embodiment the combination according to the invention comprises finerenone in an amount of 10 mg and empagliflozin in an amount of 30 mg. In one embodiment the combination according to the invention comprises finerenone in an amount of 20 mg and empagliflozin in an amount of 30 mg. In one embodiment the combination according to the invention comprises finerenone in an amount of 30 mg and empagliflozin in an amount of 30 mg. In one embodiment the combination according to the invention comprises finerenone in an amount of 40 mg and empagliflozin in an amount of 30 mg. In one embodiment the combination according to the invention comprises finerenone in an amount of 50 mg and empagliflozin in an amount of 30 mg. In one embodiment the combination according to the invention comprises finerenone in an amount of 60 mg and empagliflozin in an amount of 30 mg. In one embodiment the combination according to the invention comprises finerenone in an amount of 70 mg and empagliflozin in an amount of 30 mg. In one embodiment the combination according to the invention comprises finerenone in an amount of 80 mg and empagliflozin in an amount of 30 mg.

In one embodiment the combination according to the invention comprises finerenone and ertugliflozin in an amount of 0.5 to 20 mg. In one embodiment the combination according to the invention comprises finerenone and ertugliflozin in an amount of 3 to 15 mg. In one embodiment the combination according to the invention comprises finerenone and ertugliflozin in an amount of 5 to 15 mg. In one embodiment the combination according to the invention comprises finerenone and ertugliflozin in an amount of 5 mg. In one embodiment the combination according to the invention comprises finerenone and ertugliflozin in an amount of 10 mg. In one embodiment the combination according to the invention comprises finerenone and ertugliflozin in an amount of 15 mg. In one embodiment the combination according to the invention comprises finerenone and ertugliflozin in an amount of 20 mg.

In one embodiment the combination according to the invention comprises finerenone and ertugliflozin in an amount of 0.5, 1, 2, 3, 4, 5, 6, 7, 8, 9, 10, 11, 12, 13, 14, 15, 16, 17, 18, 19 or 20 mg.

In one embodiment the combination according to the invention comprises finerenone in an amount of 5, 10, 15, 20, 25, 30, 35, 40, 45, 50, 55, 60, 65, 70, 75 or 80 mg and ertugliflozin.

In one embodiment the combination according to the invention comprises finerenone in an amount of 5 to 80 mg and ertugliflozin in an amount of 0.5 to 20 mg. In one embodiment the combination according to the invention comprises finerenone in an amount of 10 to 40 mg and ertugliflozin in an amount of 0.5 to 20 mg.

In one embodiment the combination according to the invention comprises finerenone in an amount of 5 to 80 mg and ertugliflozin in an amount of 5 mg. In one embodiment the combination according to the invention comprises finerenone in an amount of 10 to 40 mg and ertugliflozin in an amount of 5 mg.

In one embodiment the combination according to the invention comprises finerenone in an amount of 5 to 80 mg and ertugliflozin in an amount of 10 mg. In one embodiment the combination according to the invention comprises finerenone in an amount of 10 to 40 mg and ertugliflozin in an amount of 10 mg.

In one embodiment the combination according to the invention comprises finerenone in an amount of 5 to 80 mg and ertugliflozin in an amount of 15 mg. In one embodiment the combination according to the invention comprises finerenone in an amount of 10 to 40 mg and ertugliflozin in an amount of 15 mg.

In one embodiment the combination according to the invention comprises finerenone in an amount of 5 to 80 mg and ertugliflozin in an amount of 20 mg. In one embodiment the combination according to the invention comprises finerenone in an amount of 10 to 40 mg and ertugliflozin in an amount of 20 mg.

In one embodiment the combination according to the invention comprises finerenone in an amount of 5 mg and ertugliflozin in an amount of 5 mg. In one embodiment the combination according to the invention comprises finerenone in an amount of 10 mg and ertugliflozin in an amount of 5 mg. In one embodiment the combination according to the invention comprises finerenone in an amount of 20 mg and ertugliflozin in an amount of 5 mg. In one embodiment the combination according to the invention comprises finerenone in an amount of 30 mg and ertugliflozin in an amount of 5 mg. In one embodiment the combination according to the invention comprises finerenone in an amount of 40 mg and ertugliflozin in an amount of 5 mg. In one embodiment the combination according to the invention comprises finerenone in an amount of 50 mg and ertugliflozin in an amount of 5 mg. In one embodiment the combination according to the invention comprises finerenone in an amount of 60 mg and ertugliflozin in an amount of 5 mg. In one embodiment the combination according to the invention comprises finerenone in an amount of 70 mg and ertugliflozin in an amount of 5 mg. In one embodiment the combination according to the invention comprises finerenone in an amount of 80 mg and ertugliflozin in an amount of 5 mg.

In one embodiment the combination according to the invention comprises finerenone in an amount of 5 mg and ertugliflozin in an amount of 10 mg. In one embodiment the combination according to the invention comprises finerenone in an amount of 10 mg and ertugliflozin in an amount of 10 mg. In one embodiment the combination according to the invention comprises finerenone in an amount of 20 mg and ertugliflozin in an amount of 10 mg. In one embodiment the combination according to the invention comprises finerenone in an amount of 30 mg and ertugliflozin in an amount of 10 mg. In one embodiment the combination according to the invention comprises finerenone in an amount of 40 mg and ertugliflozin in an amount of 10 mg. In one embodiment the combination according to the invention comprises finerenone in an amount of 50 mg and ertugliflozin in an amount of 10 mg. In one embodiment the combination according to the invention comprises finerenone in an amount of 60 mg and ertugliflozin in an amount of 10 mg. In one embodiment the combination according to the invention comprises finerenone in an amount of 70 mg and ertugliflozin in an amount of 10 mg. In one embodiment the combination according to the invention comprises finerenone in an amount of 80 mg and ertugliflozin in an amount of 10 mg.

In one embodiment the combination according to the invention comprises finerenone in an amount of 5 mg and ertugliflozin in an amount of 15 mg. In one embodiment the combination according to the invention comprises finerenone in an amount of 10 mg and ertugliflozin in an amount of 15 mg. In one embodiment the combination according to the invention comprises finerenone in an amount of 20 mg and ertugliflozin in an amount of 15 mg. In one embodiment the combination according to the invention comprises finerenone in an amount of 30 mg and ertugliflozin in an amount of 15 mg. In one embodiment the combination according to the invention comprises finerenone in an amount of 40 mg and ertugliflozin in an amount of 15 mg. In one embodiment the combination according to the invention comprises finerenone in an amount of 50 mg and ertugliflozin in an amount of 15 mg. In one embodiment the combination according to the invention comprises finerenone in an amount of 60 mg and ertugliflozin in an amount of 15 mg. In one embodiment the combination according to the invention comprises finerenone in an amount of 70 mg and ertugliflozin in an amount of 15 mg. In one embodiment the combination according to the invention comprises finerenone in an amount of 80 mg and ertugliflozin in an amount of 15 mg.

In one embodiment the combination according to the invention comprises finerenone in an amount of 5 mg and ertugliflozin in an amount of 20 mg. In one embodiment the combination according to the invention comprises finerenone in an amount of 10 mg and ertugliflozin in an amount of 20 mg. In one embodiment the combination according to the invention comprises finerenone in an amount of 20 mg and ertugliflozin in an amount of 20 mg. In one embodiment the combination according to the invention comprises finerenone in an amount of 30 mg and ertugliflozin in an amount of 20 mg. In one embodiment the combination according to the invention comprises finerenone in an amount of 40 mg and ertugliflozin in an amount of 20 mg. In one embodiment the combination according to the invention comprises finerenone in an amount of 50 mg and ertugliflozin in an amount of 20 mg. In one embodiment the combination according to the invention comprises finerenone in an amount of 60 mg and ertugliflozin in an amount of 20 mg. In one embodiment the combination according to the invention comprises finerenone in an amount of 70 mg and ertugliflozin in an amount of 20 mg. In one embodiment the combination according to the invention comprises finerenone in an amount of 80 mg and ertugliflozin in an amount of 20 mg.

In one embodiment the combination according to the invention comprises finerenone in an amount of 5 to 80 mg and ertugliflozin in an amount of 0.5 to 20 mg. In one embodiment the combination according to the invention comprises finerenone in an amount of 10 to 40 mg and ertugliflozin in an amount of 0.5 to 20 mg.

In one embodiment the combination according to the invention comprises finerenone in an amount of 5 to 80 mg and ertugliflozin in an amount of 5 mg. In one embodiment the combination according to the invention comprises finerenone in an amount of 10 to 40 mg and ertugliflozin in an amount of 5 mg.

In one embodiment the combination according to the invention comprises finerenone in an amount of 5 to 80 mg and ertugliflozin in an amount of 10 mg. In one embodiment the combination according to the invention comprises finerenone in an amount of 10 to 40 mg and ertugliflozin in an amount of 10 mg.

In one embodiment the combination according to the invention comprises finerenone in an amount of 5 to 80 mg and ertugliflozin in an amount of 15 mg. In one embodiment the combination according to the invention comprises finerenone in an amount of 10 to 40 mg and ertugliflozin in an amount of 15 mg.

In one embodiment the combination according to the invention comprises finerenone in an amount of 5 to 80 mg and ertugliflozin in an amount of 20 mg. In one embodiment the combination according to the invention comprises finerenone in an amount of 10 to 40 mg and ertugliflozin in an amount of 20 mg.

In one embodiment the combination according to the invention comprises finerenone in an amount of 5 mg and ertugliflozin in an amount of 5 mg. In one embodiment the combination according to the invention comprises finerenone in an amount of 10 mg and ertugliflozin in an amount of 5 mg. In one embodiment the combination according to the invention comprises finerenone in an amount of 20 mg and ertugliflozin in an amount of 5 mg. In one embodiment the combination according to the invention comprises finerenone in an amount of 30 mg and ertugliflozin in an amount of 5 mg. In one embodiment the combination according to the invention comprises finerenone in an amount of 40 mg and ertugliflozin in an amount of 5 mg. In one embodiment the combination according to the invention comprises finerenone in an amount of 50 mg and ertugliflozin in an amount of 5 mg. In one embodiment the combination according to the invention comprises finerenone in an amount of 60 mg and ertugliflozin in an amount of 5 mg. In one embodiment the combination according to the invention comprises finerenone in an amount of 70 mg and ertugliflozin in an amount of 5 mg. In one embodiment the combination according to the invention comprises finerenone in an amount of 80 mg and ertugliflozin in an amount of 5 mg.

In one embodiment the combination according to the invention comprises finerenone in an amount of 5 mg and ertugliflozin in an amount of 10 mg. In one embodiment the combination according to the invention comprises finerenone in an amount of 10 mg and ertugliflozin in an amount of 10 mg. In one embodiment the combination according to the invention comprises finerenone in an amount of 20 mg and ertugliflozin in an amount of 10 mg. In one embodiment the combination according to the invention comprises finerenone in an amount of 30 mg and ertugliflozin in an amount of 10 mg. In one embodiment the combination according to the invention comprises finerenone in an amount of 40 mg and ertugliflozin in an amount of 10 mg. In one embodiment the combination according to the invention comprises finerenone in an amount of 50 mg and ertugliflozin in an amount of 10 mg. In one embodiment the combination according to the invention comprises finerenone in an amount of 60 mg and ertugliflozin in an amount of 10 mg. In one embodiment the combination according to the invention comprises finerenone in an amount of 70 mg and ertugliflozin in an amount of 10 mg. In one embodiment the combination according to the invention comprises finerenone in an amount of 80 mg and ertugliflozin in an amount of 10 mg.

In one embodiment the combination according to the invention comprises finerenone in an amount of 5 mg and ertugliflozin in an amount of 15 mg. In one embodiment the combination according to the invention comprises finerenone in an amount of 10 mg and ertugliflozin in an amount of 15 mg. In one embodiment the combination according to the invention comprises finerenone in an amount of 20 mg and ertugliflozin in an amount of 15 mg. In one embodiment the combination according to the invention comprises finerenone in an amount of 30 mg and ertugliflozin in an amount of 15 mg. In one embodiment the combination according to the invention comprises finerenone in an amount of 40 mg and ertugliflozin in an amount of 15 mg. In one embodiment the combination according to the invention comprises finerenone in an amount of 50 mg and ertugliflozin in an amount of 15 mg. In one embodiment the combination according to the invention comprises finerenone in an amount of 60 mg and ertugliflozin in an amount of 15 mg. In one embodiment the combination according to the invention comprises finerenone in an amount of 70 mg and ertugliflozin in an amount of 15 mg. In one embodiment the combination according to the invention comprises finerenone in an amount of 80 mg and ertugliflozin in an amount of 15 mg.

In one embodiment the combination according to the invention comprises finerenone in an amount of 5 mg and ertugliflozin in an amount of 20 mg. In one embodiment the combination according to the invention comprises finerenone in an amount of 10 mg and ertugliflozin in an amount of 20 mg. In one embodiment the combination according to the invention comprises finerenone in an amount of 20 mg and ertugliflozin in an amount of 20 mg. In one embodiment the combination according to the invention comprises finerenone in an amount of 30 mg and ertugliflozin in an amount of 20 mg. In one embodiment the combination according to the invention comprises finerenone in an amount of 40 mg and ertugliflozin in an amount of 20 mg. In one embodiment the combination according to the invention comprises finerenone in an amount of 50 mg and ertugliflozin in an amount of 20 mg. In one embodiment the combination according to the invention comprises finerenone in an amount of 60 mg and ertugliflozin in an amount of 20 mg. In one embodiment the combination according to the invention comprises finerenone in an amount of 70 mg and ertugliflozin in an amount of 20 mg. In one embodiment the combination according to the invention comprises finerenone in an amount of 80 mg and ertugliflozin in an amount of 20 mg.

Nevertheless, it may in some cases be advantageous to deviate from the amounts specified, depending on body weight, individual behaviour towards the active ingredient, type of preparation and time or interval over which the administration is affected. For instance, less than the aforementioned minimum amounts can be sufficient in some cases, while the upper limit specified has to be exceeded in other cases.

In the case of administration of relatively large amounts, it can be advisable to divide these into several individual doses over the day.

The relative ratios of each compound in the combination can also be selected based on their respective mechanisms of action and the disease biology. The relative ratios of each compound can vary widely.

The present invention also relates to a method for using the combination and compositions thereof, to treat cardiovascular disorders and/or of renal and cardiorenal disorders.

The diseases can be characterized by chronic sodium retention, such as chronic heart and kidney failure.

This method comprises administering to a mammal in need thereof, including a human, an amount of the combination, which is effective to treat the disorder.

In one embodiment the combination according to the invention is used in the treatment and/or prevention of heart failure and/or chronic kidney diseases.

The combination according to the invention is suitable for the prophylaxis and/or treatment of various disorders and disease-related states, in particular for the treatment and/or prophylaxis of
cardiovascular disorders such as congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF);
renal and cardiorenal disorders such as chronic kidney disease (CKD), diabetic and hypertensive kidney disease, cardiorenal syndrome, nephrotic syndrome, hepatorenal syndrome, renal hypoperfusion, intradialytic hypotension, obstructive uropathy, glomerulopathies, IgA nephropathy, glomerulonephritis, glomerulosclerosis, tubulointerstitial diseases, nephropathic diseases such as primary and congenital kidney disease, nephritis, Alport syndrome, kidney inflammation, immunological kidney diseases, kidney transplant rejection, immune complex-induced kidney diseases, nephropathy induced by toxic substances, contrast medium-induced nephropathy; minimal change glomerulonephritis (lipoid), focal segmental glomerulosclerosis (FSGS), amyloidosis, renal cysts, hypertensive nephrosclerosis and nephrotic syndrome (which can be characterized diagnostically, for example, by abnormally reduced creatinine and/or water excretion, abnormally increased blood concentrations of urea, nitrogen, creatinine, altered urine osmolarity or urine volume, increased microalbuminuria, macroalbuminuria, lesions of glomeruli and arterioles, tubular dilatation, hyperphosphataemia and/or the need for dialysis), uraemia, anaemia, electrolyte disturbances, disturbances in bone and carbohydrate metabolism, polycystic kidney disease (PCKD) and of the syndrome of inadequate ADH secretion (SIADH);
edema, pulmonary edema, cerebral edema, renal edema and heart failure-related edema;
cirrhosis;
NASH (non-alcoholic steatohepatitis);
arterial hypertension, resistant hypertension, pulmonary hypertension;
cardiovascular disorders such as hypertension, left ventricular dysfunction, hypertrophic cardiomyopathy, diabetic cardiomyopathy, supraventricular arrythmias, ventricular arrythmias, atrial fibrillation, atrial flutter,
cardiovascular disorders such as stable angina pectoris, unstable angina pectoris, myocardial infarction and sequelae thereof, aneurysms, detrimental vascular remodelling, atherosclerosis, atrial fibrillation, stroke;
shock such as cardiogenic shock, septic shock and anaphylactic shock;
hypertensive kidney disease, peripheral arterial disease (PAD) including claudication and including critical limb ischemia, coronary microvascular dysfunction (CMD) including CMD type 1-4, primary and secondary Raynaud's phenomenon, microcirculation disturbances, peripheral and autonomic neuropathies, diabetic microangiopathies, diabetic retinopathy, diabetic limb ulcers, gangrene, CREST syndrome, erythematous disorders, rheumatic diseases, for promoting wound healing, inflammatory diseases, asthmatic diseases, chronic obstructive pulmonary disease (COPD), acute respiratory distress syndrome (ARDS), acute lung injury (ALI), alpha-1-antitrypsin deficiency (AATD), pulmonary fibrosis, pulmonary emphysema (for example smoking-induced pulmonary emphysema) and cystic fibrosis (CF);
lung disorders and cardiopulmonary disorders such as pulmonary hypertension, disorders of the central nervous system;
fibrotic disorders and other disease manifestations (for example end organ damage affecting brain, kidney or heart);
multiple insults such as ischemia-reperfusion injury, radiocontrast administration, cardiopulmonary bypass surgery, shock and sepsis.

The combination according to the invention is also suitable for the prophylaxis and/or treatment of various disorders and disease-related states, in particular for the treatment and/or prophylaxis of
cardiovascular disorders such as congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF);
renal and cardiorenal disorders such as chronic kidney disease (CKD), non-diabetic chronic kidney disease (ndCKD), diabetic kidney disease, hypertensive kidney disease, cardiorenal syndrome, nephrotic syndrome, hepatorenal syndrome, renal hypoperfusion, intradialytic hypotension, obstructive uropathy, glomerulopathies, IgA nephropathy, glomerulonephritis, glomerulosclerosis, tubulointerstitial diseases, nephropathic diseases such as primary and congenital kidney disease, nephritis, Alport syndrome, kidney inflammation, immunological kidney diseases, kidney transplant rejection, immune complex-induced kidney diseases, nephropathy induced by toxic substances, contrast medium-induced nephropathy; minimal change glomerulonephritis (lipoid), focal segmental glomerulosclerosis (FSGS), amyloidosis, renal cysts, hypertensive nephrosclerosis and nephrotic syndrome (which can be characterized diagnostically, for example, by abnormally reduced creatinine and/or water excretion, abnormally increased blood concentrations of urea, nitrogen, potassium and/or creatinine, altered urine osmolarity or urine volume, increased microalbuminuria, macroalbuminuria, lesions of glomeruli and arterioles, tubular dilatation, hyperphosphataemia and/or the need for dialysis), uraemia, anaemia, electrolyte disturbances (for example hyperkalaemia, hyponatraemia, disturbances in bone and carbohydrate metabolism, polycystic kidney disease (PCKD) and of the syndrome of inadequate ADH secretion (SIADH);
edema, pulmonary edema, cerebral edema, renal edema and heart failure-related edema;
cirrhosis;
NASH (non-alcoholic steatohepatitis);
arterial hypertension, resistant hypertension, pulmonary hypertension, essential hypertension;
cardiovascular disorders such as hypertension, left ventricular dysfunction, hypertrophic cardiomyopathy, diabetic cardiomyopathy, supraventricular arrythmias, ventricular arrythmias, atrial fibrillation, atrial flutter,
cardiovascular disorders such as stable angina pectoris, unstable angina pectoris, myocardial infarction and sequelae thereof, aneurysms, detrimental vascular remodelling, atherosclerosis, atrial fibrillation, stroke;
shock such as cardiogenic shock, septic shock and anaphylactic shock;
hypertensive kidney disease, peripheral arterial disease (PAD) including claudication and including critical limb ischemia, coronary microvascular dysfunction (CMD) including CMD type 1-4, primary and secondary Raynaud's phenomenon, microcirculation disturbances, peripheral and autonomic neuropathies, diabetic microangiopathies, diabetic retinopathy, diabetic limb ulcers, gangrene, CREST syndrome, erythematous disorders, rheumatic diseases, for promoting wound healing, inflammatory diseases, asthmatic diseases, chronic obstructive pulmonary disease (COPD), acute respiratory distress syndrome (ARDS), acute lung injury (ALI), alpha-1-antitrypsin deficiency (AATD), pulmonary fibrosis, pulmonary emphysema (for example smoking-induced pulmonary emphysema) and cystic fibrosis (CF);
lung disorders and cardiopulmonary disorders such as pulmonary hypertension, disorders of the central nervous system;
fibrotic disorders and other disease manifestations (for example end organ damage affecting brain, kidney or heart);
Sleep apnea;
Obesity;
Coronary Artery Disease (CAD),
Acute Kidney Injury (AKI),
Chronic kidney disease after Acute Kidney Injury following Major surgery (AKIM)
multiple insults such as ischemia-reperfusion injury, radiocontrast administration, cardiopulmonary bypass surgery, shock and sepsis.

In one embodiment the combination according to the invention is used in the treatment and/or prophylaxis of diseases characterized by sodium retention. These diseases are for example
cardiovascular disorders such as congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF);
renal and cardiorenal disorders such as chronic kidney disease (CKD), diabetic and hypertensive kidney disease, cardiorenal syndrome, nephrotic syndrome;
edema, pulmonary edema, cerebral edema, renal edema and heart failure-related edema;
cirrhosis; and/or
arterial hypertension, resistant hypertension, pulmonary hypertension.

In one embodiment the combination according to the invention is used in the treatment and/or prophylaxis of chronic kidney disease (CKD). The term chronic kidney disease comprises the terms diabetic kidney disease (DKD) or CKD in diabetes (type 2 diabetes (T2D), type 1 diabetes (T1D)) and non-diabetic chronic kidney disease (ndCKD) including hypertensive CKD. In one example of chronic kidney disease, it can be chronic kidney disease in patients with type-1-diabetes. In another example of chronic kidney disease, it can be chronic kidney disease in patients with type-2-diabetes. In one embodiment the combination according to the invention is used in the treatment and/or prophylaxis of chronic kidney disease, wherein the chronic kidney disease is selected from chronic kidney disease in patients with type-1-diabetes and chronic kidney disease in patients with type-2-diabetes. In one embodiment the combination according to the invention is used in the treatment and/or prophylaxis of diabetic kidney disease (DKD). In one embodiment the combination according to the invention is used in the treatment and/or prophylaxis of non-diabetic chronic kidney disease (ndCKD).

In one embodiment the combination according to the invention is used in the treatment and/or prophylaxis of diabetic retinopathy. In one example of diabetic retinopathy, it can be diabetic retinopathy in patients with type-1-diabetes. In another example of diabetic retinopathy, it can be diabetic retinopathy in patients with type-2-diabetes. In one embodiment the combination according to the invention is used in the treatment and/or prophylaxis of diseases selected from diabetic retinopathy in patients with type-1-diabetes and diabetic retinopathy in in patients with type-2-diabetes.

In one embodiment the combination according to the invention is used in the treatment and/or prophylaxis of diseases selected from congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF), chronic kidney disease (CKD), non-diabetic chronic kidney disease (ndCKD), chronic kidney disease in patients with type-1-diabetes, chronic kidney disease in patients with type-2-diabetes, diabetic retinopathy in patients with type-1-diabetes; diabetic retinopathy in in patients with type-2-diabetes.

In one embodiment the combination according to the invention is used in the treatment and/or prophylaxis of diseases selected from worsening chronic heart failure heart failure with preserved ejection fraction (HFpEF), heart failure with mid-range ejection fraction (HFmrEF, heart failure with reduced ejection fraction (HFrEF), chronic kidney disease (CKD), non-diabetic chronic kidney disease (ndCKD), chronic kidney disease in patients with type-1-diabetes, chronic kidney disease in patients with type-2-diabetes, diabetic retinopathy in in patients with type-1-diabetes, diabetic retinopathy in in patients with type-2-diabetes.

In one embodiment the combination according to the invention is used in the treatment and/or prophylaxis of diseases selected from worsening chronic heart failure (WCHF), heart failure with preserved ejection fraction (HFpEF), heart failure with mid-range ejection fraction (HFmrEF), heart failure with reduced ejection fraction (HFrEF). In one embodiment the combination according to the invention can be used for treatment and/or prophylaxis of the diseases or disorders listed above, wherein the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention is used in the treatment and/or prevention of heart failure (congestive, acute, worsening and chronic, independent of ejection fraction), cardiorenal syndrome, CKD, diabetic and hypertensive kidney disease, nephrotic syndrome, hepatorenal syndrome, edema, cirrhosis, NASH (non-alcoholic steatohepatitis) and/or fibrotic disorders. In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention can be used for the prophylaxis and/or treatment of heart failure (independent of ejection fraction) and/or chronic kidney disease. In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention is used in the treatment and/or prevention of cardiovascular disorders such as congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF). In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention is used in the treatment and/or prevention of renal and cardiorenal disorders such as chronic kidney disease (CKD), diabetic and hypertensive kidney disease, cardiorenal syndrome, nephrotic syndrome, hepatorenal syndrome, renal hypoperfusion, intradialytic hypotension, obstructive uropathy, glomerulopathies, IgA nephropathy, glomerulonephritis, glomerulosclerosis, tubulointerstitial diseases, nephropathic diseases such as primary and congenital kidney disease, nephritis, Alport syndrome, kidney inflammation, immunological kidney diseases, kidney transplant rejection, immune complex-induced kidney diseases, nephropathy induced by toxic substances, contrast medium-induced nephropathy; minimal change glomerulonephritis (lipoid), focal segmental glomerulosclerosis (FSGS), amyloidosis, renal cysts, hypertensive nephrosclerosis and nephrotic syndrome (which can be characterized diagnostically, for example, by abnormally reduced creatinine and/or water excretion, abnormally increased blood concentrations of urea, nitrogen, potassium and/or creatinine, altered urine osmolarity or urine volume, increased microalbuminuria, macroalbuminuria, lesions of glomeruli and arterioles, tubular dilatation, hyperphosphataemia and/or the need for dialysis), uraemia, anaemia, electrolyte disturbances (for example hyperkalaemia, hyponatraemia, disturbances in bone and carbohydrate metabolism, polycystic kidney disease (PCKD) and/or of the syndrome of inadequate ADH secretion (SIADH). In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention is used in the treatment and/or prevention of edema, pulmonary edema, cerebral edema, renal edema and/or heart failure-related edema. In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention is used in the treatment and/or prevention of cirrhosis and/or NASH (non-alcoholic steatohepatitis). In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention is used in the treatment and/or prevention of essential hypertension, arterial hypertension, resistant hypertension and/or pulmonary hypertension. In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination according to the invention is used in the treatment and/or prevention of arterial hypertension, resistant hypertension and/or pulmonary hypertension. In this embodiment the SGLT2 inhibitor can be selected from the group of consisting of canagliflozin, dapagliflozin, empagliflozin, ertugliflozin, ipragliflozin, remogliflozin, sergliflozin, sotagliflozin and tofogliflozin.

In one embodiment the combination comprises finerenone and canagliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment, the combination comprises finerenone in an amount of 5 mg, 10 mg, 20 mg, 30 mg or 40 mg and canagliflozin in an amount of 100 mg or 300 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and canagliflozin in an amount of 100 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and canagliflozin in an amount of 100 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and canagliflozin in an amount of 100 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and canagliflozin in an amount of 100 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and canagliflozin in an amount of 100 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and canagliflozin in an amount of 100 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and canagliflozin in an amount of 300 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and canagliflozin in an amount of 300 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and canagliflozin in an amount of 300 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and canagliflozin in an amount of 300 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and canagliflozin in an amount of 300 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and canagliflozin in an amount of 100 or 300 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and canagliflozin in an amount of 100 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and canagliflozin in an amount of 100 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and canagliflozin in an amount of 100 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and canagliflozin in an amount of 100 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and canagliflozin in an amount of 100 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and canagliflozin in an amount of 100 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and canagliflozin in an amount of 300 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and canagliflozin in an amount of 300 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and canagliflozin in an amount of 300 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and canagliflozin in an amount of 300 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and canagliflozin in an amount of 300 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and canagliflozin in an amount of 100 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and canagliflozin in an amount of 100 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and canagliflozin in an amount of 100 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and canagliflozin in an amount of 100 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and canagliflozin in an amount of 100 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and canagliflozin in an amount of 100 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and canagliflozin in an amount of 300 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and canagliflozin in an amount of 300 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and canagliflozin in an amount of 300 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and canagliflozin in an amount of 300 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and canagliflozin in an amount of 300 mg, wherein the combination is used in the treatment of a disease selected from diabetic retinopathy, diabetic retinopathy in patients with type-1-diabetes and diabetic retinopathy in patients with type-2-diabetes. In one embodiment the combination comprises finerenone and dapagliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment, the combination comprises finerenone in an amount of 5 mg, 10 mg, 20 mg, 30 mg or 40 mg and dapagliflozin in an amount of 5 mg or 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and dapagliflozin in an amount of 5 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and dapagliflozin in an amount of 5 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and dapagliflozin in an amount of 5 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and dapagliflozin in an amount of 5 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and dapagliflozin in an amount of 5 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and dapagliflozin in an amount of 5 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and dapagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and dapagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and dapagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and dapagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and dapagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and dapagliflozin in an amount of 100 or 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and dapagliflozin in an amount of 5 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and dapagliflozin in an amount of 5 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and dapagliflozin in an amount of 5 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and dapagliflozin in an amount of 5 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and dapagliflozin in an amount of 5 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and dapagliflozin in an amount of 5 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and dapagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and dapagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and dapagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and dapagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and dapagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and dapagliflozin in an amount of 5 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and dapagliflozin in an amount of 5 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and dapagliflozin in an amount of 5 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and dapagliflozin in an amount of 5 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and dapagliflozin in an amount of 5 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and dapagliflozin in an amount of 5 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and dapagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and dapagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and dapagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and dapagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and dapagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment the combination comprises finerenone and empagliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment, the combination comprises finerenone in an amount of 5 mg, 10 mg, 20 mg, 30 mg or 40 mg and empagliflozin in an amount of 10 mg or 25 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and empagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and empagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and empagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and empagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and empagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and empagliflozin in an amount of 10 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and empagliflozin in an amount of 25 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and empagliflozin in an amount of 25 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and empagliflozin in an amount of 25 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and empagliflozin in an amount of 25 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and empagliflozin in an amount of 25 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and empagliflozin in an amount of 100 or 25 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and empagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and empagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and empagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and empagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and empagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and empagliflozin in an amount of 10 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and empagliflozin in an amount of 25 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and empagliflozin in an amount of 25 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and empagliflozin in an amount of 25 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and empagliflozin in an amount of 25 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and empagliflozin in an amount of 25 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and empagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and empagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and empagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and empagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and empagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and empagliflozin in an amount of 10 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and empagliflozin in an amount of 25 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and empagliflozin in an amount of 25 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and empagliflozin in an amount of 25 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and empagliflozin in an amount of 25 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and empagliflozin in an amount of 25 mg, wherein the combination is used in the treatment of a disease selected from diabetic retinopathy, diabetic retinopathy in patients with type-1-diabetes and diabetic retinopathy in patients with type-2-diabetes.

In one embodiment the combination comprises finerenone and ertugliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment the combination comprises finerenone and ipragliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment the combination comprises finerenone and remogliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment the combination comprises finerenone and sergliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment the combination comprises finerenone and sotagliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

In one embodiment, the combination comprises finerenone in an amount of 5 mg, 10 mg, 20 mg, 30 mg or 40 mg and sotagliflozin in an amount of 200 mg or 400 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and sotagliflozin in an amount of 200 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and sotagliflozin in an amount of 200 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and sotagliflozin in an amount of 200 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and sotagliflozin in an amount of 200 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and sotagliflozin in an amount of 200 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and sotagliflozin in an amount of 200 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg and sotagliflozin in an amount of 400 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 10 mg and sotagliflozin in an amount of 400 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 20 mg and sotagliflozin in an amount of 400 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 30 mg and sotagliflozin in an amount of 400 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 40 mg and sotagliflozin in an amount of 400 mg, wherein the combination is used in the treatment of a disease selected from chronic kidney disease (CKD), hypertensive kidney disease, diabetic kidney disease (DKD), non-diabetic chronic kidney disease (ndCKD), chronic kidney disease in patients with type-1-diabetes and chronic kidney disease in patients with type-2-diabetes.

In one embodiment, the combination comprises finerenone in an amount of 5 mg, 10 mg, 20 mg, 30 mg or 40 mg and sotagliflozin in an amount of 100 or 400 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and sotagliflozin in an amount of 200 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and sotagliflozin in an amount of 200 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and sotagliflozin in an amount of 200 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and sotagliflozin in an amount of 200 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and sotagliflozin in an amount of 200 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and sotagliflozin in an amount of 200 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg and sotagliflozin in an amount of 400 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 10 mg and sotagliflozin in an amount of 400 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 20 mg and sotagliflozin in an amount of 400 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 30 mg and sotagliflozin in an amount of 400 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 40 mg and sotagliflozin in an amount of 400 mg, wherein the combination is used in the treatment of a disease selected from heart failure with preserved ejection fraction (HFpEF), heart failure with mid-range ejection fraction (HFmrEF) or heart failure with reduced ejection fraction (HFrEF).

In one embodiment, the combination comprises finerenone in an amount of 5 mg, 10 mg, 20 mg, 30 mg or 40 mg and sotagliflozin in an amount of 200 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and sotagliflozin in an amount of 200 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and sotagliflozin in an amount of 200 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and sotagliflozin in an amount of 200 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and sotagliflozin in an amount of 200 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and sotagliflozin in an amount of 200 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 5 mg and sotagliflozin in an amount of 400 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 10 mg and sotagliflozin in an amount of 400 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 20 mg and sotagliflozin in an amount of 400 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 30 mg and sotagliflozin in an amount of 400 mg, wherein the combination is used in the treatment of a disease selected from diabetic retinopathy, diabetic retinopathy in patients with type-1-diabetes and diabetic retinopathy in patients with type-2-diabetes.

In one embodiment, the combination comprises finerenone in an amount of 40 mg and sotagliflozin in an amount of 400 mg, wherein the combination is used in the treatment of a disease selected from diabetic retinopathy, diabetic retinopathy in patients with type-1-diabetes and diabetic retinopathy in patients with type-2-diabetes.

In one embodiment the combination comprises finerenone and tofogliflozin. In one embodiment, this combination can be used in the treatment of hypertensive kidney disease, diabetic kidney disease (DKD), heart failure with preserved ejection fraction (HFpEF), heart failure with mid-range ejection fraction (HFmrEF) or heart failure with reduced ejection fraction (HFrEF). In another embodiment, this combination can be used in the treatment of hypertensive kidney disease and/or diabetic kidney disease (DKD). In a further embodiment, this combination heart failure with preserved ejection fraction (HFpEF), heart failure with mid-range ejection fraction (HFmrEF) or heart failure with reduced ejection fraction (HFrEF). In just another embodiment, this combination can be used in the treatment diseases characterized by sodium retention as defined above.

The present invention further provides the use of the combination of the invention for the preparation of a pharmaceutical compositions for the treatment of the aforesaid diseases. Sometimes herein, the term “disorder” and disease” are used as synonyms.

The present invention further provides for the use of the combinations of the invention for production of a medicament for the treatment and/or prevention of the aforesaid diseases.

In one embodiment the combination according to the invention for production of a medicament can be used in the treatment of the aforesaid diseases.

Based upon standard laboratory techniques known to evaluate compounds useful for the treatment of cardiovascular diseases and/or of renal and/or cardiorenal diseases, by standard tests and by standard pharmacological assays for the determination of treatment of the conditions identified above in mammals, and by comparison of these results with the results of known medicaments that are used to treat these conditions, the effective dosage of the finerenone and/or a SGLT2 inhibitor can readily be determined for treatment of each desired indication. The amount of the active ingredient to be administered in the treatment of one of these conditions can vary widely according to such considerations as the particular compound and dosage form employed, the mode of administration, the period of treatment, the age and sex of the patient treated, and the nature and extent of the condition treated.

### Clauses

The following clauses form part of the disclosure and describe further embodiments according to the invention:
1. A combination comprising finerenone or a hydrate, solvate, pharmaceutically acceptable salt thereof, or a polymorph thereof, and a SGLT2 inhibitor, or a hydrate, solvate, pharmaceutically acceptable salt thereof, or a polymorph thereof.
2. The combination according to clause 1, wherein the combination is a fixed combination.
3. The combination according to any one of the preceding clauses, wherein the combination consists of a single dosage form.
4. The combination according to any one of the preceding clauses, wherein the combination consists of two separate dosage forms.
5. The combination according to clause 1 or 4, wherein the combination comprises the components:
   a. one dosage form comprising finerenone or a hydrate, solvate, pharmaceutically acceptable salt thereof, or a polymorph thereof, and
   b. one dosage form comprising a SGLT2 inhibitor a hydrate, solvate, pharmaceutically acceptable salt thereof, or a polymorph thereof.
6. The combination according to clause 5, wherein the components a. and b. are administered separately, sequentially, simultaneously, concurrently or chronologically staggered.
7. The combination according to any one of the preceding clauses 1 to 6, wherein the combination is a combination pack, is part of a combination pack, a kit-of-parts or non-fixed combination.
8. The combination according to any one of the preceding clauses 1 to 7, wherein the SGLT2 inhibitor is selected from the group consisting of canagliflozin, dapagliflozin, empagliflozin, ertugliflozin, ipragliflozin, remogliflozin, sergliflozin, sotagliflozin and tofogliflozin.
9. The combination according to any one of the preceding clauses 1 to 8, wherein the combination is a dosage form for oral administration.
10. The combination according to any one of the preceding clauses 1 to 9, wherein the dosage form or one of the dosage forms or both of the dosage forms is a tablet.
11. The combination according to any one of the preceding clauses 1 to 9, wherein the dosage form or one of the dosage forms or both of the dosage forms is a capsule.
12. The combination according to any one of the preceding clauses 1 to 10, wherein the dosage form or one of the dosage forms or both of the dosage forms is a granulate.
13. The combination according to any one of the preceding clauses 1 to 12, wherein the combination comprises finerenone or a hydrate, solvate, pharmaceutically acceptable salt thereof, or a polymorph thereof, in an amount of
   5 to 80 mg; or
   0.25 mg to 80 mg; or
   0.25 mg to 40 mg; or
   0.25 mg to 20 mg; or
   0.25 mg to 10 mg; or
   0.25 mg to 5 mg.
14. The combination according to any one of the preceding clauses 1 to 13, wherein the combination comprises finerenone or a hydrate, solvate, pharmaceutically acceptable salt thereof, or a polymorph thereof, in an amount of 5 to 80 mg, in particular in an amount of 5 to 70 mg, 5 to 60 mg, 5 to 50 mg, 5 to 40 mg, 10 to 80 mg, 10 to 70 mg, 10 to 60 mg, 10 to 50 mg or 10 to 40 mg.
15. The combination according to any one of the preceding clauses 1 to 14, wherein the combination comprises finerenone or a hydrate, solvate, pharmaceutically acceptable salt thereof, or a polymorph thereof, in an amount of 5 to 70 mg, 5 to 60 mg, 5 to 50 mg, 5 to 40 mg, 10 to 80 mg, 10 to 70 mg, 10 to 60 mg, 10 to 50 mg or 10 to 40 mg.
16. The combination according to any one of the preceding clauses 1 to 15, wherein the combination comprises finerenone or a hydrate, solvate, pharmaceutically acceptable salt thereof, or a polymorph thereof, in an amount 20 to 40 mg.
17. The combination according to any one of the preceding clauses 1 to 16, wherein the combination comprises finerenone or a hydrate, solvate, pharmaceutically acceptable salt thereof, or a polymorph thereof, in an amount of 40 mg, 30 mg, 20 mg or 10 mg.
18. The combination according to any one of the preceding clauses 1 to 17, wherein the combination comprises finerenone or a hydrate, solvate, pharmaceutically acceptable salt thereof, or a polymorph thereof, in an amount of 40 mg.
19. The combination according to any one of the preceding clauses 1 to 18, wherein the combination comprises finerenone or a hydrate, solvate, pharmaceutically acceptable salt thereof, or a polymorph thereof, in an amount of 30 mg.
20. The combination according to any one of the preceding clauses 1 to 20, wherein the combination comprises finerenone or a hydrate, solvate, pharmaceutically acceptable salt thereof, or a polymorph thereof, in an amount of 20 mg.
21. The combination according to any one of the preceding clauses 1 to 20, wherein the combination comprises finerenone or a hydrate, solvate, pharmaceutically acceptable salt thereof, or a polymorph thereof, in an amount of 15 mg.
22. The combination according to any one of the preceding clauses 1 to 21, wherein the combination comprises finerenone or a hydrate, solvate, pharmaceutically acceptable salt thereof, or a polymorph thereof, in an amount of 10 mg.
23. The combination according to any one of the preceding clauses 1 to 22, wherein the combination comprises a SGLT2 inhibitor a hydrate thereof, solvate thereof, pharmaceutically acceptable salt thereof, a prodrug thereof, or a polymorph thereof, in an amount of 0.5 to 400 mg.
24. The combination according to any one of the preceding clauses 1 to 23, wherein the combination comprises a SGLT2 inhibitor a hydrate thereof, solvate thereof, pharmaceutically acceptable salt thereof, a prodrug thereof, or a polymorph thereof, in an amount of 0.5 to 300 mg, 1 to 300 mg, 2 to 200 mg, 3 to 100 mg, 5 to 100 mg, 5 to 90 mg, 5 to 80 mg, 5 to 70 mg, 5 to 60 mg, 5 to 50 mg, 5 to 40 mg, 5 to 30 mg 10 to 90 mg, 10 to 80 mg, 10 to 70 mg, 10 to 60 mg, 10 to 50 mg or 10 to 40 mg, 10 to 30 mg 15 to 90 mg, 15 to 80 mg, 15 to 70 mg, 15 to 60 mg, 15 to 50 mg, 15 to 40 mg or 15 to 30 mg.
25. The combination according to any one of the preceding clauses 1 to 24, wherein the combination comprises a SGLT2 inhibitor a hydrate thereof, solvate thereof, pharmaceutically acceptable salt thereof, a prodrug thereof, or a polymorph thereof, in an amount ofl, 3, 5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290 or 300 mg.
26. The combination according to any one of the preceding clauses 1 to 25, wherein the combination comprises finerenone or a hydrate, solvate, pharmaceutically acceptable salt thereof, or a polymorph thereof in an amount of 5, 10, 15, 20, 25, 30, 35, 40, 45, 50, 55, 60, 65, 70, 75 or 80 mg and canagliflozin or an anhydrate, a hydrate thereof, solvate thereof, pharmaceutically acceptable salt thereof, a prodrug thereof or a polymorph thereof.
27. The combination according to any one of the preceding clauses 1 to 26, wherein the combination comprises finerenone or a hydrate, solvate, pharmaceutically acceptable salt thereof, or a polymorph thereof in an amount of 5, 10, 15, 20, 25, 30, 35, 40, 45, 50, 55, 60, 65, 70, 75 or 80 mg and dapagliflozin or an anhydrate, a hydrate thereof, solvate thereof, pharmaceutically acceptable salt thereof, a prodrug thereof or a polymorph thereof.
28. The combination according to any one of the preceding clauses 1 to 27, wherein the combination comprises finerenone or a hydrate, solvate, pharmaceutically acceptable salt thereof, or a polymorph thereof in an amount of 5, 10, 15, 20, 25, 30, 35, 40, 45, 50, 55, 60, 65, 70, 75 or 80 mg and empagliflozin or an anhydrate, a hydrate thereof, solvate thereof, pharmaceutically acceptable salt thereof, a prodrug thereof or a polymorph thereof.
29. The combination according to any one of the preceding clauses 1 to 28, wherein the combination comprises finerenone or a hydrate, solvate, pharmaceutically acceptable salt thereof, or a polymorph thereof in an amount of 5, 10, 15, 20, 25, 30, 35, 40, 45, 50, 55, 60, 65, 70, 75 or 80 mg and ertugliflozin or an anhydrate, a hydrate thereof, solvate thereof, pharmaceutically acceptable salt thereof, a prodrug thereof or a polymorph thereof.
30. The combination according to any one of the preceding clauses 1 to 29, wherein the combination comprises finerenone or a hydrate, solvate, pharmaceutically acceptable salt thereof, or a polymorph thereof and canagliflozin or an anhydrate, a hydrate thereof, solvate thereof, pharmaceutically acceptable salt thereof, a prodrug thereof or a polymorph thereof in an amount of 5, 10, 20, 30, 30, 40, 50, 60, 70, 80, 90, 100, 110, 120, 130, 140, 150, 160, 170, 180, 190, 200, 210, 220, 230, 240, 250, 260, 270, 280, 290 or 300 mg.
31. The combination according to any one of the preceding clauses 1 to 30, wherein the combination comprises finerenone or a hydrate, solvate, pharmaceutically acceptable salt thereof, or a polymorph thereof and canagliflozin or an anhydrate, a hydrate thereof, solvate thereof, pharmaceutically acceptable salt thereof, a prodrug thereof or a polymorph thereof in an amount of 50, 100, 150, 200, 250 or 300 mg.
32. The combination according to any one of the preceding clauses 1 to 31, wherein the combination comprises finerenone or a hydrate, solvate, pharmaceutically acceptable salt thereof, or a polymorph thereof and dapagliflozin or an anhydrate, a hydrate thereof, solvate thereof, pharmaceutically acceptable salt thereof, a prodrug thereof or a polymorph thereof in an amount of 0.5, 1, 2, 3, 4, 5, 6, 7, 8, 9 or 10 mg.
33. The combination according to any one of the preceding clauses 1 to 32, wherein the combination comprises finerenone or a hydrate, solvate, pharmaceutically acceptable salt thereof, or a polymorph thereof and dapagliflozin or an anhydrate, a hydrate thereof, solvate thereof, pharmaceutically acceptable salt thereof, a prodrug thereof or a polymorph thereof in an amount of 5 or 10 mg.
34. The combination according to any one of the preceding clauses 1 to 33, wherein the combination comprises finerenone or a hydrate, solvate, pharmaceutically acceptable salt thereof, or a polymorph thereof and empagliflozin or an anhydrate, a hydrate thereof, solvate thereof, pharmaceutically acceptable salt thereof, a prodrug thereof or a polymorph thereof in an amount of 0.5, 1, 2, 3, 4, 5, 6, 7, 8, 9, 10, 15, 20, 25 or 30 mg.
35. The combination according to any one of the preceding clauses 1 to 34, wherein the combination comprises finerenone or a hydrate, solvate, pharmaceutically acceptable salt thereof, or a polymorph thereof and empagliflozin or an anhydrate, a hydrate thereof, solvate thereof, pharmaceutically acceptable salt thereof, a prodrug thereof or a polymorph thereof in an amount of 5, 10, 15, 20, or 25 mg.
36. The combination according to any one of the preceding clauses 1 to 35, wherein the combination comprises finerenone or a hydrate, solvate, pharmaceutically acceptable salt thereof, or a polymorph thereof and ertugliflozin or an anhydrate, a hydrate thereof, solvate thereof, pharmaceutically acceptable salt thereof, a prodrug thereof or a polymorph thereof in an amount of 0.5, 1, 2, 3, 4, 5, 6, 7, 8, 9, 10, 11, 12, 13, 14, 15, 16, 17, 18, 19 or 20 mg.
37. The combination according to any one of the preceding clauses 1 to 36 for once daily application.
38. Medicament comprising the combination according to any one of the preceding clauses 1 to 37.
39. Medicament according clause 28 comprising one or more inert, nontoxic, pharmaceutically suitable excipients.
40. The combination according to any one of clauses 1 to 37 or the medicament according to any one of clauses 38 or 39 for use as medicament.
41. The combination according to clause 40 for use as medicament for treating and/or preventing diseases.
42. The combination according to any one of clauses 1 to 37 or the medicament according to any one of clauses 38 or 39 for use as medicament for treating and/or preventing diseases, wherein the diseases is selected from
   cardiovascular disorders such as congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF);
   renal and cardiorenal disorders such as chronic kidney disease (CKD), diabetic and hypertensive kidney disease, cardiorenal syndrome, nephrotic syndrome, hepatorenal syndrome, renal hypoperfusion, intradialytic hypotension, obstructive uropathy, glomerulopathies, IgA nephropathy, glomerulonephritis, glomerulosclerosis, tubulointerstitial diseases, nephropathic diseases such as primary and congenital kidney disease, nephritis, Alport syndrome, kidney inflammation, immunological kidney diseases, kidney transplant rejection, immune complex-induced kidney diseases, nephropathy induced by toxic substances, contrast medium-induced nephropathy; minimal change glomerulonephritis (lipoid), focal segmental glomerulosclerosis (FSGS), amyloidosis, renal cysts, hypertensive nephrosclerosis and nephrotic syndrome (which can be characterized diagnostically, for example, by abnormally reduced creatinine and/or water excretion, abnormally increased blood concentrations of urea, nitrogen, potassium and/or creatinine, altered urine osmolarity or urine volume, increased microalbuminuria, macroalbuminuria, lesions of glomeruli and arterioles, tubular dilatation, hyperphosphataemia and/or the need for dialysis), uraemia, anaemia, electrolyte disturbances (for example hyperkalaemia, hyponatraemia, disturbances in bone and carbohydrate metabolism, polycystic kidney disease (PCKD) and of the syndrome of inadequate ADH secretion (SIADH);
   edema, pulmonary edema, cerebral edema, renal edema and heart failure-related edema;
   cirrhosis;
   NASH (non-alcoholic steatohepatitis);
   arterial hypertension, resistant hypertension, pulmonary hypertension;
   cardiovascular disorders such as hypertension, left ventricular dysfunction, hypertrophic cardiomyopathy, diabetic cardiomyopathy, supraventricular arrythmias, ventricular arrythmias, atrial fibrillation, atrial flutter,
   cardiovascular disorders such as stable angina pectoris, unstable angina pectoris, myocardial infarction and sequelae thereof, aneurysms, detrimental vascular remodelling, atherosclerosis, atrial fibrillation, stroke;
   shock such as cardiogenic shock, septic shock and anaphylactic shock;
   hypertensive kidney disease, peripheral arterial disease (PAD) including claudication and including critical limb ischemia, coronary microvascular dysfunction (CMD) including CMD type 1-4, primary and secondary Raynaud's phenomenon, microcirculation disturbances, peripheral and autonomic neuropathies, diabetic microangiopathies, diabetic retinopathy, diabetic limb ulcers, gangrene, CREST syndrome, erythematous disorders, rheumatic diseases, for promoting wound healing, inflammatory diseases, asthmatic diseases, chronic obstructive pulmonary disease (COPD), acute respiratory distress syndrome (ARDS), acute lung injury (ALI), alpha-1-antitrypsin deficiency (AATD), pulmonary fibrosis, pulmonary emphysema (for example smoking-induced pulmonary emphysema) and cystic fibrosis (CF);
   lung disorders and cardiopulmonary disorders such as pulmonary hypertension, disorders of the central nervous system;
   fibrotic disorders and other disease manifestations (for example end organ damage affecting brain, kidney or heart);
   multiple insults such as ischemia-reperfusion injury, radiocontrast administration, cardiopulmonary bypass surgery, shock and sepsis.
43. The combination for use according any one of clause 41 or 42, wherein the diseases is selected from heart failure (congestive, acute, worsening and chronic, independent of ejection fraction), cardiorenal syndrome, CKD, diabetic and hypertensive kidney disease, nephrotic syndrome, hepatorenal syndrome, edema, cirrhosis, NASH (non-alcoholic steatohepatitis) and/or fibrotic disorders.
44. The combination for use according any one of clause 41 or 42, wherein the diseases is selected from heart failure (independent of ejection fraction) and/or chronic kidney disease.
45. The combination for use according any one of clause 41 or 42, wherein the diseases is selected from cardiovascular disorders such as congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF).
46. The combination for use according any one of clause 41 or 42, wherein the diseases is selected from renal and cardiorenal disorders such as chronic kidney disease (CKD), diabetic and hypertensive kidney disease, cardiorenal syndrome, nephrotic syndrome, hepatorenal syndrome, renal hypoperfusion, intradialytic hypotension, obstructive uropathy, glomerulopathies, IgA nephropathy, glomerulonephritis, glomerulosclerosis, tubulointerstitial diseases, nephropathic diseases such as primary and congenital kidney disease, nephritis, Alport syndrome, kidney inflammation, immunological kidney diseases, kidney transplant rejection, immune complex-induced kidney diseases, nephropathy induced by toxic substances, contrast medium-induced nephropathy; minimal change glomerulonephritis (lipoid), focal segmental glomerulosclerosis (FSGS), amyloidosis, renal cysts, hypertensive nephrosclerosis and nephrotic syndrome (which can be characterized diagnostically, for example, by abnormally reduced creatinine and/or water excretion, abnormally increased blood concentrations of urea, nitrogen, potassium and/or creatinine, altered urine osmolarity or urine volume, increased microalbuminuria, macroalbuminuria, lesions of glomeruli and arterioles, tubular dilatation, hyperphosphataemia and/or the need for dialysis), uraemia, anaemia, electrolyte disturbances (for example hyperkalaemia, hyponatraemia, disturbances in bone and carbohydrate metabolism, polycystic kidney disease (PCKD) and/or of the syndrome of inadequate ADH secretion (SIADH).
47. The combination for use according any one of clause 41 or 42, wherein the diseases is selected from edema, pulmonary edema, cerebral edema, renal edema and/or heart failure-related edema.
48. The combination for use according any one of clause 41 or 42, wherein the diseases is selected from cirrhosis and/or NASH (non-alcoholic steatohepatitis).
49. The combination for use according any one of clause 41 or 42, wherein the diseases is selected from arterial hypertension, resistant hypertension and/or pulmonary hypertension.
50. The combination for use according any one of clause 41 or 42, wherein the diseases is selected from worsening chronic heart failure (WCHF), heart failure with preserved ejection fraction (HFpEF), heart failure with mid-range ejection fraction (HFmrEF), heart failure with reduced ejection fraction (HFrEF), chronic kidney disease (CKD), non-diabetic kidney disease (ndCKD), chronic kidney disease in patients with type-1-diabetes, chronic kidney disease in patients with type-2-diabetes, diabetic retinopathy in in patients with type-1-diabetes, diabetic retinopathy in in patients with type-2-diabetes.
51. The combination for use according any one of clause 41 or 42 or 50, wherein the diseases is selected from worsening chronic heart failure (WCHF), heart failure with preserved ejection fraction (HFpEF), heart failure with mid-range ejection fraction (HFmrEF), heart failure with reduced ejection fraction (HFrEF).
52. The combination for use according any one of clause 41 or 42 or 50, wherein the diseases is selected from chronic kidney disease (CKD), non-diabetic chronic kidney disease (ndCKD), chronic kidney disease in patients with type-1-diabetes, chronic kidney disease in patients with type-2-diabetes.
53. The combination for use according any one of clause 41 or 42 or 50, wherein the diseases is selected from diabetic retinopathy in patients with type-1-diabetes and diabetic retinopathy in in patients with type-2-diabetes.
54. A method of treatment for preventing and/or treatment of diseases in a subject in need thereof using the combination according to any one of clauses 1 to 37 or medicament according to any one of clauses 38 or 39.
55. The method of treatment according to clause 54, wherein the diseases is selected from the diseases listed in any one of clauses 42 to 49 or to clauses 50 to 53.
56. A method of treating and/or preventing diseases in a subject in need thereof comprising administering effective amounts of a mineralocorticoid receptor antagonist and a SGLT2 inhibitor.
57. A method of treating and/or preventing disease in a subject in need thereof according to clause 56 comprising administering effective amounts of finerenone or a hydrate, solvate, pharmaceutically acceptable salt thereof, or a polymorph thereof and a SGLT2 inhibitor, or a hydrate, solvate, pharmaceutically acceptable salt thereof, or a polymorph thereof
58. Use of the combination according to any one of clauses 1 to 37 or medicament according to any one of clauses 38 or 39 for the production of a medicament for the treatment and/or prophylaxis of diseases.
59. The use according to clause 58, wherein the diseases is selected from the diseases listed in any one of clauses 42 to 49.
60. The combination, method or use according to any one of clauses 1 to 59, wherein the combination comprises finerenone or a hydrate, solvate, pharmaceutically acceptable salt thereof, or a polymorph thereof in an amount of 5 mg, 10 mg, 20 mg or 40 mg and empagliflozin or an anhydrate, a hydrate thereof, solvate thereof, pharmaceutically acceptable salt thereof, a prodrug thereof or a polymorph thereof in an amount of 10 mg or 25 mg.
61. The combination, method or use according to any one of clauses 1 to 59, wherein the combination comprises finerenone or a hydrate, solvate, pharmaceutically acceptable salt thereof, or a polymorph thereof in an amount of 5 mg, 10 mg, 20 mg or 40 mg and dapagliflozin or an anhydrate, a hydrate thereof, solvate thereof, pharmaceutically acceptable salt thereof, a prodrug thereof or a polymorph thereof in an amount of 5 mg or 10 mg.
62. The combination, method or use according to any one of clauses 1 to 59, wherein the combination comprises finerenone or a hydrate, solvate, pharmaceutically acceptable salt thereof, or a polymorph thereof in an amount of 5 mg, 10 mg, 20 mg or 40 mg and canagliflozin or an anhydrate, a hydrate thereof, solvate thereof, pharmaceutically acceptable salt thereof, a prodrug thereof or a polymorph thereof in an amount of 100 mg or 300 mg.
63. The combination, method or use according to any one of clauses 1 to 59, wherein the combination comprises finerenone or a hydrate, solvate, pharmaceutically acceptable salt thereof, or a polymorph thereof in an amount of 5 mg, 10 mg, 20 mg or 40 mg and sotagliflozin or an anhydrate, a hydrate thereof, solvate thereof, pharmaceutically acceptable salt thereof, a prodrug thereof or a polymorph thereof in an amount of 200 mg or 400 mg.
64. The combination, method or use according to any one of clauses 1 to 63, wherein the diseases is selected from chronic kidney disease (CKD), diabetic kidney disease (DKD), non-diabetic chronic kidney disease (ndCKD), chronic kidney disease in patients with type-1-diabetes and chronic kidney disease in patients with type-2-diabetes.
65. The combination, method or use according to any one of clauses 1 to 63, wherein the diseases is selected from diabetic retinopathy, diabetic retinopathy in patients with type-1-diabetes and diabetic retinopathy in patients with type-2-diabetes.
66. The combination, method or use according to any one of clauses 1 to 63, wherein the diseases is selected from worsening chronic heart failure (WCHF), heart failure with preserved ejection fraction (HFpEF), heart failure with mid-range ejection fraction (HFmrEF), heart failure with reduced ejection fraction (HFrEF).
67. The combination according to any one of clauses 1 to 66, wherein the SGLT2 inhibitor is selected from dapagliflozin, empagliflozin and canagliflozin.
68. The use of the combination or the combination for use or the method for the treatment and/or prevention of diseases according to any one of clauses 1 to 66, wherein the SGLT2 inhibitor is selected from dapagliflozin, empagliflozin and canagliflozin.

### DESCRIPTION OF THE FIGURES

FIG. 1a: Influence of the SGLT2 inhibitor empagliflozin (groups B-D), of the MR antagonist finerenone (E), and their combination (groups F-H) on urinary volume of awake Wistar rats with activated renin-angiotensin-aldosterone system, which were examined according to section B below, ‘assessment of the physiological effectiveness’ for 24 hours after oral administration of the substances in metabolic cages. n=6-8 animals/per group. +: p<0.05 vs. combination group, ++: p<0.01 vs. combination group, +++: p<0.005 vs. combination group, ‘ns’ means not significant vs. combination group. S means solvent (group A). 1 mg/kg empagliflozin (group B), 3 mg/kg empagliflozin (group C), 10 mg/kg empagliflozin (group D), 1 mg/kg finerenone (group E), 1 mg/kg empagliflozin+1 mg/kg finerenone combination (group F), 3 mg/kg empagliflozin+1 mg/kg finerenone combination (group G), 10 mg/kg empagliflozin+1 mg/kg finerenone combination (group H).

FIG. 1b: Influence of the SGLT2 inhibitor empagliflozin (groups B-D), of the MR antagonist finerenone (E), and their combination (groups F-H) on the urinary glucose concentration of awake Wistar rats with activated renin-angiotensin-aldosterone system, which were examined according to section B below, ‘assessment of the physiological effectiveness’ for 24 hours after oral administration of the substances in metabolic cages. n=6-8 animals/per group. +: p<0.05 vs. combination group, ++: p<0.01 vs. combination group, +++: p<0.005 vs. combination group, ‘ns’ means not significant vs. combination group. S means solvent (group A). 1 mg/kg empagliflozin (group B), 3 mg/kg empagliflozin (group C), 10 mg/kg empagliflozin (group D), 1 mg/kg finerenone (group E), 1 mg/kg empagliflozin+1 mg/kg finerenone combination (group F), 3 mg/kg empagliflozin+1 mg/kg finerenone combination (group G), 10 mg/kg empagliflozin+1 mg/kg finerenone combination (group H).

FIG. 1c: Influence of the SGLT2 inhibitor empagliflozin (groups B-D), of the MR antagonist finerenone (E), and their combination (groups F-H) on the urinary potassium concentration of awake Wistar rats with activated renin-angiotensin-aldosterone system, which were examined according to section B below, ‘assessment of the physiological effectiveness’ for 24 hours after oral administration of the substances in metabolic cages. n=6-8 animals/per group. +: p<0.05 vs. combination group, ++: p<0.01 vs. combination group, +++: p<0.005 vs. combination group, ‘ns’ means not significant vs. combination group. S means solvent (group A). 1 mg/kg empagliflozin (group B), 3 mg/kg empagliflozin (group C), 10 mg/kg empagliflozin (group D), 1 mg/kg finerenone (group E), 1 mg/kg empagliflozin+1 mg/kg finerenone combination (group F), 3 mg/kg empagliflozin+1 mg/kg finerenone combination (group G), 10 mg/kg empagliflozin+1 mg/kg finerenone combination (group H).

FIG. 1d: Influence of the SGLT2 inhibitor empagliflozin (groups B-D), of the MR antagonist finerenone (E), and their combination (groups F-H) on the urinary sodium concentration of awake Wistar rats with activated renin-angiotensin-aldosterone system, which were examined according to section B below, ‘assessment of the physiological effectiveness’ for 24 hours after oral administration of the substances in metabolic cages. n=6-8 animals/per group. +: p<0.05 vs. combination group, ++: p<0.01 vs. combination group, +++: p<0.005 vs. combination group, ‘ns’ means not significant vs. combination group. S means solvent (group A). 1 mg/kg empagliflozin (group B), 3 mg/kg empagliflozin (group C), 10 mg/kg empagliflozin (group D), 1 mg/kg finerenone (group E), 1 mg/kg empagliflozin+1 mg/kg finerenone combination (group F), 3 mg/kg empagliflozin+1 mg/kg finerenone combination (group G), 10 mg/kg empagliflozin+1 mg/kg finerenone combination (group H).

FIG. 2a: Influence of two dosages of the SGLT2 inhibitor empagliflozin (groups B and C), two dosages of the MR antagonist finerenone (groups D and E), and the combination of the low dosages of empagliflozin (SGLT2 inhibitor) and finerenone (group F), respectively, on urinary volume of awake ZDF rats chronically treated with groups A to F, which were examined according to section B below, ‘assessment of the physiological effectiveness’ for 24 hours in metabolic cages. n=14-16 animals/per group. +: p<0.05 vs. combination group, ++: p<0.01 vs. combination group, +++: p<0.005 vs. combination group, ‘ns’ means not significant vs. combination group. S means solvent (group A). 3 mg/kg empagliflozin (group B), 10 mg/kg empagliflozin (group C), 3 mg/kg finerenone (group D), 10 mg/kg finerenone (group E), 3 mg/kg empagliflozin+3 mg/kg finerenone combination (group F).

FIG. 2b: Influence of two dosages of the SGLT2 inhibitor empagliflozin (groups B and C), two dosages of the MR antagonist finerenone (groups D and E), and the combination of the low dosages of empagliflozin (SGLT2 inhibitor) and finerenone (group F), respectively, on the urinary glucose concentration of awake ZDF rats chronically treated with groups A to F, which were examined according to section B below, ‘assessment of the physiological effectiveness’ for 24 hours in metabolic cages. n=14-16 animals/per group. +: p<0.05 vs. combination group, ++: p<0.01 vs. combination group, +++: p<0.005 vs. combination group, ‘ns’ means not significant vs. combination group. S means solvent (group A). 3 mg/kg empagliflozin (group B), 10 mg/kg empagliflozin (group C), 3 mg/kg finerenone (group D), 10 mg/kg finerenone (group E), 3 mg/kg empagliflozin+3 mg/kg finerenone combination (group F).

FIG. 2c: Influence of two dosages of the SGLT2 inhibitor empagliflozin (groups B and C), two dosages of the MR antagonist finerenone (groups D and E), and the combination of the low dosages of empagliflozin (SGLT2 inhibitor) and finerenone (group F), respectively, on the urinary potassium concentration of awake ZDF rats chronically treated with groups A to F, which were examined according to section B below, ‘assessment of the physiological effectiveness’ for 24 hours in metabolic cages. n=14-16 animals/per group. +: p<0.05 vs. combination group, ++: p<0.01 vs. combination group, +++: p<0.005 vs. combination group, ‘ns’ means not significant vs. combination group. S means solvent (group A). 3 mg/kg empagliflozin (group B), 10 mg/kg empagliflozin (group C), 3 mg/kg finerenone (group D), 10 mg/kg finerenone (group E), 3 mg/kg empagliflozin+3 mg/kg finerenone combination (group F).

FIG. 2d: Influence of two dosages of the SGLT2 inhibitor empagliflozin (groups B and C), two dosages of the MR antagonist finerenone (groups D and E), and the combination of the low dosages of empagliflozin (SGLT2 inhibitor) and finerenone (group F), respectively, on the urinary sodium concentration (FIG. 2d) of awake ZDF rats chronically treated with groups A to F, which were examined according to section B below, ‘assessment of the physiological effectiveness’ for 24 hours in metabolic cages. n=14-16 animals/per group. +: p<0.05 vs. combination group, ++: p<0.01 vs. combination group, +++: p<0.005 vs. combination group, ‘ns’ means not significant vs. combination group. S means solvent (group A). 3 mg/kg empagliflozin (group B), 10 mg/kg empagliflozin (group C), 3 mg/kg finerenone (group D), 10 mg/kg finerenone (group E), 3 mg/kg empagliflozin+3 mg/kg finerenone combination (group F).

FIG. 3a: Mortality was studied in hypertensive and proteinuric L-NAME (20 mg/L) treated renin-transgenic (mRen2)27 rats.

FIG. 3b: Proteinuria was studied in hypertensive and proteinuric L-NAME (20 mg/L) treated renin-transgenic (mRen2)27 rats.

FIG. 4: Influence of the SGLT2 inhibitor canagliflozin (groups B-D), of the MR antagonist finerenone (E), and their combination (groups F-H) on (a) urinary volume (FIG. 4a), (b) the urinary glucose concentration (FIG. 4b), (c) the urinary potassium concentration (FIGS. 4c), and (d) the urinary sodium concentration (FIG. 4d), of awake Wistar rats with activated renin-angiotensin-aldosterone system, which were examined according to section B below, ‘assessment of the physiological effectiveness’ for 24 hours after oral administration of the substances in metabolic cages. n=9-10 animals/per group. +: p<0.05 vs. combination group, ++: p<0.01 vs. combination group, +++: p<0.005 vs. combination group, ‘ns’ means not significant vs. combination group. S means solvent (group A). 1 mg/kg canagliflozin (group B), 3 mg/kg canagliflozin (group C), 10 mg/kg canagliflozin (group D), 1 mg/kg finerenone (group E), 1 mg/kg canagliflozin+1 mg/kg finerenone combination (group F), 3 mg/kg canagliflozin+1 mg/kg finerenone combination (group G), 10 mg/kg canagliflozin+1 mg/kg finerenone combination (group H).

FIG. 5: Influence of the SGLT2 inhibitor dapagliflozin (groups B-D), of the MR antagonist finerenone (E), and their combination (groups F-H) on (a) urinary volume (FIG. 5a), (b) the urinary glucose concentration (FIG. 5b), (c) the urinary potassium concentration (FIGS. 5c), and (d) the urinary sodium concentration (FIG. 5d), of awake Wistar rats with activated renin-angiotensin-aldosterone system, which were examined according to section B below, ‘assessment of the physiological effectiveness’ for 24 hours after oral administration of the substances in metabolic cages. n=10 animals/per group. +: p<0.05 vs. combination group, ++: p<0.01 vs. combination group, +++: p<0.005 vs. combination group, ‘ns’ means not significant vs. combination group. S means solvent (group A). 0.03 mg/kg dapagliflozin (group B), 0.3 mg/kg dapagliflozin (group C), 3 mg/kg dapagliflozin (group D), 1 mg/kg finerenone (group E), 0.03 mg/kg dapagliflozin+1 mg/kg finerenone combination (group F), 0.3 mg/kg dapagliflozin+1 mg/kg finerenone combination (group G), 3 mg/kg dapagliflozin+1 mg/kg finerenone combination (group H).

### EXAMPLES

### A—Pharmaceutical Formulation/Dosage Form

### A-1-1: Tablet Comprising Finerenone (4S)-4-(4-cyano-2-methoxyphenyl)-5-ethoxy-2,8-dimethyl-1,4-dihydro-1,6-naphthyridine-3-carboxamide of the Formula (I)

A granulate solution of the compound of formula (I) in crystalline form in micronized form, hypromellose 5 cP, sodium lauryl sulfate in purified water was prepared. Microcrystalline cellulose, lactose monohydrate and croscarmellose sodium were mixed in a container or a fluidized bed granulator (premix). The premix and the granulate solution were granulated in the fluid-bed granulator. The lubricant magnesium stearate was added after the granules were dried and sieved. A ready-to-press mixture was thus produced. The ready-to-press mixture was pressed into tablets using a rotary tablet press.

A homogeneous coating suspension was made from hypromellose, talc, titanium dioxide, yellow iron oxide, red iron oxide and purified water. The coating suspension was sprayed onto the tablets in a suitable coating device.

TABLE 1-1
Tablets (number 1 to 7) obtained by the process described above

| | | Tablets | | | | | | |
|---|---|---|---|---|---|---|---|---|
| | | 1 | 2 | 3 | 4 | 5 | 6 | 7 |
| Composition | | [mg] | [mg] | [mg] | [mg] | [mg] | [mg] | [mg] |
| | Finerenone, | 1.25 | 2.50 | 5.00 | 7.50 | 10.00 | 15.00 | 20.00 |
| | micronized | | | | | | | |
| Excipients | Cellulose | 73.80 | 72.50 | 69.90 | 67.30 | 64.70 | 62.00 | 59.30 |
| | microcrystalline | | | | | | | |
| | Crosscarmellose | 4.50 | 4.50 | 4.50 | 4.50 | 4.50 | 4.50 | 4.50 |
| | sodium | | | | | | | |
| | Hypromellose 5 | 4.50 | 4.50 | 4.50 | 4.50 | 4.50 | 4.50 | 4.50 |
| | cP | | | | | | | |
| | Lactose | 45.00 | 45.00 | 45.00 | 45.00 | 45.00 | 42.50 | 40.00 |
| | monohydrate | | | | | | | |
| | Magnesium | 0.90 | 0.90 | 0.90 | 0.90 | 0.90 | 0.90 | 0.90 |
| | stearate | | | | | | | |
| | Sodium | 0.05 | 0.10 | 0.20 | 0.30 | 0.40 | 0.60 | 0.80 |
| | laurilsulfate | | | | | | | |
| | Weight | 130.00 | 130.00 | 130.00 | 130.00 | 130.00 | 130.00 | 130.00 |
| | (uncoated | | | | | | | |
| | tablet) | | | | | | | |
| Film- | Hypromellose 5 | 3.0336 | 3.0336 | 3.0336 | 3.0336 | 3.0336 | 3.0336 | 3.0336 |
| coating | cP | | | | | | | |
| | Titanium | 2.3196 | 2.3196 | 2.3196 | 2.3196 | 2.3196 | 2.3196 | 2.3196 |
| | dioxide | | | | | | | |
| | Talcum | 0.6072 | 0.6072 | 0.6072 | 0.6072 | 0.6072 | 0.6072 | 0.6072 |
| | Iron oxide | 0.0324 | 0.0324 | 0.0324 | 0.0324 | 0.0324 | 0.0324 | 0.0324 |
| | yellow | | | | | | | |
| | Iron oxide | 0.0072 | 0.0072 | 0.0072 | 0.0072 | 0.0072 | 0.0072 | 0.0072 |
| | red | | | | | | | |
| | Weight | 6.0000 | 6.0000 | 6.0000 | 6.0000 | 6.0000 | 6.0000 | 6.0000 |
| | (film-coating) | | | | | | | |
| | Weight | 136.00 | 136.00 | 136.00 | 136.00 | 136.00 | 136.00 | 136.00 |
| | (coated tablet) | | | | | | | |

### A-1-2: Tablets Comprising Finerenone (4S)-4-(4-cyano-2-methoxyphenyl)-5-ethoxy-2,8-dimethyl-1,4-dihydro-1,6-naphthyridine-3-carboxamide of the Formula (I)

A granulate suspension of the compound of formula (I) in crystalline form in micronized form, hypromellose, sodium lauryl sulfate in purified water was prepared. Microcrystalline cellulose, lactose monohydrate and croscarmellose sodium were mixed in a container or a fluidized bed granulator (premix). The premix and the granulate solution were granulated in the fluid-bed granulator. The lubricant magnesium stearate was added after the granules were dried and sieved. A ready-to-press mixture was thus produced. The ready-to-press mixture was compressed into tablets using a rotary tablet press. A homogeneous coating suspension was made from hypromellose, talc, titanium dioxide, yellow iron oxide, red iron oxide and/or black iron oxide and purified water. The coating suspension was sprayed onto the tablets in a suitable coating device. The composition of the tablets obtained by the process described are listed in table 1-2.

TABLE 1-2
Tablets (8 to 12 obtained) obtained by the process described above

| | | Tablets | | | | |
|---|---|---|---|---|---|---|
| | | 8 | 9 | 10 | 11 | 12 |
| Composition | | [mg] | [mg] | [mg] | [mg] | [mg] |
| | Finerenone | 40 | 5 | 10 | 20 | 40 |
| | micronized | | | | | |
| Excipients | Cellulose | 110 | 69.9 | 64.7 | 59.3 | 110 |
| | microcrystalline | | | | | |
| | Croscarmellose | 15 | 4.5 | 4.5 | 4.5 | 15 |
| | sodium | | | | | |
| | Hypromellose 5 | 7 | 4.5 | 4.5 | 4.5 | 7 |
| | cP | | | | | |
| | Lactose | 25 | 45 | 45 | 40 | 25 |
| | monohydrate | | | | | |
| | Magnesium | 1.4 | 0.9 | 0.9 | 0.9 | 1.4 |
| | stearate | | | | | |
| | Sodium | 1.6 | 0.2 | 0.4 | 0.8 | 1.6 |
| | laurilsulfate | | | | | |
| | Purified water in | q.s. | q.s. | q.s. | q.s. | q.s. |
| | bulk | | | | | |
| | Weight | 200 | 130 | 130 | 130 | 200 |
| | (uncoated tablet) | | | | | |
| Film | Hypromellose 5 | 3.5392 | 3 | 3 | 3 | 3.5 |
| coating | cP | | | | | |
| | Talc | 0.7084 | 0.6 | 0.6 | 0.6 | 0.7 |
| | Titanium dioxide | 2.7062 | 2.36 | 2.28 | 1.92 | 2.222 |
| | Ferric oxide | 0.0378 | — | — | 0.48 | 0.473 |
| | yellow | | | | | |
| | Ferric oxide red | 0.0084 | — | 0.12 | — | 0.105 |
| | Ferric oxide black | — | 0.04 | — | — | — |
| | Purified water in | q.s. | q.s. | q.s. | q.s. | q.s. |
| | bulk | | | | | |
| | Weight (film | 7 | 6 | 6 | 6 | 7 |
| | coating) | | | | | |

| | | | | | |
|---|---|---|---|---|---|
| Weight | 207 | 136 | 136 | 136 | 207 |
| (coated tablet) | | | | | |

As film coatings, commercially available film coatings can be used for the tablets disclosed in tables 1-1 and 1-2 above. Examples are Opadry® film coatings such as Opadry® 02A275000 light gray, Opadry® 02A240005 light pink or Opadry® 02A220009 light yellow.

### B—Assessment of Physiological Effectiveness

### B-1 In Vivo Assay for Detecting Natriuretic Activity in Conscious Rats with Activated RAAS

Wistar rats (ca. 250-500 g body weight) were kept with free access to feed (Altromin) and drinking water. From approx. 72 hours before the start of the test, the animals received, instead of the normal feed, exclusively reduced-salt feed with a sodium chloride content of 0.02% Sodium Chloride (ssniff R/M-H, 10 mm with 0.02% Na, S0602-E081, ssniff Spezialdiaten GmbH, D-59494 Soest). This sodium-reduced feed causes an activation of the RAAS in the animals during the 3 days run-in phase and therefore mimics a neuroendocrine activation which is characteristic for cardiorenal diseases. During the test, the animals were housed singly in metabolic cages suitable for rats of this weight class (Tecniplast Deutschland GmbH, D-82383 Hohenpeissenberg) with free access to reduced-salt feed and drinking water for up to 24 hours. At the start of the test, the substance to be tested was administered by means of gavage in a volume of 2-3 ml/kg of body weight of a suitable solvent PEG400). Control animals received only solvent (‘S’ or solvent in table 2 below). Controls and substance tests were carried out in parallel on the same day. Control groups and substance-dose groups each consisted of 6 to 10 animals. During the test, the urine excreted by the animals was continuously collected in a receiver on the base of the cage. The urine volume per collection time was determined separately for each animal, and the concentration of glucose, sodium and potassium excreted in the urine was measured by standard methods using a clinical-chemical analyzer system (ADVIA 2400, Siemens). The urine was typically collected for 24 hours in the metabolic cages.

### B-2 In Vivo Assay for Detecting Natriuretic Activity in Chronically Treated Zucker Diabetic Fatty Rats

The Zucker diabetic fatty (ZDF) rat has a missense mutation in the gene coding the leptin receptor (fa/fa) and spontaneously develops insulin resistance, type 2 diabetes mellitus (T2DM), hyperlipidemia, moderate hypertension, and obesity, as well as progressive renal injury. Male homozygous animals develop diabetes mellitus from week 7 to 19 which is reflected in marked hyperglycemia. In addition to diabetic cardiac lesions (e.g. hypertrophy), renal pathology develops with proteinuria, mesangial expansion, macrophage infiltration, and interstitial fibrosis. Heterozygous animals are useful non-diabetic control animals since they develop neither obesity nor insulin resistance.

Male 6-7 weeks old obese (fa/fa) Zucker rats (Charles River) were placed on a high energy diet (Purina Rodent LabDiet 5008, PMI Nutrition. Richmond. Ind.) and were randomized to treatment groups or solvent group when the animals were hyperglycemic. Rats (n=14-16/group) received either solvent [ethanol/Solutol/H₂O (10/40/50)] or test compounds in solvent once daily applied to (fa/fa) animals for 4 to 12 weeks.

During the experiment, the systolic blood pressure (determined by tail cuff), urine parameters (e.g. protein, glucose, electrolytes, creatinine, urea and uric acid) and blood plasma parameters (e.g. electrolytes, glucose, creatinine, urea and uric acid) were determined at regular intervals.

For the determination of urinary parameter the animals were housed singly in metabolic cages suitable for rats of this weight class (Tecniplast Deutschland GmbH, D-82383 Hohenpeissenberg) with free access to drinking water for up to 24 hours. The urine volume per collection time was determined separately for each animal, and the concentration of urinary glucose and electrolyte ions excreted in the urine was measured by standard methods by a clinical-chemical analyzer system (ADVIA 2400, Siemens). The urine was typically collected for 24 hours in metabolic cages. At the end of the chronic experiment, hemodynamic parameter (e.g. blood pressure, heart rate, maximum and minimum inotropy [dp/dt], relaxation time [tau], left ventricular pressure, left ventricular end diastolic pressure [LVEDP]) are measured, and the weights of heart, kidney and lung are determined, plasma and urine biomarkers (e.g. NT-proBNP) and gene expression of biomarkers by RT/TaqMan PCR following RNA isolation from cardiac and renal tissue are determined. Histopathology is performed with cardiac and renal tissue from animals of treatment groups and placebo groups.

### B-3 Comparison of Monotherapy (Finerenone or Empagliflozin (SGLT2 Inhibitor)) with Combination Therapy (Combined Use of Finerenone and Empagliflozin (SGLT2 Inhibitor)) in Conscious Rats with Activated RAAS

In this comparison, eight different administrations were performed (cf. groups A to H):

Group A received the solvent (PEG400) only. This group serves as control group.

Groups B, C and D received three increasing dosages of empagliflozin (SGLT2 inhibitor) only. It serves to detect the effects of the monotherapy with SGLT2 inhibitor.

Group E received a finerenone only. It serves to detect the effect of the monotherapy with finerenone.

Groups F, G and H received finerenone and the three doses of SGLT2 inhibitor in combination, respectively. It serves to detect the effect of the combined therapy with finerenone and empagliflozin (SGLT2 inhibitor) (combination according to the invention).

After the treatment, the urinary volume, urinary glucose, urinary sodium and potassium concentrations were measured. Table 2 summarizes the results.

TABLE 2
Measured data (mean values ± standard error [SE]) of urinary volume, urinary
glucose, urinary potassium concentration (K+), and urinary sodium concentration (Na+),
from awake Wistar rats with activated renin-angiotensin-aldosterone system (n = 6-8
animals per group), which were examined according to the ‘assessment of the physiological
efficacy’ for 24 hours after oral administration of the substances in metabolic cages.
‘b.d.l.’ means below detection limit of glucose in urine.

| | Urinary Volume/ | Urinary Glucose | Urinary K+ | Urinary Na+ |
|---|---|---|---|---|
| | body weight | concentration | concentration | concentration |
| Group | [ml/kg] | [mmol] | [mmol] | [mmol] |
| A: Solvent | 32.25 ± 7.44 | b.d.l. | 2.21 ± 0.32 | 0.19 ± 0.03 |
| Mean value ± SEM | | | | |
| B: 1 mg/kg empagliflozin | 27.80 ± 3.58 | 0.34 ± 0.10 | 1.66 ± 0.11 | 0.15 ± 0.05 |
| (SGLT2 inhibitor) | | | | |
| (Monotherapy) | | | | |
| Mean value ± SEM | | | | |
| C: 3 mg/kg empagliflozin | 22.96 ± 2.75 | 1.21 ± 0.21 | 1.74 ± 0.15 | 0.13 ± 0.02 |
| (SGLT2 inhibitor) | | | | |
| (Monotherapy) | | | | |
| Mean value ± SEM | | | | |
| D: 10 mg/kg empagliflozin | 28.45 ± 1.91 | 4.26 ± 0.59 | 1.85 ± 0.11 | 0.17 ± 0.03 |
| (SGLT2 inhibitor) | | | | |
| (Monotherapy) | | | | |
| Mean value ± SEM | | | | |
| E: 1 mg/kg Finerenone | 21.90 ± 1.86 | b.d.l. | 1.46 ± 0.12 | 0.43 ± 0.03 |
| (Monotherapy) | | | | |
| Mean value ± SEM | | | | |
| F: Combination group | 23.47 ± 3.47 | 1.11 ± 0.46 | 1.62 ± 0.27 | 0.71 ± 0.07 |
| 1 mg/kg empagliflozin | | p < 0.05 vs. E | | p < 0.005 vs. B |
| (SGLT2 inhibitor) + | | | | p < 0.01 vs. E |
| 1 mg/kg Finerenone | | | | |
| Mean value ± SEM | | | | |
| G: Combination group | 33.96 ± 2.79 | 1.54 ± 0.30 | 1.58 ± 0.14 | 0.96 ± 0.09 |
| 3 mg/kg empagliflozin | p < 0.05 vs. C | p < 0.005 vs. E | | p < 0.005 vs. C |
| (SGLT2 inhibitor) + | p < 0.01 vs. E | | | p < 0.005 vs. E |
| 1 mg/kg Finerenone | | | | |
| Mean value ± SEM | | | | |
| H: Combination group | 36.76 ± 2.19 | 4.79 ± 0.50 | 1.75 ± 0.16 | 0.89 ± 0.09 |
| 10 mg/kg empagliflozin | p < 0.05 vs. D | p < 0.005 vs. E | | p < 0.005 vs. D |
| (SGLT2 inhibitor) + 1 mg/kg | p < 0.005 vs. E | | | p < 0.005 vs. E |
| Finerenone | | | | |
| Mean value ± SEM | | | | |

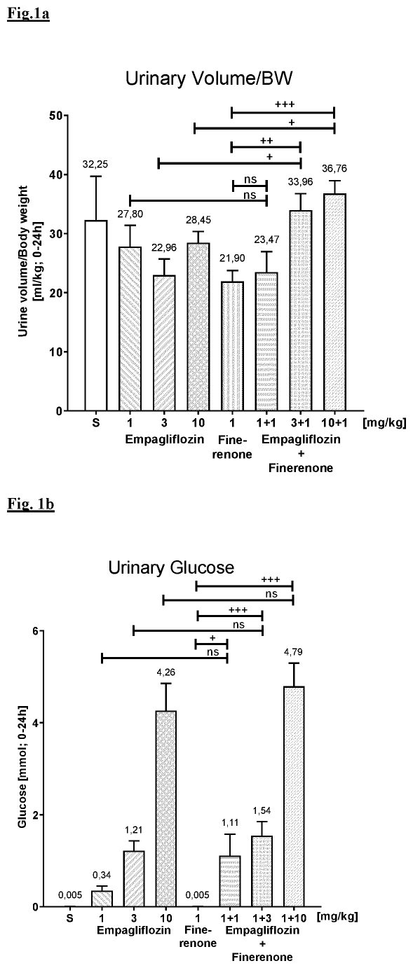

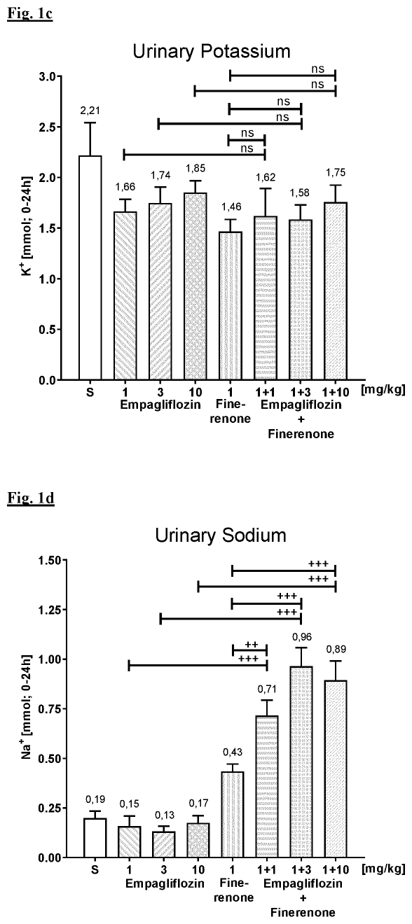

The results are also depicted in FIG. 1.

FIG. 1: Influence of the SGLT2 inhibitor empagliflozin (groups B-D), of the MR antagonist finerenone (E), and their combination (groups F-H) on
(a) urinary volume (FIG. 1a),
(b) the urinary glucose concentration (FIG. 1b),
(c) the urinary potassium concentration (FIG. 1c),
(d) the urinary sodium concentration (FIG. 1d), and
   of awake Wistar rats with activated renin-angiotensin-aldosterone system, which were examined according to section B above, ‘assessment of the physiological effectiveness’ for 24 hours after oral administration of the substances in metabolic cages. n=6-8 animals/per group. +: p<0.05 vs. combination group, ++: p<0.01 vs. combination group, +++: p<0.005 vs. combination group, ‘ns’ means not significant vs. combination group. S means solvent (group A). 1 mg/kg empagliflozin (group B), 3 mg/kg empagliflozin (group C), 10 mg/kg empagliflozin (group D), 1 mg/kg finerenone (group E), 1 mg/kg empagliflozin+1 mg/kg finerenone combination (group F), 3 mg/kg empagliflozin+1 mg/kg finerenone combination (group G), 10 mg/kg empagliflozin+1 mg/kg finerenone combination (group H).

From table 2 and FIG. 1 a to d the following can be concluded:

Group A (solvent only), in which the solvent (PEG400) was administered, shows the physiological excretion of volume, potassium and sodium of conscious rats over 24 hours under RAAS activation.

Groups B, C and D (monotherapy empagliflozin (SGLT2 inhibitor)) show that administration of empagliflozin has no influence on urinary volume, urinary potassium and urinary sodium at the indicated doses but leads to a dose-dependent and strong increase in urinary glucose.

Group E (monotherapy finerenone) shows that administration of 1 mg/kg of finerenone has no influence on urinary volume, urinary glucose and urinary potassium, but causes an increase in urinary sodium.

Groups F, G and H (combination of finerenone and empagliflozin (SGLT2 inhibitor)) show that administration of a combination of 1 mg/kg finerenone and 1 to 10 mg/kg empagliflozin (SGLT2 inhibitor) does not increase urinary glucose and urinary potassium in comparison to the respective individual finerenone and empagliflozin (SGLT2 inhibitor) dosage, but increases urinary volume at the two higher combination dosages and dose-dependently and strongly induces sodium excretion in all combination groups in comparison to the respective individual finerenone and empagliflozin (SGLT2 inhibitor).

A comparison of group A (solvent) with groups F, G and H (combination finerenone and empagliflozin (SGLT2 inhibitor)) shows that administration of a combination of finerenone and empagliflozin (SGLT2 inhibitor) that chronic administration of a combination of finerenone and the SGLT2 inhibitor increases the urinary sodium excretion from 1.16±0.06 mmol (group A) to 0.71±0.07 mmol (group F, combination), 0.96±0.09 mmol (group G, combination), and 0.89±0.09 mmol (group H, combination),

A comparison of groups B, C, D and E (monotherapy with empagliflozin or finerenone) with groups F, G and H (combination finerenone and empagliflozin (SGLT2 inhibitor)) shows that administration of a combination of finerenone and empagliflozin (SGLT2 inhibitor) that chronic administration of a combination of finerenone and the SGLT2 inhibitor increases the urinary sodium excretion from 0.15±0.05 mmol (group B, monotherapy empagliflozin), 0.13±0.02 mmol (group C, monotherapy empagliflozin), 0.17±0.03 mmol (group D, monotherapy empagliflozin), 0.43±0.03 mmol (group E, monotherapy empagliflozin), to 0.71±0.07 mmol (group F, combination), 0.96±0.09 mmol (group G, combination), and 0.89±0.09 mmol (group H, combination). This is surprising as the combination of finerenone and empagliflozin (SGLT2 inhibitor) shows an over-additive effect.

Note that the urinary sodium concentration is statistically significant higher. The urinary sodium excretion of the combination therapy is more than the pure sum of the respective monotherapies. Thereby showing an over-additive effect in comparison to the respective monotherapy. Therefore, the combination of finerenone and empagliflozin (SGLT2 inhibitor) under typical conditions of activated RAAS leads to a significant natriuretic efficacy improvement over the sum of the monotherapies. This natriuretic efficacy improvement is a major clinical goal in the treatment of cardiovascular and/or cardiorenal diseases such as heart failure and CKD.

### B-4 Comparison of Monotherapy (Finerenone or a SGLT2 Inhibitor) with Combination Therapy (Combined Use of Finerenone and a SGLT2 Inhibitor) in Chronically Treated Zucker Diabetic Fatty Rats

In this comparison, six different administrations were performed:

Group A received the solvent [ethanol/Solutol/H₂O (10/40/50)] only. This group serves as control group.

Groups B and C received two increasing dosages of empagliflozin (SGLT2 inhibitor) only. It serves to detect the effects of the monotherapy with empagliflozin (SGLT2 inhibitor).

Groups D and E received increasing dosages of finerenone only. It serves to detect the effect of the monotherapy with finerenone.

Group F received a combination of the low dosages of empagliflozin (SGLT2 inhibitor) and finerenone, respectively. It serves to detect the effect of the combined therapy with finerenone and empagliflozin (SGLT2 inhibitor) (combination according to the invention).

After the treatment, the urinary volume, urinary glucose, urinary sodium and potassium concentrations were measured. Table 3 summarizes the results.

TABLE 3
Measured data (mean values ± standard error [SE]) of urinary volume, urinary
glucose, urinary potassium concentration (K+), and urinary sodium concentration (Na+),
from awake diabetic ZDF rats chronically treated for 9 weeks with either solvent, two
dosages of SGLT2 inhibitor, two dosages of finerenone, or a combination of the low dosages
of SGLT2 inhibitor and finerenone (n = 14-16 animals per group), which were examined
according to the ‘assessment of the physiological efficacy’ for 24 hours in metabolic cages.

| | Urinary Volume/ | Urinary Glucose | Urinary K+ | Urinary Na+ |
|---|---|---|---|---|
| | body weight | concentration | concentration | concentration |
| Group | [ml/kg] | [mmol] | [mmol] | [mmol] |
| A: Solvent | 63.77 ± 2.82 | 249.65 ± 20.96 | 1.95 ± 0.08 | 1.16 ± 0.06 |
| Mean value ± SEM | | | | |
| B: 3 mg/kg | 99.98 ± 7.12 | 321.55 ± 22.78 | 2.22 ± 0.13 | 1.47 ± 0.10 |
| empagliflozin (SGLT2 | | | | |
| inhibitor) | | | | |
| (Monotherapy) | | | | |
| Mean value ± SEM | | | | |
| C: 10 mg/kg | 110.38 ± 4.55 | 317.16 ± 25.10 | 2.64 ± 0.20 | 2.05 ± 0.27 |
| empagliflozin (SGLT2 | | | | |
| inhibitor) | | | | |
| (Monotherapy) | | | | |
| Mean value ± SEM | | | | |
| D: 3 mg/kg Finerenone | 81.84 ± 11.99 | 291.98 ± 19.11 | 1.94 ± 0.14 | 1.39 ± 0.12 |
| (Monotherapy) | | | | |
| Mean value ± SEM | | | | |
| E: 10 mg/kg | 56.82 ± 2.59 | 272.62 ± 22.51 | 1.75 ± 0.06 | 1.30 ± 0.08 |
| Finerenone | | | | |
| (Monotherapy) | | | | |
| Mean value ± SEM | | | | |
| F: Combination group | 109.12 ± 3.30 | 338.87 ± 10.86 | 2.13 ± 0.10 | 2.07 ± 0.15 |
| 3 mg/kg empagliflozin | p < 0.05 vs. D | p < 0.05 vs. D | | p < 0.01 vs. B |
| (SGLT2 inhibitor) + 3 | | | | p < 0.01 vs. D |
| mg/kg Finerenone | | | | |
| Mean value ± SEM | | | | |

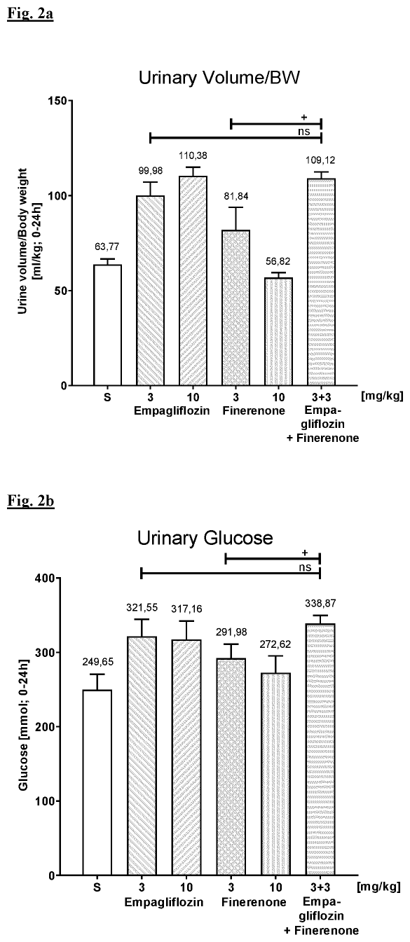

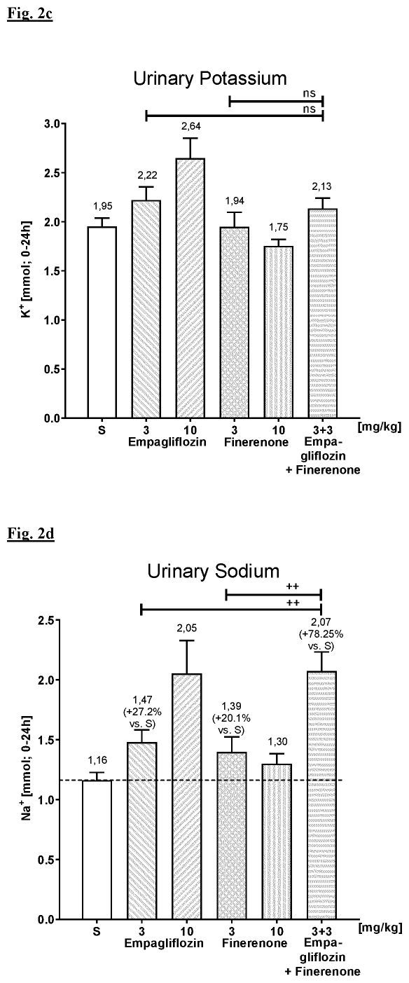

The results are also depicted in FIG. 2.

FIG. 2: Influence of two dosages of the SGLT2 inhibitor empagliflozin (groups B and C), two dosages of the MR antagonist finerenone (groups D and E), and the combination of the low dosages of empagliflozin (SGLT2 inhibitor) and finerenone (group F), respectively, on

(a) urinary volume (FIG. 2a),

(b) the urinary glucose concentration (FIG. 2b),

(c) the urinary potassium concentration (FIG. 2c),

(d) the urinary sodium concentration (FIG. 2d), and

of awake ZDF rats chronically treated with groups A to F, which were examined according to section B above, ‘assessment of the physiological effectiveness’ for 24 hours in metabolic cages. n=14-16 animals/per group. +: p<0.05 vs. combination group, ++: p<0.01 vs. combination group, +++: p<0.005 vs. combination group, ‘ns’ means not significant vs. combination group. S means solvent (group A). 3 mg/kg empagliflozin (group B), 10 mg/kg empagliflozin (group C), 3 mg/kg finerenone (group D), 10 mg/kg finerenone (group E), 3 mg/kg empagliflozin+3 mg/kg finerenone combination (group F).

From table 3 and FIG. 2 (a) to (d) the following can be concluded:

Group A (solvent), in which the solvent ([ethanol/Solutol/H₂O (10/40/50)]) was administered, shows the excretion of volume, glucose, potassium and sodium of conscious Zucker diabetic fatty rats over 24 hours after a treatment period of 9 weeks.

A comparison of group A (solvent) with groups B and C (monotherapy empagliflozin (SGLT2 inhibitor)) show that chronic administration of a SGLT2 inhibitor increases urinary volume, urinary glucose, urinary potassium and urinary sodium. When comparing the solvent group A with the empagliflozin (SGLT2 inhibitor) monotherapy it can be seen that the urinary sodium excretion is 27.2% higher in comparison to solvent control group A.

A comparison of group A (solvent) with groups D and E (monotherapy finerenone) shows that chronic administration of finerenone has no influence on urinary volume, urinary glucose, urinary potassium and urinary sodium at the indicated dosages of 3 and 10 mg/kg/day. When comparing the solvent group A with the finerenone monotherapy it can be seen that the urinary sodium excretion is 20.1% higher in comparison to solvent control group A.

A comparison of the low dosage groups B (3 mg/kg empagliflozin (SGLT2 inhibitor), monotherapy) and D (3 mg/kg finerenone, monotherapy) with group F (combination finerenone and empagliflozin (SGLT2 inhibitor)) shows that administration of a combination of finerenone and empagliflozin (SGLT2 inhibitor) does not increase urinary volume, glucose and urinary potassium in comparison to the respective individual finerenone and SGLT2 inhibitor dosages, but leads to a statistically significant increase in urinary sodium excretion that is approx. 41 to approx. 58% higher in comparison to the respective monotherapies. This is surprising as the combination of finerenone and empagliflozin (SGLT2 inhibitor) shows an over-additive effect.

In summary, the urinary sodium excretion is 27.2% and 20.1% higher for the 3 mg/kg empagliflozin (SGLT2 inhibitor) and 3 mg/kg finerenone dosages, respectively, in comparison to the solvent control (group A), while the combined urinary sodium excretion of the combination of finerenone and empagliflozin is 78.25% higher in comparison to solvent control group A and, thus, more than the sum of the respective monotherapies. Thus, in comparison to the respective monotherapies, the sodium excretion of the combination is approx. 41 to approx. 58% higher.

Therefore, the combination of finerenone and empagliflozin (SGLT2 inhibitor) under typical chronic conditions including insulin resistance, type 2 diabetes mellitus, hyperlipidemia, hypertension, and obesity, as well as progressive renal injury leads to a significant natriuretic efficacy improvement over the sum of the monotherapies. This natriuretic efficacy improvement is a major clinical goal in the treatment of cardiovascular and/or cardiorenal diseases such as heart failure and CKD.

### B-5 Combined Efficacy of Nonsteroidal MR Antagonist Finerenone and SGLT2 Inhibitor Empagliflozin in a Non-Diabetic Cardiorenal Rat Model

Method:

Cardiorenal morbidity and mortality was studied in hypertensive and proteinuric L-NAME (20 mg/L) treated renin-transgenic (mRen2)27 rats. Rats (10-11 weeks old female, n=13-17/group) were treated once daily orally for up to 7 weeks with placebo, finerenone (1 and 3 mg/kg), empagliflozin (3 and 10 mg/kg), or a combination of the respective low doses. Key outcome parameters included mortality, blood pressure, proteinuria, kidney histology and gene expression.

L-NAME-Treated Transgenic Renin Rat (TGR(mRen2)27):

The transgenic renin rat ‘TGR(mRen2)27’ is a hypertensive rat line developed by Mullins and Ganten which overexpresses the Ren-2 gene of the mouse. Additional administration of the nitrogen monoxide synthase inhibitor L-NAME induces endothelial dysfunction which increases morbidity and mortality in this model. Unless subjected to life-long antihypertensive therapy, homozygous animals die of secondary complications such as heart and kidney failure or stroke.

Female TGR(mRen2)27 renin rats aged 10 to 20 weeks are randomized to different pharmacological treatment groups and a placebo group. In addition, the nitrogen monoxide synthase inhibitor L-NAME is administered via the drinking water in a concentration of 20 to 100 mg/l. During the entire experiment, drinking water and feed are available ad libitum to the animals. The substances are administered via the feed or daily by gavage for 4-10 weeks. Animals treated in the same way but receiving either only the solvent or the feed without test substance serve as placebo group. During the experiment, the systolic blood pressure is determined at regular intervals using a tail cuff, and proteinuria (expressed as ratio of urinary protein concentration per urinary creatinine concentration) and urine electrolyte composition are determined by collecting the urine in metabolic cages, and mortality is registered on a daily base. At the end of the experiment, haemodynamic parameters (blood pressure, heart rate, inotropism [dp/dt], relaxation time [tau], maximum left ventricular pressure, left ventricular end-diastolic pressure [LVEDP]) are measured, and the weights of heart, kidney and lung are determined, protein elimination and biomarkers (e.g. ANP, RIA Kit RK 005-24, Phoenix Pharmaceuticals, Inc., USA, cGMP, RIA Kit RE29075, IBL International GmbH, Hamburg, Germany, renin, angiotensin I, RIA Kit CA-1533, DiaSorin S.p.A., Italy, and aldosterone, P2714, DiaSorin S.p.A., Italy), renal and cardiac histopathology and gene expression of biomarkers by RT/TaqMan PCR following RNA isolation from cardiac and renal tissue are determined.

For the determination of urinary parameter the animals were housed singly in metabolic cages suitable for rats of this weight class (Tecniplast Deutschland GmbH, D-82383 Hohenpeissenberg) with free access to drinking water for up to 24 hours. The urine volume per collection time was determined separately for each animal, and the concentration of urinary glucose and electrolyte ions excreted in the urine was measured by standard methods by a clinical-chemical analyzer system (ADVIA 2400, Siemens). The urine was typically collected for 24 hours in metabolic cages.

Results:

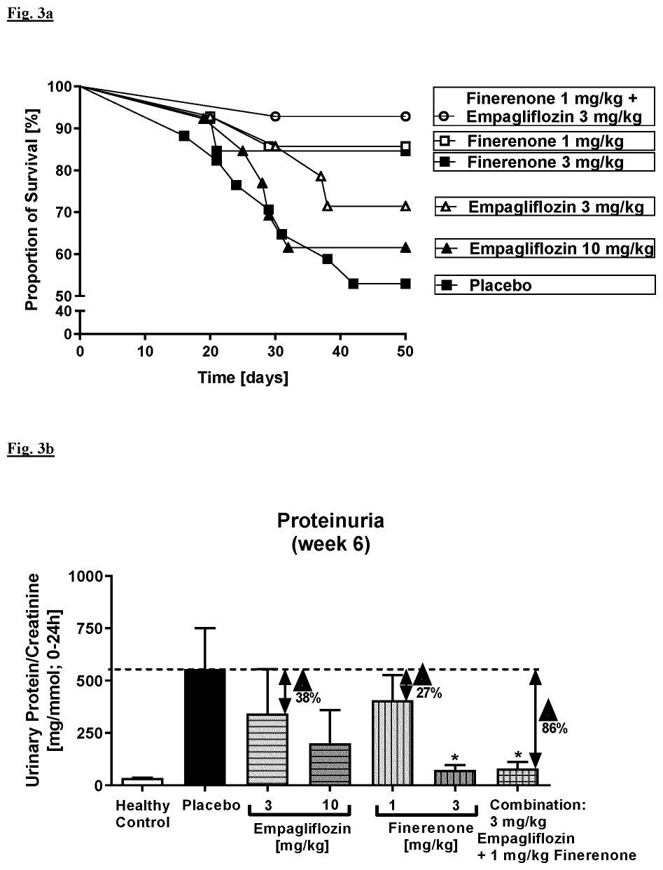

Placebo-treated rats demonstrated a 50% mortality rate over the course of 7 weeks (FIG. 3a). Drug treatment resulted in variable degrees of survival benefit, most prominent and statistically significant in the low dose combination group (FIG. 3a). Low dose combination revealed an early, sustained and efficacious proteinuria reduction (−86%, p<0.05; FIG. 3b) and was highly efficient on renal histology parameters. Monotherapies of finerenone (−27% at 1 mg/kg, p=n.s.; −87% at 3 mg/kg, p<0.05; FIG. 3b) and empagliflozin (−38% at 3 mg/kg, p=n.s.; −64% at 10 mg/kg, p=n.s.; FIG. 3b) dose-dependently reduced proteinuria with a comparable protection from renal lesions at higher dosages. Treatment with finerenone and the combination significantly decreased systolic blood pressure while empagliflozin alone and in combination acted strongly glucosoric.

Conclusion:

Both, MRA by finerenone and SGLT2 inhibitor by empagliflozin confer renal protection in preclinical non-diabetic, hypertensive kidney disease. Combination of these two modes of action at low dosages revealed efficacious reduction in proteinuria (see FIG. 3b) and mortality indicating a strong potential for combined clinical use in respective cardiorenal patient populations.

### B-6 Comparison of Monotherapy (Finerenone or Canagliflozin (SGLT2 Inhibitor)) with Combination Therapy (Combined Use of Finerenone and Canagliflozin (SGLT2 Inhibitor)) in Conscious Rats with Activated RAAS

In this comparison, eight different administrations were performed (cf. groups A to H):

Group A received the solvent (PEG400) only. This group serves as control group.

Groups B, C and D received three increasing dosages of canagliflozin (SGLT2 inhibitor) only. It serves to detect the effects of the monotherapy with SGLT2 inhibitor.

Group E received a finerenone only. It serves to detect the effect of the monotherapy with finerenone.

Groups F, G and H received finerenone and the three doses of SGLT2 inhibitor in combination, respectively. It serves to detect the effect of the combined therapy with finerenone and canagliflozin (SGLT2 inhibitor) (combination according to the invention).

The method as described in sections B1 was used.

TABLE 4

| | Urinary Volume/ | Urinary Glucose | Urinary K+ | Urinary Na+ |
|---|---|---|---|---|
| | body weight | concentration | concentration | concentration |
| Group | [ml/kg] | [mmol] | [mmol] | [mmol] |
| A: Solvent | 38.98 ± 4.23 | b.d.l. | 1.91 ± 0.13 | 0.10 ± 0.03 |
| Mean value ± SEM | | | | |
| B: 1 mg/kg canagliflozin | 31.08 ± 3.17 | 2.76 ± 0.36 | 1.86 ± 0.11 | 0.15 ± 0.03 |
| (SGLT2 inhibitor) | | | | |
| (Monotherapy) | | | | |
| Mean value ± SEM | | | | |
| C: 3 mg/kg canagliflozin | 40.49 ± 2.43 | 6.94 ± 0.44 | 2.40 ± 0.17 | 0.21 ± 0.05 |
| (SGLT2 inhibitor) | | | | |
| (Monotherapy) | | | | |
| Mean value ± SEM | | | | |
| D: 10 mg/kg canagliflozin | 55.83 ± 3.52 | 9.99 ± 0.46 | 2.36 ± 0.15 | 0.41 ± 0.08 |
| (SGLT2 inhibitor) | | | | |
| (Monotherapy) | | | | |
| Mean value ± SEM | | | | |
| E: 1 mg/kg finerenone | 29.32 ± 2.94 | b.d.l. | 1.76 ± 0.11 | 0.27 ± 0.06 |
| (Monotherapy) | | | | |
| Mean value ± SEM | | | | |
| F: Combination group | 34.17 ± 3.98 | 2.97 ± 0.28 | 1.71 ± 0.08 | 0.33 ± 0.04 |
| 1 mg/kg canagliflozin | | p < 0.005 vs. E | | p < 0.01 vs. B |
| (SGLT2 inhibitor) + | | | | |
| 1 mg/kg finerenone | | | | |
| Mean value ± SEM | | | | |
| G: Combination group | 48.40 ± 4.44 | 6.13 ± 0.61 | 1.83 ± 0.19 | 0.67 ± 0.06 |
| 3 mg/kg canagliflozin | p < 0.01 vs. E | p < 0.005 vs. E | p < 0.05 vs. C | p < 0.005 vs. C |
| (SGLT2 inhibitor) + | | | | p < 0.005 vs. E |
| 1 mg/kg finerenone | | | | |
| Mean value ± SEM | | | | |
| H: Combination group | 56.54 ± 4.09 | 9.30 ± 0.71 | 1.97 ± 0.14 | 0.96 ± 0.06 |
| 10 mg/kg canagliflozin | p < 0.005 vs. E | p < 0.005 vs. E | | p < 0.005 vs. D |
| (SGLT2 inhibitor) + | | | | p < 0.005 vs. E |
| 1 mg/kg finerenone | | | | |
| Mean value ± SEM | | | | |

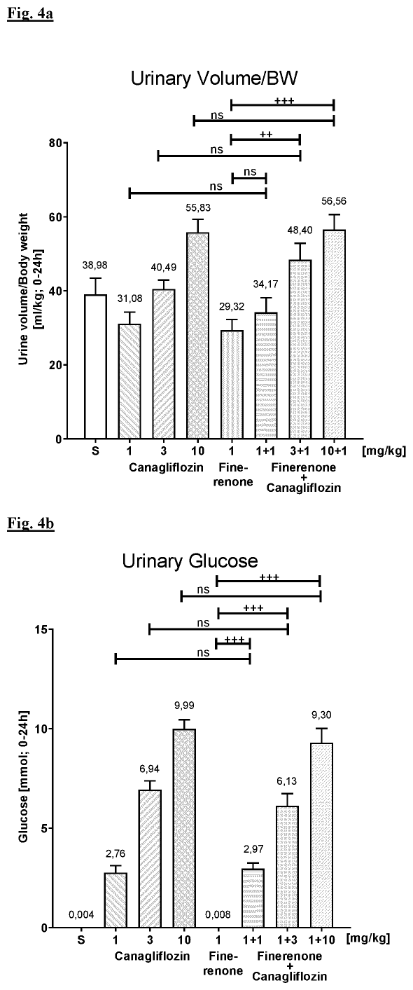

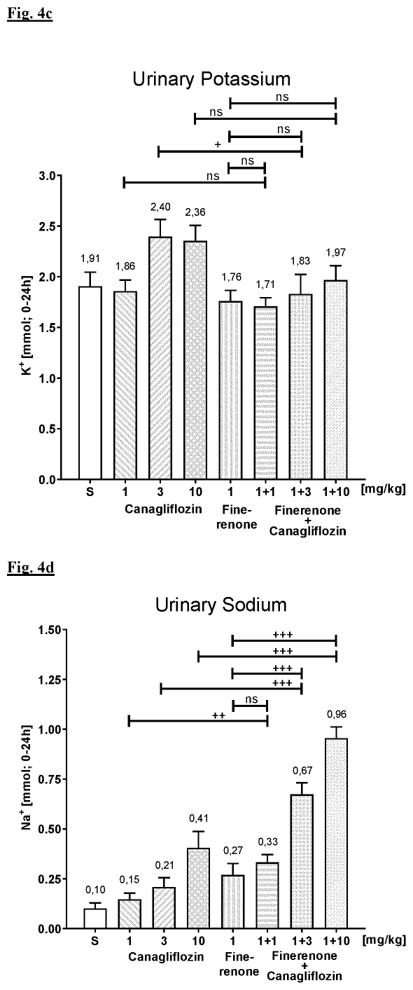

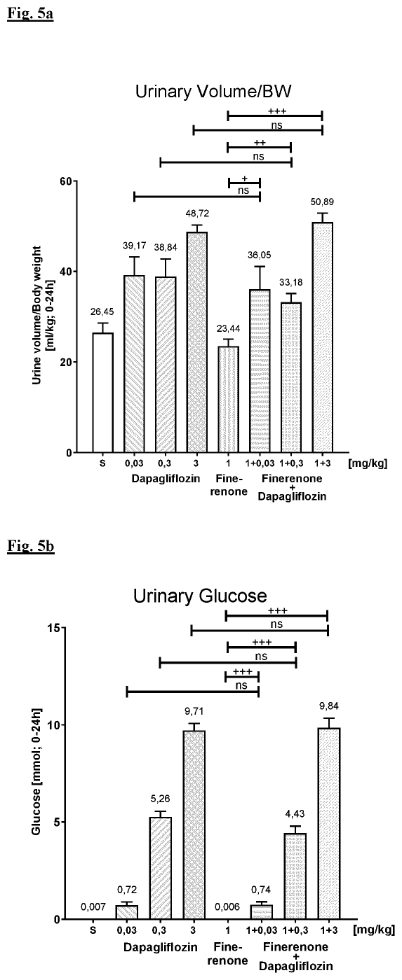

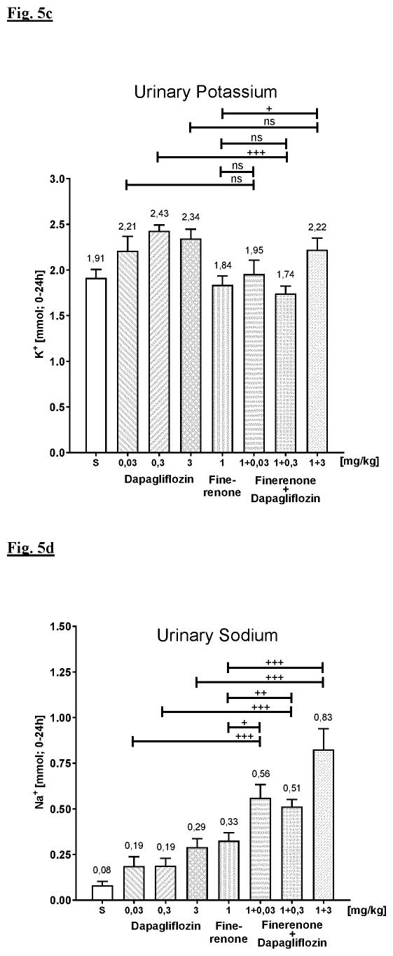

The results are also depicted in FIG. 4.

FIG. 4: Influence of the SGLT2 inhibitor canagliflozin (groups B-D), of the MR antagonist finerenone (E), and their combination (groups F-H) on

(a) urinary volume (FIG. 4a),

(b) the urinary glucose concentration (FIG. 4b),

(c) the urinary potassium concentration (FIG. 4c), and

(d) the urinary sodium concentration (FIG. 4d),

of awake Wistar rats with activated renin-angiotensin-aldosterone system, which were examined according to section B above, ‘assessment of the physiological effectiveness’ for 24 hours after oral administration of the substances in metabolic cages. n=9-10 animals/per group. +: p<0.05 vs. combination group, ++: p<0.01 vs. combination group, +++: p<0.005 vs. combination group, ‘ns’ means not significant vs. combination group. S means solvent (group A). 1 mg/kg canagliflozin (group B), 3 mg/kg canagliflozin (group C), 10 mg/kg canagliflozin (group D), 1 mg/kg finerenone (group E), 1 mg/kg canagliflozin+1 mg/kg finerenone combination (group F), 3 mg/kg canagliflozin+1 mg/kg finerenone combination (group G), 10 mg/kg canagliflozin+1 mg/kg finerenone combination (group H).

From table 4 and FIG. 4 a to d the following can be concluded:

Group A (solvent only), in which the solvent (PEG400) was administered, shows the physiological excretion of volume, potassium and sodium of conscious rats over 24 hours under RAAS activation.

Groups B, C and D (monotherapy canagliflozin (SGLT2 inhibitor)) show that administration of canagliflozin has no influence on urinary volume, urinary potassium and urinary sodium at the doses of 1 and 3 mg/kg while a dose of 10 mg/kg induces an increase in urinary volume and urinary sodium. All doses of canagliflozin induce a strong increase in urinary glucose.

Group E (monotherapy finerenone) shows that administration of 1 mg/kg of finerenone has no influence on urinary volume, urinary glucose and urinary potassium, but causes a weak increase in urinary sodium.

Groups F, G and H (combination of finerenone and canagliflozin (SGLT2 inhibitor)) show that administration of a combination of 1 mg/kg finerenone and 1 to 10 mg/kg canagliflozin does not increase urinary glucose in comparison to the respective individual canagliflozin dosages (finerenone alone has no effect on urinary glucose), and does not increase urinary volume in comparison to the respective individual canagliflozin dosages but strongly induces sodium excretion in the two higher combination groups (1 mg/kg finerenone and 3 mg/kg canagliflozin; 1 mg/kg finerenone and 10 mg/kg canagliflozin) in comparison to the respective individual finerenone and canagliflozin (SGLT2 inhibitor) dosage groups.

Note that the urinary sodium concentration is statistically significant higher in the combination groups of 3 and 10 mg/kg canagliflozin with 1 mg/kg finerenone, respectively. The urinary sodium excretion of the combination therapy is more than the pure sum of the respective monotherapies. Thereby showing an over-additive effect in comparison to the respective monotherapy. Therefore, the combination of finerenone and canagliflozin (SGLT2 inhibitor) under typical conditions of activated RAAS leads to a significant natriuretic efficacy improvement over the sum of the monotherapies. This natriuretic efficacy improvement is a major clinical goal in the treatment of cardiovascular and/or cardiorenal diseases such as heart failure and CKD.

### B-7 Comparison of Monotherapy (Finerenone or Dapagliflozin (SGLT2 Inhibitor)) with Combination Therapy (Combined Use of Finerenone and Dapagliflozin (SGLT2 Inhibitor)) in Conscious Rats with Activated RAAS

In this comparison, eight different administrations were performed (cf. groups A to H): Group A received the solvent (PEG400) only. This group serves as control group. Groups B, C and D received three increasing dosages of dapagliflozin (SGLT2 inhibitor) only. It serves to detect the effects of the monotherapy with SGLT2 inhibitor. Group E received a finerenone only. It serves to detect the effect of the monotherapy with finerenone. Groups F, G and H received finerenone and the three doses of SGLT2 inhibitor in combination, respectively. It serves to detect the effect of the combined therapy with finerenone and dapagliflozin (SGLT2 inhibitor) (combination according to the invention). The following methods as described in sections B1 (with 10 animals per group) were used.

TABLE 5

| | Urinary Volume/ | Urinary Glucose | Urinary K+ | Urinary Na+ |
|---|---|---|---|---|
| | body weight | concentration | concentration | concentration |
| Group | [ml/kg] | [mmol] | [mmol] | [mmol] |
| A: Solvent | 26.45 ± 2.14 | b.d.l. | 1.91 ± 0.09 | 0.08 ± 0.02 |
| Mean value ± SEM | | | | |
| B: 0.03 mg/kg dapagliflozin | 39.17 ± 4.08 | 0.72 ± 0.18 | 2.21 ± 0.16 | 0.19 ± 0.05 |
| (SGLT2 inhibitor) | | | | |
| (Monotherapy) | | | | |
| Mean value ± SEM | | | | |
| C: 0.3 mg/kg dapagliflozin | 38.84 ± 3.94 | 5.26 ± 0.29 | 2.43 ± 0.07 | 0.19 ± 0.04 |
| (SGLT2 inhibitor) | | | | |
| (Monotherapy) | | | | |
| Mean value ± SEM | | | | |
| D: 3 mg/kg dapagliflozin | 48.72 ± 1.53 | 9.71 ± 0.36 | 2.34 ± 0.10 | 0.29 ± 0.05 |
| (SGLT2 inhibitor) | | | | |
| (Monotherapy) | | | | |
| Mean value ± SEM | | | | |
| E: 1 mg/kg finerenone | 23.44 ± 1.62 | b.d.l. | 1.84 ± 0.10 | 0.33 ± 0.43 |
| (Monotherapy) | | | | |
| Mean value ± SEM | | | | |
| F: Combination group | 36.05 ± 5.04 | 0.74 ± 0.16 | 1.95 ± 0.15 | 0.56 ± 0.07 |
| 0.03 mg/kg dapagliflozin | p < 0.05 vs. E | p < 0.005 vs. E | | p < 0.005 vs. B |
| (SGLT2 inhibitor) + | | | | p < 0.05 vs. E |
| 1 mg/kg finerenone | | | | |
| Mean value ± SEM | | | | |
| G: Combination group | 33.18 ± 1.95 | 4.42 ± 0.36 | 1.74 ± 0.08 | 0.51 ± 0.04 |
| 0.3 mg/kg dapagliflozin | p < 0.01 vs. E | p < 0.005 vs. E | p < 0.005 vs. C | p < 0.005 vs. C |
| (SGLT2 inhibitor) + | | | | p < 0.01 vs. E |
| 1 mg/kg finerenone | | | | |
| Mean value ± SEM | | | | |
| H: Combination group | 50.89 ± 2.00 | 9.84 ± 0.49 | 2.22 ± 0.13 | 0.83 ± 0.12 |
| 3 mg/kg dapagliflozin | p < 0.005 vs. E | p < 0.005 vs. E | p < 0.05 vs. E | p < 0.005 vs. D |
| (SGLT2 inhibitor) + | | | | p < 0.005 vs. E |
| 1 mg/kg finerenone | | | | |
| Mean value ± SEM | | | | |

The results are also depicted in FIG. 5.

FIG. 5: Influence of the SGLT2 inhibitor dapagliflozin (groups B-D), of the MR antagonist finerenone (E), and their combination (groups F-H) on

(a) urinary volume (FIG. 5a),

(b) the urinary glucose concentration (FIG. 5b),

(c) the urinary potassium concentration (FIG. 5c), and

(d) the urinary sodium concentration (FIG. 5d),

of awake Wistar rats with activated renin-angiotensin-aldosterone system, which were examined according to section B above, ‘assessment of the physiological effectiveness’ for 24 hours after oral administration of the substances in metabolic cages. n=10 animals/per group. +: p<0.05 vs. combination group, ++: p<0.01 vs. combination group, +++: p<0.005 vs. combination group, ‘ns’ means not significant vs. combination group. S means solvent (group A). 0.03 mg/kg dapagliflozin (group B), 0.3 mg/kg dapagliflozin (group C), 3 mg/kg dapagliflozin (group D), 1 mg/kg finerenone (group E), 0.03 mg/kg dapagliflozin+1 mg/kg finerenone combination (group F), 0.3 mg/kg dapagliflozin+1 mg/kg finerenone combination (group G), 3 mg/kg dapagliflozin+1 mg/kg finerenone combination (group H).

From table 5 and FIG. 5 a to d, the following can be concluded:

Group A (solvent only), in which the solvent (PEG400) was administered, shows the physiological excretion of volume, potassium and sodium of conscious rats over 24 hours under RAAS activation.

Groups B, C and D (monotherapy dapagliflozin (SGLT2 inhibitor)) show that administration of dapagliflozin has no influence on urinary volume, urinary potassium and urinary sodium at the doses of 0.03 and 0.3 mg/kg while a dose of 3 mg/kg induces an increase in urinary volume and urinary sodium. All doses of dapagliflozin induce a strong increase in urinary glucose.

Group E (monotherapy finerenone) shows that administration of 1 mg/kg of finerenone has no influence on urinary volume, urinary glucose and urinary potassium, but causes an increase in urinary sodium.

Groups F, G and H (combination of finerenone and dapagliflozin (SGLT2 inhibitor)) show that administration of a combination of 1 mg/kg finerenone and 0.03 to 3 mg/kg dapagliflozin does not increase urinary glucose in comparison to the respective individual dapagliflozin dosages (finerenone alone has no effect on urinary glucose), and does not increase urinary volume in comparison to the respective individual dapagliflozin dosages but strongly induces sodium excretion in all combination groups (1 mg/kg finerenone and 0.03 mg/kg dapagliflozin; 1 mg/kg finerenone and 0.3 mg/kg dapagliflozin; 1 mg/kg finerenone and 3 mg/kg dapagliflozin) in comparison to the respective individual finerenone and dapagliflozin (SGLT2 inhibitor) dosage groups.

Note that the urinary sodium concentration is statistically significant higher in all combination groups of 0.03 to 3 mg/kg dapagliflozin with 1 mg/kg finerenone, respectively. The urinary sodium excretion of the combination therapy with 0.03 mg/kg dapagliflozin and 1 mg/kg finerenone and the combination therapy of 3 mg/kg dapagliflozin and 1 mg/kg finerenone is more than the pure sum of the respective monotherapies. Thereby showing an over-additive effect in comparison to the respective monotherapy. Therefore, the combination of finerenone and canagliflozin (SGLT2 inhibitor) under typical conditions of activated RAAS leads to a significant natriuretic efficacy improvement over the sum of the monotherapies. This natriuretic efficacy improvement is a major clinical goal in the treatment of cardiovascular and/or cardiorenal diseases such as heart failure and CKD.

1. A combination comprising finerenone or a hydrate, solvate, pharmaceutically acceptable salt thereof, or a polymorph thereof, and a SGLT2 inhibitor, or a hydrate, solvate, pharmaceutically acceptable salt thereof, or a polymorph thereof.

2. The combination according to claim 1, wherein the SGLT2 inhibitor is selected from the group consisting of canagliflozin, dapagliflozin, empagliflozin, ertugliflozin, ipragliflozin, remogliflozin, sergliflozin and tofogliflozin.

3. The combination according to claim 1, wherein the SGLT2 inhibitor is selected from the group consisting of canagliflozin, dapagliflozin, and empagliflozin.

4. The combination according to claim 1, wherein the combination is selected from the group consisting of or is part of a fixed combination, a single dosage form, two separate dosage forms, a combination pack, a kit-of-parts or a non-fixed combination.

5. The combination according to claim 1, wherein the combination comprises the components:
a. one dosage form comprising finerenone or a hydrate, solvate, pharmaceutically acceptable salt thereof, or a polymorph thereof, and
b. one dosage form comprising a SGLT2 inhibitor a hydrate, solvate, pharmaceutically acceptable salt thereof, or a polymorph thereof.

6. The combination according to claim 5, wherein the components a. and b. are administered separately, sequentially, simultaneously, concurrently or chronologically staggered.

7. The combination according to claim 1, wherein the combination is a single dosage form.

8. The combination according to claim 1, wherein the combination comprises finerenone or a hydrate, solvate, pharmaceutically acceptable salt thereof, or a polymorph thereof, in an amount of 0.25 to 80 mg.

9. The combination according to claim 1, wherein the combination comprises finerenone or a hydrate, solvate, pharmaceutically acceptable salt thereof, or a polymorph thereof and wherein the SGLT2 inhibitor is selected from
empagliflozin or an anhydrate, a hydrate thereof, solvate thereof, pharmaceutically acceptable salt thereof, a prodrug thereof or a polymorph thereof in an amount of 0.5 to 30 mg,
dapagliflozin or an anhydrate, a hydrate thereof, solvate thereof, pharmaceutically acceptable salt thereof, a prodrug thereof or a polymorph thereof in an amount of 0.5 to 20 mg, and
canagliflozin or an anhydrate, a hydrate thereof, solvate thereof, pharmaceutically acceptable salt thereof, a prodrug thereof or a polymorph thereof in an amount of 0.5 mg to 300 mg.

10. The combination according to claim 1 for once daily application.

11. A medicament comprising the combination according to claim 1 and an inert, nontoxic, pharmaceutically suitable excipient.

12. The combination according to claim 11 for use as medicament for treating and/or preventing diseases.

13. The combination according to claim 1 for use as medicament for treating and/or preventing diseases, wherein the diseases is selected from:
cardiovascular disorders such as congestive heart failure, acute heart failure, chronic heart failure, worsening chronic heart failure (WCHF), hospitalization for heart failure, heart failure with preserved ejection fraction (HFpEF), heart failure with mid-range ejection fraction (HFmrEF) or heart failure with reduced ejection fraction (HFrEF);
renal and cardiorenal disorders such as chronic kidney disease (CKD), non-diabetic chronic kidney disease (ndCKD), diabetic kidney disease (DKD), hypertensive kidney disease, cardiorenal syndrome, nephrotic syndrome, hepatorenal syndrome, renal hypoperfusion, intradialytic hypotension, obstructive uropathy, glomerulopathies, IgA nephropathy, glomerulonephritis, glomerulosclerosis, tubulointerstitial diseases, nephropathic diseases such as primary and congenital kidney disease, nephritis, Alport syndrome, kidney inflammation, immunological kidney diseases, kidney transplant rejection, immune complex-induced kidney diseases, nephropathy induced by toxic substances, contrast medium-induced nephropathy; minimal change glomerulonephritis (lipoid), focal segmental glomerulosclerosis (FSGS), amyloidosis, renal cysts, hypertensive nephrosclerosis and nephrotic syndrome (which can be characterized diagnostically, for example, by abnormally reduced creatinine and/or water excretion, abnormally increased blood concentrations of urea, nitrogen, potassium and/or creatinine, altered urine osmolarity or urine volume, increased microalbuminuria, macroalbuminuria, lesions of glomeruli and arterioles, tubular dilatation, hyperphosphataemia and/or the need for dialysis), uraemia, anaemia, electrolyte disturbances (for example hyperkalaemia, hyponatraemia, disturbances in bone and carbohydrate metabolism, polycystic kidney disease (PCKD) and of the syndrome of inadequate ADH secretion (SIADH);
edema, pulmonary edema, cerebral edema, renal edema and heart failure-related edema;
cirrhosis;
NASH (non-alcoholic steatohepatitis);
arterial hypertension, resistant hypertension, pulmonary hypertension, essential hypertension;
cardiovascular disorders such as hypertension, left ventricular dysfunction, hypertrophic cardiomyopathy, diabetic cardiomyopathy, supraventricular arrythmias, ventricular arrythmias, atrial fibrillation, atrial flutter,
cardiovascular disorders such as stable angina pectoris, unstable angina pectoris, myocardial infarction and sequelae thereof, aneurysms, detrimental vascular remodelling, atherosclerosis, atrial fibrillation, stroke;
shock such as cardiogenic shock, septic shock and anaphylactic shock;
hypertensive kidney disease, peripheral arterial disease (PAD) including claudication and including critical limb ischemia, coronary microvascular dysfunction (CMD) including CMD type 1-4, primary and secondary Raynaud's phenomenon, microcirculation disturbances, peripheral and autonomic neuropathies, diabetic microangiopathies, diabetic retinopathy, diabetic limb ulcers, gangrene, CREST syndrome, erythematous disorders, rheumatic diseases, for promoting wound healing, inflammatory diseases, asthmatic diseases, chronic obstructive pulmonary disease (COPD), acute respiratory distress syndrome (ARDS), acute lung injury (ALI), alpha-1-antitrypsin deficiency (AATD), pulmonary fibrosis, pulmonary emphysema (for example smoking-induced pulmonary emphysema) and cystic fibrosis (CF);
lung disorders and cardiopulmonary disorders such as pulmonary hypertension, disorders of the central nervous system;
fibrotic disorders and other disease manifestations (for example end organ damage affecting brain, kidney or heart);
Sleep apnea;
Obesity;
Coronary Artery Disease (CAD);
Acute Kidney Injury (AKI);
Chronic kidney disease after Acute Kidney Injury following Major surgery (AKIM);
multiple insults such as ischemia-reperfusion injury, radiocontrast administration, cardiopulmonary bypass surgery, shock and sepsis.

14. The combination according to claim 13, wherein the diseases is selected from chronic kidney disease (CKD), hypertensive kidney disease, diabetic kidney disease (DKD), non-diabetic chronic kidney disease (ndCKD), chronic kidney disease in patients with type-1-diabetes, chronic kidney disease in patients with type-2-diabetes, diabetic retinopathy, diabetic retinopathy in patients with type-1-diabetes, diabetic retinopathy in patients with type-2-diabetes, worsening chronic heart failure (WCHF), heart failure with preserved ejection fraction (HFpEF), heart failure with mid-range ejection fraction (HFmrEF), heart failure with reduced ejection fraction (HFrEF).

15. A method for preventing and/or treating a cardiovascular and/or renal disease in a subject in need thereof comprising administering to the subject a therapeutically effective amount of the combination according to claim 1.
